(19) United States
(12) Patent Application Publication
(10) Pub. No.: US 2016/0050930 A1
(43) Pub. Date: Feb. 25, 2016

(54) METHODS OF CONTROLLING NEONICOTINOID RESISTANT PESTS

(71) Applicants: SYNGENTA PARTICIPATIONS AG, Basel (CH); SYNGENTA LIMITED, Surrey (GB)

(72) Inventors: Fides Benfatti, Stein (CH); Christopher Richard Ayles Godfrey, Stein (CH); Andre Jeanguenat, Stein (CH); Jagadish Pabba, Goa (IN); Sebastian Rendler, Stein (CH); Jurgen Harry Schaetzer, Stein (CH); Russell Slater, Stein (CH); Andrew Crossthwaite, Berkshire (GB)

(73) Assignees: SYNGENTA LIMITED, Surrey (UK); SYNGENTA PARTICIPATIONS AG, Basel (CH)

(21) Appl. No.: 14/780,575

(22) PCT Filed: Mar. 12, 2014
(86) PCT No.: PCT/EP2014/054849

§ 371 Date: Sep. 28, 2015

(30) Foreign Application Priority Data
Mar. 28, 2013 (IN) 963/DEL/2013

(51) Int. Cl. A01N 43/90; C07D 451/02

(52) U.S. Cl. CPC A01N 43/90; C07D 451/02

(57) ABSTRACT

The present invention relates to a method of controlling insects (in particular insects of the order Hemiptera, especially aphids and whitefly) that are resistant to neonicotinoid insecticides, to methods of controlling insects whereby undesired insects are affected but beneficial arthropods are not affected, using compounds of formula (I) (where A, R1 and R2 are as defined above), and, further, to novel compounds of formula (I) which are useful in the aforementioned methods and/or which possess enhanced insecticidal properties, and to compositions containing said compounds.

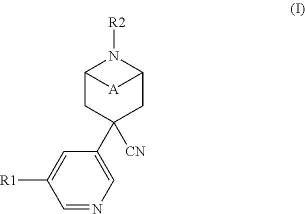

The present invention relates to a method of controlling insects (in particular insects of the order Hemiptera, especially aphids and whitefly) that are resistant to neonicotinoid insecticides, to methods of controlling insects whereby undesired insects are affected but beneficial arthropods are not affected, using compounds of formula I (where A, R1 and R2 are as defined below),

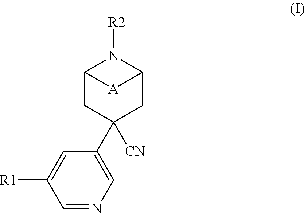

and, further, to novel compounds of formula I which are useful in the aforementioned methods and/or which possess enhanced insecticidal properties, and to compositions containing said compounds.

Bicyclic amine derivatives with insecticidal properties are disclosed, for example, in WO9637494.

There is a continuing need to find new methods of controlling resistant insect populations, as well as more selective methods of controlling insects whereby undesired insects are affected but beneficial arthropods are not affected, and additionally biologically active compounds suitable for use in the aforementioned methods, as well as new biologically active compounds displaying superior properties for use as agrochemical active ingredients (for example, greater biological activity, a different spectrum of activity, an increased safety profile, improved physico-chemical properties, or increased biodegradability).

The damage of plants, and in particular commercial crops, has resulted in large amounts of resources and efforts being spent attempting to control the activities of Hemiptera.

Plants exhibiting aphid damage can possess a variety of symptoms, such as decreased growth rates, mottled leaves, yellowing, stunted growth, curled leaves, browning, wilting, low yields and death. The removal of sap creates a lack of vigour in the plant, and aphid saliva can also be toxic to plants. Many Hemipteran species, transmit disease-causing organisms like plant viruses to their hosts. The green peach aphid (Myzus persicae) is a vector for more than 110 plant viruses. Cotton aphids (Aphis gossypii) are also vectors of several economically important viruses. Whiteflies feed by tapping into the phloem of plants, introducing toxic saliva and decreasing the plants' overall turgor pressure. Since whiteflies congregate in large numbers, susceptible plants can be quickly overwhelmed. Further harm is done by mold growth encouraged by the honeydew that both aphids and whiteflies secrete.

The neonicotinoids represent the fastest-growing class of insecticides introduced to the market since the commercialization of pyrethroids (Nauen & Denholm, 2005: Archives of Insect Biochemistry and Physiology 58:200-215) and are extremely valuable insect control agents not least because they had exhibited little or no cross-resistance to older insecticide classes, which suffer markedly from resistance problems. However, reports of insect resistance to the neonicotinoid class of insecticides are on the increase.

The increase in resistance of such insects to neonicotinoid insecticides thus poses a significant threat to the cultivation of a number of commercially important crops, fruits and vegetables, and there is thus a need to find alternative insecticides capable of controlling neonicotinoid resistant insects (i.e. to find insecticides that do not exhibit any cross-resistance with the neonicotinoid class).

Resistance may be defined as “a heritable change in the sensitivity of a pest population that is reflected in the repeated failure of a product to achieve the expected level of control when used according to the label recommendation for that pest species”. (IRAC)

Cross-resistance occurs when resistance to one insecticide confers resistance to another insecticide via the same biochemical mechanism. This can happen within insecticide chemical groups or between insecticide chemical groups. Cross-resistance may occur even if the resistant insect has never been exposed to one of the chemical classes of insecticide.

Two of the major mechanisms for neonicotinoid resistance include:—
(i) Target site resistance, whereby resistance is associated with replacement of one or more amino acids in the insecticide target protein (i.e. the nicotinic acetylcholine receptor); and
(ii) Metabolic resistance, such as enhanced oxidative detoxification of neonicotinoids due to overexpression of monooxygenases;

The cytochrome P450 monooxygenases are an important metabolic system involved in the detoxification/activation of xenobiotics. As such, P450 monooxygenases play an important role in insecticide resistance. P450 monooxygenases have such a phenomenal array of metabolizable substrates because of the presence of numerous P450s (60-111) in each species, as well as the broad substrate specificity of some P450s. Studies of monooxygenase-mediated resistance have indicated that resistance can be due to increased expression of one P450 (via increased transcription) involved in detoxification of the insecticide and might also be due to a change in the structural gene itself. As such, metabolic cross-resistance mechanisms affect not only insecticides from the given class (e.g. neonicotinoids) but also seemingly unrelated insecticides. For example, cross-resistance relationships between the neonicotinoids and pymetrozine in Bemisia tabaci have been reported by Gorman et al (Pest Management Science 2010, p. 1186-1190).

It has now been surprisingly found that certain bicyclic amines can be successfully used to control neonicotinoid resistant populations of insects in the Hemiptera order.

Thus, in the first aspect of the invention there is provided a method of controlling insects from the order Hemiptera which are resistant to one or more of the neonicotinoid insecticides, which method comprises applying to said neonicotinoid resistant insects a compound of formula (I):

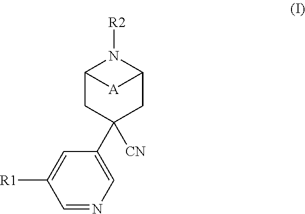

wherein

### A is —CH₂—CH₂— or —CH═CH—;

R¹ is halogen; and
R² is hydrogen, formyl, cyano, hydroxy, NH₂, C₁-C₆alkyl (optionally substituted by aryl, aryloxy, heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl (optionally substituted by aryl or heteroaryl, which themselves can be optionally substituted by one to three substituents independently selected from halogen, cyano, nitro, C₁-C₄alkyl, C₁-C₄haloalkyl, and C₁-C₄alkoxy), C₁-C₄alkoxy(C₁-C₄)alkoxy(C₁-C₄)alkyl, C₁-C₆alkylcarbonyl(C₁-C₆)alkyl, C₁-C₄alkoxyimino(C₁-C₄)alkyl, C₁-C₄haloalkoxy(C₁-C₄)alkyl, C₁-C₆alkoxycarbonyl(C₁-C₆)alkyl, C₁-C₄alkoxy(C₁-C₄)alkoxycarbonyl(C₁-C₆)alkyl, hydroxycarbonyl(C₁-C₆)alkyl, aryloxycarbonyl(C₁-C₆)alkyl (wherein the aryl group can be optionally substituted by one or two substituents independently selected from halogen, cyano, nitro, C₁-C₄alkyl, C₁-C₄ haloalkyl, and C₁-C₄alkoxy), C₁-C₄alkylaminocarbonyl(C₁-C₆)alkyl, di(C₁-C₄alkyl)aminocarbonyl(C₁-C₆)alkyl, C₁-C₄haloalkylaminocarbonyl(C₁-C₆)alkyl, di(C₁-C₄haloalkyl)aminocarbonyl-C₁-C₆alkyl, C₁-C₂alkoxy(C₂-C₄)alkylaminocarbonyl(C₁-C₄)alkyl, C₂-C₆alkenyloxycarbonyl(C₁-C₆)alkyl, C₃-C₆alkynyloxycarbonyl(C₁-C₆)alkyl, (R³O)₂(O═)P(C₁-C₆)alkyl where R³ is hydrogen, C₁-C₄alkyl or benzyl, C₃-C₇cycloalkyl (optionally substituted by one to three substituents independently selected from C₁-C₄alkyl, C₁-C₄haloalkyl, and C₁-C₄alkoxy and, additionally, one of the ring member units can optionally represent C═O or C═NR⁴ where R⁴ is hydrogen, C₁-C₄alkyl, C₁-C₄haloalkyl, C₁-C₄cyanoalkyl, C₁-C₄alkoxy, or C₃-C₆cycloalkyl), C₃-C₇halocycloalkyl, C₃-C₇cycloalkenyl (optionally substituted by one or two substituents independently selected from C₁-C₄alkyl, and C₁-C₄haloalkyl, and, additionally, one of the ring member units can optionally represent C═O), C₃-C₇halocycloalkenyl, C₁-C₆alkyl-S(═O)n¹(C₁-C₆)alkyl where n¹ is 0, 1 or 2 (optionally substituted by aryl or heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₃-C₆alkenyl, C₃-C₆haloalkenyl, aryl(C₃-C₆)alkenyl, C₃-C₆alkynyl, C₃-C₆haloalkynyl, aryl(C₃-C₆)alkynyl, C₃-C₆hydroxyalkynyl, C₁-C₆alkoxycarbonyl (optionally substituted by one to three substituents independently selected from halogen, hydroxy, cyano, C₁-C₄alkoxy, C₁-C₄haloalkyl, and aryl), aryloxycarbonyl (optionally substituted by one to three substituents independently selected from halogen, cyano, nitro, C₁-C₄alkyl, C₁-C₄haloalkyl, C₁-C₄alkoxy), C₃-C₆alkenyloxycarbonyl, C₃-C₆alkynyloxycarbonyl, C₁-C₆alkylcarbonyl, C₁-C₆haloalkylcarbonyl, aminocarbonyl, C₁-C₆alkylaminocarbonyl, di(C₁-C₆)alkylaminocarbonyl, aminothiocarbonyl, C₁-C₆alkylaminothiocarbonyl, di(C₁-C₆)alkylaminothiocarbonyl, C₁-C₆alkoxy, C₃-C₆alkenyloxy, C₃-C₈alkynyloxy, aryloxy (optionally substituted by one to three substituents independently selected from halogen, cyano, nitro, C₁-C₄alkyl, C₁-C₄haloalkyl, and C₁-C₄alkoxy), C₁-C₆alkylamino, di(C₁-C₆)alkylamino, C₃-C₆cycloalkylamino, C₁-C₄alkylthio, C₁-C₄alkylsulfinyl, C₁-C₄alkylsulfonyl, C₁-C₄haloalkylsulfonyl, aryl-S(═O)n² (optionally substituted by one or two substituents independently selected from halogen, nitro, and C₁-C₄alkyl) where n² is 0, 1 or 2, aryl (optionally substituted by one to three substituents independently selected from halogen, cyano, nitro, C₁-C₄alkyl, C₁-C₄haloalkyl, C₁-C₄alkoxy, and C₁-C₄haloalkoxy), heteroaryl (optionally substituted by one to three substituents independently selected from halogen, cyano, nitro, C₁-C₄alkyl, C₁-C₄haloalkyl, C₁-C₄alkoxy, and C₁-C₄haloalkoxy), heterocyclyl (optionally substituted by one to three substituents independently selected from halogen, cyano, nitro, C₁-C₄alkyl, C₁-C₄haloalkyl, C₁-C₄alkoxy, and C₁-C₄haloalkoxy, and, additionally, a ring member unit can optionally represent C═O or C═NR⁵ where R⁵ is hydrogen, C₁-C₄ alkyl, C₁-C₄ haloalkyl, C₁-C₄ cyanoalkyl, C₁-C₄ alkoxy, or C₃-C₆ cycloalkyl), (C₁-C₆alkylthio)carbonyl, (C₁-C₆alkylthio)thiocarbonyl, C₁-C₆alkyl-S(═O)n³(═NR⁶)—C₁-C₄alkyl wherein R⁶ is hydrogen, cyano, nitro, C₁-C₄alkyl and n³ is 0 or 1, or R² represents a group “—C(R⁷)(R⁸)(R⁹)” wherein R⁷ is C₁-C₄alkyl, C₁-C₄haloalkyl, or cyclopropyl, R⁸ is hydrogen, C₁-C₄alkyl, C₁-C₄haloalkyl, or cyclopropyl, preferably hydrogen, R⁹ is cyano, C₁-C₄alkyl, C₂-C₆alkenyl, C₂-C₆haloalkenyl, C₁-C₄alkoxy, C₂-C₅alkynyl, C₂-C₄alkoxycarbonyl, C₁-C₄alkylaminocarbonyl, di(C₁-C₃alkyl)aminocarbonyl, C₁-C₂haloalkylaminocarbonyl, C₃-C₆alkenyloxycarbonyl, C₃-C₄alkynyloxycarbonyl, or C₁-C₃alkylcarbonyl; (arylthio)carbonyl(C₁-C₆)alkyl (wherein aryl can be optionally substituted by one to three substituents independently selected from halogen, cyano, nitro, C₁-C₄alkyl, C₁-C₄haloalkyl, and C₁-C₄alkoxy), (C₁-C₃alkylthio)carbonyl(C₁-C₆)alkyl (optionally substituted by aryl or heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), or an agrochemically acceptable salt, N-oxide or isomer thereof.

Surprisingly, compounds of formula (I) are able to control insects that are resistant to neonicotinoid insecticides whereby resistance is a result of either of the aforementioned mechanisms (target site or metabolic).

Further, it has also been surprisingly found that compounds of formula (I) possess an advantageous safety profile with respect to beneficial arthropods, in particular beneficial insects & predatory mites. More particularly, Orius insidiosus, Orius laevigatus, Orius majusculus, Coccinella septempunctata, Adalia bipunctata, Amblydromalus limonicus, Amblyseius andersoni, Amblyseius barkeri, Amblyseius califomicus, Amblyseius cucumeris, Amblyseius montdorensis, Amblyseius swirskii, Phytoseiulus persimilis, Syrphus spp., and Phytoseiulus persimilis. Most particularly, Orius laevigatus.

Beneficial arthropods form a key component in integrated pest management systems. Such systems have the advantage that they are able to reduce the use of chemical agents, which provides many subsequent environmental and economic benefits & advantages. A variety of arthropods can be present whereby a grower may wish to eliminate one or more arthropod pests using a chemical insecticide whilst minimising the impact on the population of beneficial arthropods in the immediate area. However, the fact that beneficial arthropods share certain biological similarities with agricultural arthropod pests presents a significant challenge. Arthropod pests attack a plant by biting, chewing, sucking, or burrowing into the plant tissue, whereas a beneficial arthropod will most typically only use a plant as a physical support. Nevertheless, beneficial arthropods are exposed to the same environmental conditions (including chemical agents, such as insecticides) as their pest counterparts. One group of arthropods that have more intimate contact with plant materials, and which are of significant benefit to growers, are pollinators (such as honeybees). Accordingly, there is a need for new methods, compounds and compositions for controlling insects whereby undesired insects are affected but beneficial arthropods are not.

Thus, in a second aspect of the invention there is provided a method of controlling insects from the order Hemiptera whereby undesired insects are affected but beneficial arthropods are not affected, which method comprises applying to the insects a compound of formula (I).

In a further aspect of the invention there is provided a method of controlling insects from the order Hemiptera which are resistant to one or more of the neonicotinoid insecticides and whereby undesired insects are affected but beneficial arthropods are not affected, which method comprises applying to said neonicotinoid resistant insects a compound of formula (I).

The compounds of formula (I) can be applied in combination with beneficial arthropods, in particular beneficial insects & predatory mites. This has the advantage that lower rates of the compounds of formula (I) can be applied to effectively control the target pest. Beneficial arthropods are useful in the control of a variety of pest species. Orius bugs in particular feed on inter alia aphids and whiteflies.

Thus, in a yet further aspect of the invention there is provided a method of controlling insects from the order Hemiptera which are resistant to one or more of the neonicotinoid insecticides, which method comprises applying to said neonicotinoid resistant insects a compound of formula (I) and one or more beneficial arthropods.

Preferred beneficial arthropods are beneficial insects & predatory mites. More preferably, Orius insidiosus, Orius laevigatus, Orius majusculus, Coccinella septempunctata, Adalia bipunctata, Amblydromalus limonicus, Amblyseius andersoni, Amblyseius barkeri, Amblyseius califomicus, Amblyseius cucumeris, Amblyseius montdorensis, Amblyseius swirskii, Phytoseiulus persimilis, Syrphus spp., or Phytoseiulus persimilis. The most preferred being Orius laevigatus.

Preferably, the neonicotinoid resistant insects from the Hemiptera order which are controlled by the methods according to the present invention are insects from suborder Sternorrhyncha, especially insects from the Aleyrodidae family and the Aphididae family.

By virtue of the surprising ability of a compound of formula I to control such neonicotinoid resistant insects, the invention also provides a method of protecting a crop of useful plants, wherein said crop is susceptible to and/or under attack from such insects. Such a method involves applying to said crop, treating a plant propagation material of said crop with, and/or applying to said insects, a compound of formula I.

Since the compounds of formula I do not exhibit cross-resistance to neonicotinoid resistant Hemiptera, it may be used in a resistance management strategy with a view to controlling resistance to the neonicotinoid class of insecticides. Such a strategy may involve alternating applications of a compound of formula I and a neonicotinoid insecticide, either on an application by application alternation (including different types of application, such as treatment of plant propagation material and foliar spray), or seasonal/crop alternation basis (e.g. use a compound of formula I on a first crop/for control in a first growing season, and use a neonicotinoid insecticide for a subsequent crop/growing season, or vice versa), and this forms yet a further aspect of the invention.

As mentioned herein, not only are insects from the Hemiptera order pests of a number of commercially important crops, the viruses that these insects carry also pose a threat. With the emergence of resistance to neonicotinoid insecticides, the severity of this threat has increased. Thus, a further aspect of the invention provides a method of controlling a plant virus in a crop of useful plants susceptible to and/or under attack by neonicotinoid resistant insects which carry said plant virus, which method comprises applying to said crop, treating a plant propagation material of said crop with, and/or applying to said insects, a compound of formula I.

Examples of plant viruses that may be controlled according to this aspect of the invention include Sobemovirus, Caulimovirus (Caulimoviridae), Closterovirus (Closteroviridae), Sequivirus (Sequiviridae), Enamovirus (Luteoviridae), Luteovirus (Luteoviridae), Polerovirus (Luteoviridae), Umbravirus, Nanovirus (Nanoviridae), Cytorhabdovirus (Rhabdoviridae), Nucleorhabdovirus (Rhabdoviridae).

These viruses are spread preferably by insects which are one or more of as an example Acyrthosiphum pisum, Aphis citricola, Aphis craccivora, Aphis fabae, Aphis frangulae, Aphis glycines, Aphis gossypii, Aphis nasturtii, Aphis pomi, Aphis spiraecola, Aulacorthum solani, Brachycaudus helichrysi, Brevicoryne brassicae, Diuraphis noxia, Dysaphis devecta, Dysaphis plantaginea, Eriosoma lanigerum, Hyalopterus pruni, Lipaphis erysimi, Macrosiphum avenae, Macrosiphum euphorbiae, Macrosiphum rosae, Myzus cerasi F., Myzus nicotianae, Myzus persicae, Nasonovia ribisnigri, Pemphigus bursarius, Phorodon humuli, Rhopalosiphum insertum Wa, Rhopalosiphum maidis Fitch, Rhopalosiphum padi L., Schizaphis graminum Rond., Sitobion avenae, Toxoptera aurantii, Toxoptera citricola, Phylloxera vitifoliae, Bemisia tabaci, Myzus persicae, Nilaparvata lugens, Aphis gossypii, Trialeurodes vaporariorum, Bactericera cockerelli.

Methods of the invention as described herein may also involve a step of assessing whether insects are resistant to neonicotinoid insecticides and/or whether said insects carry a plant virus. This step will in general involve collecting a sample of insects from the area (e.g. crop, field, habitat) to be treated, before actually applying a compound of formula I, and testing (for example using any suitable phenotypic, biochemical or molecular biological technique applicable) for resistance/sensitivity and/or the presence or absence of a virus.

The term neonicotinoid insecticide as used herein refers to any insecticidal compound that acts at the insect nicotinic acetylcholine receptor, and in particular refers to those compounds classified as neonicotinoid insectides according to Yamamoto (1996, Agrochem Jpn 68:14-15). Examples of neonicotinoid insecticides include those in Group 4A and 4C of the IRAC (insecticide resistance action committee, Crop Life) mode of action classification scheme, e.g. acetamiprid, clothianidin, dinotefuran, imidacloprid, nitenpyram, thiacloprid, sulfoxaflor and thiamethoxam, as well as any compound having the same mode of action.

By the terms “control” or “controlling” as applied to insects, it is meant that the targeted insects are repelled from or less attracted to the crops to be protected. Additionally, as applied to insects, the terms “control” or “controlling” may also refer to the inability, or reduced ability, of the insects to feed or lay eggs. These terms may further include that the targeted insects are killed.

Thus the method of the invention may involve the use of an amount of the active ingredient that is sufficient to repel insects (i.e a repellently effective amount of active ingredient), an amount of the active ingredient that is sufficient to stop insects feeding, or it may involve the use of an insecticidally effective amount of active ingredient (i.e. an amount sufficient to kill insects), or any combination of the above effects. Where the terms “control” or “controlling” are applied to viruses it is meant that the level of viral infection of a crop of useful plants is lower than would be observed in the absence of any application of a compound of formula I.

The terms “applying” and “application” are understood to mean direct application to the insect to be controlled, as well as indirect application to said insect, for example through application to the crop or plant on which the insect acts as pest, or to the locus of said crop or insect, or indeed through treatment of the plant propagation material of said crop of plant.

Thus a compound of formula I may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the plant propagation material, such as seed, before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

Pesticidal agents or compound referred to herein using their common name are known, for example, from “The Pesticide Manual”, 15th Ed., British Crop Protection Council 2009.

The term “beneficial” arthropod or insect as used herein refers to any arthropod or insect which has at least one life stage which has a negative impact on arthropod or insect agricultural pests and/or which pollinate crop plants. The term specifically includes arthropods classed as so-called parasitoids due to their tendency to lay eggs on or in an arthropod host. Thus beneficials include pollinators, parasitoids and predators, examples include but are not limited to: Cryptolaemus montrouzieri, Encarsia formosa, Eretmocerus eremicus, Eretmocerus mundus, Feltiella acarisuga Macrophus pygmeus, Nesidiocoris tenuis, aphid midge, centipedes, ground beetles such as Pterostichus melanarius, Agonum dorsale, and Nebria brevicollis, lady beetles such as Adalia bipunctata and Coccinella septempunctata, lacewings such as Chrysoperia carnea, hoverflies such as Syrphus spp., Phytoseiulus persimilis, pirate bugs such as Orius insidiosus, Orius laevigatus, Orius majusculus, predatory mites such as Amblydromalus limonicus, Amblyseius andersoni, Amblyseius barkeri, Amblyseius californicus, Amblyseius cucumeris, Amblyseius montdorensis, Amblyseius swirskii, Phytoseiulus persimilis, predatory midges such as Aphidoletes aphidimyza, rove beetle, tachnid flies, and wasps such as Dacnusa sibirica, Diglyphus isaea Trichogramma brassicae as well as ichneumonid wasps, chalcid wasps and braconid wasps such as Aphidius colemani, Aphidius ervi, Aphidius matrcariae.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The methods of the invention are particularly applicable to the control of neonicotinoid resistant insects (and neonicotinoid resistance in insects) of the order Hemiptera, such as: Acyrthosiphum pisum, Aphis citricola, Aphis craccivora, Aphis fabae, Aphis frangulae, Aphis glycines, Aphis gossypii, Aphis nasturtii, Aphis pomi, Aphis spiraecola, Aulacorthum solani, Brachycaudus helichrysi, Brevicoryne brassicae, Diuraphis noxia, Dysaphis devecta, Dysaphis plantaginea, Eriosoma lanigerum, Hyalopterus pruni, Lipaphis erysimi, Macrosiphum avenae, Macrosiphum euphorbiae, Macrosiphum rosae, Myzus cerasi F., Myzus nicotianae, Myzus persicae, Nasonovia ribisnigri, Pemphigus bursarius, Phorodon humuli, Rhopalosiphum insertum Wa, Rhopalosiphum maidis Fitch, Rhopalosiphum padi L., Schizaphis graminum Rond., Sitobion avenae, Toxoptera aurantii, Toxoptera citricola, Phylloxera vitifoliae, Acyrthosiphon dirhodum, Acyrthosiphon solani, Aphis forbesi, Aphis grossulariae, Aphis idaei, Aphis illinoisensis, Aphis maidiradicis, Aphis ruborum, Aphis schneideri, Brachycaudus persicaecola, Cavariella aegopodii Scop., Cryptomyzus galeopsidis, Cryptomyzus ribis, Hyadaphis pseudobrassicae, Hyalopterus amygdali, Hyperomyzus pallidus, Macrosiphoniella sanborni, Metopolophium dirhodum, Myzus malisuctus, Myzus varians, Neotoxoptera sp, Nippolachnus piri Mats., Oregma lanigera Zehnter, Rhopalosiphum fitchii Sand., Rhopalosiphum nymphaeae, Rhopalosiphum sacchari Ze, Sappaphis piricola Okam.+T, Schizaphis piricola, Toxoptera theobromae Sch, and Phylloxera coccinea, Aleurodicus dispersus, Aleurocanthus spiniferus, Aleurocanthus woglumi, Aleurodicus cocois, Aleurodicus destructor, Aleurolobus barodensis, Aleurothrixus floccosus, Bemisia tabaci, Bemisia argentifolli, Dialeurodes citri, Dialeurodes citrifolli, Parabemisia myricae, Trialeurodes packardi, Trialeurodes ricini, Trialeurodes vaporariorum, Trialeurodes variabilis, Agonoscena targionii, Bactericera cockerelli, Cacopsylla pyri, Cacopsylla pyricola, Cacopsylla pyrisuga, Diaphorina citri, Glycaspis brimblecombei, Paratrioza cockerelli, Troza erytreae, Amarasca biguttula biguttula, Amritodus atkinsoni, Cicadella viridis, Cicadulina mbila, Cofana spectra, Dalbulus maidis, Empoasca decedens, Empoasca biguttula, Empoasca fabae, Empoasca vitis, Empoasca papaya, Idioscopus clypealis, Jacobiasca lybica, Laodelphax striatellus, Myndus crudus, Nephotettix virescens, Nephotettix cincticeps, Nilaparvata lugens, Peregrinus maidis, Perkinsiella saccharicida, Perkinsiella vastatrix, Recilia dorsalis, Sogatella furcifera, Tarophagus Proserpina, Zygina flammigera, Acanthocoris scabrator, Adelphocoris lineolatus, Amblypelta nitida, Bathycoelia thalassina, Blissus leucopterus, Clavigralla tomentosicollis, Edessa meditabunda, Eurydema pulchrum, Eurydema rugosum, Eurygaster Maura, Euschistus servus, Euschistus tristigmus, Euschistus heros Helopeltis antonii, Horcias nobilellus, Leptocorisa acuta, Lygus lineolaris, Lygus hesperus, Murgantia histrionic, Nesidiocoris tenuis, Nezara viridula, Oebalus insularis, Scotinophara coarctata,

Specific examples of neonicotinoid resistant Hemiptera include Bemisia tabaci, Myzus persicae, Nilaparvata lugens, Aphis gossypii, Trialeurodes vaporariorum, Bactericera cockerelli.

Preferably, the neonicotinoid resistant insects are one or more of as an example Acyrthosiphum pisum, Aphis citricola, Aphis craccivora, Aphis fabae, Aphis frangulae, Aphis glycines, Aphis gossypii, Aphis nasturtii, Aphis pomi, Aphis spiraecola, Aulacorthum solani, Brachycaudus helichrysi, Brevicoryne brassicae, Diuraphis noxia, Dysaphis devecta, Dysaphis plantaginea, Eriosoma lanigerum, Hyalopterus pruni, Lipaphis erysimi, Macrosiphum avenae, Macrosiphum euphorbiae, Macrosiphum rosae, Myzus cerasi F., Myzus nicotianae, Myzus persicae, Nasonovia ribisnigri, Pemphigus bursarius, Phorodon humuli, Rhopalosiphum insertum Wa, Rhopalosiphum maidis Fitch, Rhopalosiphum padi L., Schizaphis graminum Rond., Sitobion avenae, Toxoptera aurantii, Toxoptera citricola, Phylloxera vitifoliae, Bemisia tabaci, Myzus persicae, Nilaparvata lugens, Aphis gossypii, Trialeurodes vaporariorum, Bactericera cockerelli.

More preferably, the neonicotinoid resistant insects are one or more of as an example Bemisia tabaci, Myzus persicae, Nilaparvata lugens, Aphis gossypii, Trialeurodes vaporariorum, Bactericera cockerelli.

Most preferably the neonicotinoid resistant insects are Bemisia tabaci or Myzus persicae.

Since the methods of the invention have the effect of controlling insect pest and or viral infestation in crops of useful plants, said methods may also be viewed as methods of improving and/or maintaining plant health in said crops or as methods of increasing/maintaining the well-being of a crop.

Crops of useful plants in which the composition according to the invention can be used include perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp and jute; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria, especially those of the genus Bacillus. Further examples of toxins which can be expressed include δ-endotoxins, vegetative insecticidal proteins (Vip), insecticidal proteins of bacteria colonising nematodes, and toxins produced by scorpions, arachnids, wasps and fungi.

Example crops include: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Crops are to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The term “plant” or “useful plants” as used herein includes seedlings, bushes and trees. The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, from Bacillus cereus or Bacillus popilliae; or insecticidal proteins from Bacillus thuringiensis, such as δ-endotoxins, e.g. CryIAb, CryIAc, CryIF, CryIFa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated CryIAb, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a CryIAb toxin); YieldGard Rootworm® (maize variety that expresses a Cry3Bb1 toxin); YieldGard Plus® (maize variety that expresses a CryIAb and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a CryIAc toxin); Bollgard I® (cotton variety that expresses a CryIAc toxin); Bollgard II® (cotton variety that expresses a CryIAc and a Cry2Ab toxin); VipCot® (cotton variety that expresses a Vip3A and a CryIAb toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated CryIAb toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a CryIAb toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St.

Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CryIAb toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®.

Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavour).

The table below lists key aphids (as an example of a family of Hemiptera) and crops they target.

| PEST | COMMON NAME | EXAMPLES OF CROPS |
|---|---|---|
| Acyrthosiphum pisum | Pea aphid | pea |
| Aphis citricola | Citrus aphid | citrus |
| Aphis craccivora | Cowpea aphid | vegetables, beans, |
| | | sugarbeet |
| Aphis fabae | Black bean aphid | vegetables, beans, |
| | | sugarbeet |
| Aphis frangulae | Breaking buckthorn | cotton potato |
| | aphid | |
| Aphis glycines | Soybean aphid | soybean |
| Aphis gossypii | Cotton aphid | cotton, vegetables, |
| | | citrus, potato |
| Aphis nasturtii | Buckthorn aphid | potato |
| Aphis pomi | Green apple aphid | apple |
| Aphis spiraecola | Green citurs aphis | apple, citrus, papaya |
| Aulacorthum solani | Foxglove aphid | citrus, sugar beet |
| Brachycaudus | Plum aphid | peach, stone fruits |
| helichrysi | | |
| Brevicoryne brassicae | Cabbage aphid | brassica |
| Diuraphis noxia | Russion wheat aphid | cereals |
| Dysaphis devecta | Leaf-curling aphid | pome fruits |
| Dysaphis plantaginea | Rosy apple aphid | pome fruits, stone fruits |
| Eriosoma lanigerum | Wooly apple aphid | pome fruits, stone fruits |
| Hyalopterus pruni | Mealy plum aphid | stone fruits |
| Lipaphis erysimi | False cabbage aphid | brassica |
| Macrosiphum avenae | Grain aphid | cereals |
| Macrosiphum | Potato aphid | potato, sugar beet, |
| euphorbiae | | vegetables |
| Macrosiphum rosae | Rose aphid | ornamentals |
| Myzus cerasi F. | Black cherry aphid | cherry, stone fruits |
| Myzus nicotianae | Tobacco aphid | tobacco |
| Myzus persicae | Peach aphid | peach, deciduous fruits, |
| | | vegetables, sugarbeet, |
| | | potato, cereals, |
| | | sugarcane, maize, |
| | | ornamentals |
| Myzus persicae | Green peach aphid | peach, deciduous fruits, |
| | | vegetables, sugarbeet, |
| | | potato, cereals, |
| | sugarcane, maize, | |
| | ornamentals | |
| Nasonovia ribisnigri | Lettuce aphid | vegetables |
| Pemphigus bursarius | Lettuce root aphid | vegetables |
| Phorodon humuli | Hop aphid | hops |
| Rhopalosiphum | Apple-grass aphid | Deciduous fruits, |
| insertum Wa | | ornamentals |
| Rhopalosiphum maidis | Corn leaf aphid | Maize, cereals |
| Fitch | | |
| Rhopalosiphum padi L. | Wheat aphid | Maize, cereals |
| Schizaphis graminum | Spring grain aphid | cereals |
| Rond. | | |
| Sitobion avenae | Wheat aphid | cereals |
| Toxoptera aurantii | Citrus aphid | citrus |
| Toxoptera citricola | Black citrus aphid | citrus |
| Phylloxera vitifoliae | Grape Phylloxera | vine |

The table below lists key whitefly and crops they target.

| PEST | COMMON NAME | EXAMPLES OF CROPS |
|---|---|---|
| Aleurocanthus | Orange spiney | Citrus |
| spiniferus | whitefly | |
| Aleurocanthus | Citrus blackfly | Citrus, Coffee |
| woglumi | | |
| Aleurodicus | Coconut whitefly | Coconut, Cashew |
| cocois | | |
| Aleurodicus | Coconut whitefly | Coconut, Pepper |
| destructor | | |
| Aleurodicus | Spiralling whitefly | Citrus, Coconut, Soybean, |
| disperses | | Cassava, Stone Fruit, Coffee, |
| | | vegetables |
| Aleurothrixus | Wooly whitefly | Citrus, Mango, Coffee |
| floccosus | | |
| Bemisia tabaci | Tobacco whitefly | Vegetables, Cotton, Crucifera, |
| | Silverleaf whitefly | Legunes, Soyabean, Tobacco, |
| | | Potato. |
| Dialeurodes citri | Citrus whitelfy | Citrus |
| Parabemisia | Bayberry whitefly | Citrus, vegetables |
| myricae | | |
| Trialeurodes | Glasshouse | Melon, vegetables, Legumes, |
| vaporariorum | whitefly | Roses |

The table below lists key planthoppers and crops they target.

| PEST | COMMON NAME | EXAMPLES OF CROPS |
|---|---|---|
| Laodelphax | Small brown | Rice |
| striatellus | planthopper | |
| Nilaparvata lugens | Brown | Rice |
| | planthopper | |
| Sogatella furcifera | White backed | Rice |
| | planthopper | |

Accordingly, as used herein, part of a plant includes propagation material. There may be mentioned, e.g., the seeds (in the strict sense), roots, fruits, tubers, bulbs, rhizomes, parts of plants. Germinated plants and young plants, which are to be transplanted after germination or after emergence from the soil, may also be mentioned. These young plants may be protected before transplantation by a total or partial treatment by immersion.

Parts of plant and plant organs that grow at later point in time are any sections of a plant that develop from a plant propagation material, such as a seed. Parts of plant, plant organs, and plants can also benefit from the pest damage protection achieved by the application of the compound on to the plant propagation material. In an embodiment, certain parts of a plant and certain plant organs that grow at later point in time can also be considered as plant propagation material, which can themselves be applied (or treated) with the compound; and consequently, the plant, further parts of the plant and further plant organs that develop from the treated parts of plant and treated plant organs can also benefit from the pest damage protection achieved by the application of the compound on to the certain parts of plant and certain plant organs.

Methods for applying or treating pesticidal active ingredients on to plant propagation material, especially seeds, are known in the art, and include dressing, coating, pelleting and soaking application methods of the propagation material. It is preferred that the plant propagation material is a seed.

Although it is believed that the present method can be applied to a seed in any physiological state, it is preferred that the seed be in a sufficiently durable state that it incurs no damage during the treatment process. Typically, the seed would be a seed that had been harvested from the field; removed from the plant; and separated from any cob, stalk, outer husk, and surrounding pulp or other non-seed plant material. The seed would preferably also be biologically stable to the extent that the treatment would cause no biological damage to the seed. It is believed that the treatment can be applied to the seed at any time between harvest of the seed and sowing of the seed or during the sowing process (seed directed applications). The seed may also be primed either before or after the treatment.

Even distribution of the compound and adherence thereof to the seeds is desired during propagation material treatment. Treatment could vary from a thin film (dressing) of a formulation containing the compound, for example, a mixture of active ingredient(s), on a plant propagation material, such as a seed, where the original size and/or shape are recognizable to an intermediary state (such as a coating) and then to a thicker film (such as pelleting with many layers of different materials (such as carriers, for example, clays; different formulations, such as of other active ingredients; polymers; and colourants) where the original shape and/or size of the seed is no longer recognisable into the controlled release material or applied between layers of materials, or both.

The seed treatment occurs to an unsown seed, and the term “unsown seed” is meant to include seed at any period between the harvest of the seed and the sowing of the seed in the ground for the purpose of germination and growth of the plant.

Treatment to an unsown seed is not meant to include those practices in which the active ingredient is applied to the soil but would include any application practice that would target the seed during the planting process.

Preferably, the treatment occurs before sowing of the seed so that the sown seed has been pre-treated with the compound. In particular, seed coating or seed pelleting are preferred in the treatment of the compound. As a result of the treatment, the compound is adhered on to the seed and therefore available for pest control.

The treated seeds can be stored, handled, sowed and tilled in the same manner as any other active ingredient treated seed.

The compounds of formula (I) may exist in different geometric or optical isomers or tautomeric forms. This invention covers all such isomers and tautomers and mixtures thereof in all proportions as well as isotopic forms such as deuterated compounds. The invention also covers salts and N-oxides.

The compounds of the invention may contain one or more asymmetric carbon atoms, and may exist as enantiomers (or as pairs of diastereoisomers) or as mixtures of such. It is, however, preferred that a cis relative stereochemical configuration exists between the “CN” group and the “A” group of the central core structure.

Where a group has more than one substituent the substituents may be the same or different.

Alkyl groups (either alone or as part of a larger group, such as alkoxy-, alkylthio-, alkylsulfinyl-, alkylsulfonyl-, alkylcarbonyl- or alkoxycarbonyl-) can be in the form of a straight or branched chain and are, for example, methyl, ethyl, propyl, prop-2-yl, butyl, but-2-yl, 2-methyl-prop-1-yl or 2-methyl-prop-2-yl. The alkyl groups are preferably C₁-C₆, more preferably C₁-C₄, most preferably C₁-C₃ alkyl groups. Where an alkyl moiety is said to be substituted, the alkyl moiety is preferably substituted by one to four substituents, most preferably by one to three substituents.

Alkylene groups can be in the form of a straight or branched chain and are, for example, —CH₂—, —CH₂—CH₂—, —CH(CH₃)—, —CH₂—CH₂—CH₂—, —CH(CH₃)—CH₂—, or —CH(CH₂CH₃)—. The alkylene groups are preferably C₁-C₃, more preferably C₁-C₂, most preferably C₁ alkylene groups.

Alkenyl groups can be in the form of straight or branched chains, and can be, where appropriate, of either the (E)- or (Z)-configuration. Examples are vinyl and allyl. The alkenyl groups are preferably C₂-C₆, more preferably C₂-C₄, most preferably C₂-C₃ alkenyl groups.

Alkynyl groups can be in the form of straight or branched chains. Examples are ethynyl and propargyl. The alkynyl groups are preferably C₂-C₆, more preferably C₂-C₅, most preferably C₂-C₄ alkynyl groups.

Halogen is fluorine, chlorine, bromine or iodine.

Haloalkyl groups (either alone or as part of a larger group, such as haloalkoxy-, haloalkylthio-, haloalkylsulfinyl- or haloalkylsulfonyl-) are alkyl groups which are substituted by one or more of the same or different halogen atoms and are, for example, difluoromethyl, trifluoromethyl, chlorodifluoromethyl or 2,2,2-trifluoro-ethyl.

Haloalkenyl groups are alkenyl groups which are substituted by one or more of the same or different halogen atoms and are, for example, 2,2-difluoro-vinyl or 1,2-dichloro-2-fluoro-vinyl.

Haloalkynyl groups are alkynyl groups which are substituted by one or more of the same or different halogen atoms and are, for example, 1-chloro-prop-2-ynyl.

Cycloalkyl groups or carbocyclic rings can be in mono- or bi-cyclic form and are, for example, cyclopropyl, cyclobutyl, cyclohexyl and bicyclo[2.2.1]heptan-2-yl. The cycloalkyl groups are preferably C₃-C₈, more preferably C₃-C₆ cycloalkyl groups. Where a cycloalkyl moiety is said to be substituted, the cycloalkyl moiety is preferably substituted by one to four substituents, most preferably by one to three substituents.

Aryl groups (either alone or as part of a larger group, such as aryloxy) are aromatic ring systems which can be in mono-, bi- or tricyclic form. Examples of such rings include phenyl, naphthyl, anthracenyl, indenyl or phenanthrenyl. Preferred aryl groups are phenyl and naphthyl, phenyl being most preferred. Where an aryl moiety is said to be substituted, the aryl moiety is preferably substituted by one to four substituents, most preferably by one to three substituents.

Heteroaryl groups (either alone or as part of a larger group, such as heteroaryl-alkylene-) are aromatic ring systems containing at least one heteroatom and consisting either of a single ring or of two or more fused rings. Preferably, single rings will contain up to three heteroatoms and bicyclic systems up to four heteroatoms which will preferably be chosen from nitrogen, oxygen and sulfur. Examples of monocyclic groups include pyridyl, pyridazinyl, pyrimidinyl, pyrazinyl, pyrrolyl, pyrazolyl, imidazolyl, triazolyl (e.g. [1,2,4]triazolyl), furanyl, thiophenyl, oxazolyl, isoxazolyl, oxadiazolyl, thiazolyl, isothiazolyl and thiadiazolyl. Examples of bicyclic groups include purinyl, quinolinyl, cinnolinyl, quinoxalinyl, indolyl, indazolyl, benzimidazolyl, benzothiophenyl and benzothiazolyl. Monocyclic heteroaryl groups are preferred, pyridyl being most preferred. Where a heteroaryl moiety is said to be substituted, the heteroaryl moiety is preferably substituted by one to four substituents, most preferably by one to three substituents.

Heterocyclyl groups or heterocyclic rings (either alone or as part of a larger group, such as heterocyclyl-alkyl) are non-aromatic ring structures containing up to 10 atoms including one or more (preferably one, two or three) heteroatoms selected from O, S and N. Examples of monocyclic groups include, oxetanyl, 4,5-dihydro-isoxazolyl, thietanyl, pyrrolidinyl, tetrahydrofuranyl, [1,3]dioxolanyl, piperidinyl, piperazinyl, [1,4]dioxanyl, imidazolidinyl, [1,3,5]oxadiazinanyl, hexahydro-pyrimidinyl, [1,3,5]triazinanyl and morpholinyl or their oxidised versions such as 1-oxo-thietanyl and 1,1-dioxo-thietanyl. Examples of bicyclic groups include 2,3-dihydro-benzofuranyl, benzo[1,4]dioxolanyl, benzo[1,3]dioxolanyl, chromenyl, and 2,3-dihydro-benzo[1,4]dioxinyl. Where a heterocyclyl moiety is said to be substituted, the heterocyclyl moiety is preferably substituted by one to four substituents, most preferably by one to three substituents.

Preferred values of A, R¹ and R² are, in any combination, as set out below.

Preferably A is —CH═CH—.

Preferably R¹ is F, Cl or Br; more preferably R¹ is Cl or Br; most preferably R¹ is Br. Alternatively, it is preferred that R¹ is F, I, or Br; more preferred that R¹ is F, or Br; and most preferred that R¹ is F.

Preferably R² is hydrogen, formyl, C₁-C₆alkyl (optionally substituted by phenyl, heteroaryl (wherein heteroaryl is pyridyl, thiophenyl, oxazolyl, isoxazolyl, oxadiazolyl, thiazolyl, isothiazolyl or thiadiazolyl) or heterocyclyl (wherein heterocyclyl is tetrahydrofuranyl, [1,3]dioxolanyl, oxetanyl, thietanyl, 1-oxo-thietanyl or 1,1-dioxo-thietanyl), which themselves can be optionally substituted by one or two substituents independently selected from halogen, cyano, C₁-C₄alkyl, C₁-C₄haloalkyl, and C₁-C₄alkoxy), C₁-C₆haloalkyl (optionally substituted by one or two substituents independently selected from C₁-C₄-alkoxy, tri(C₁-C₄alkyl)silyloxy), and C₃-C₅alkenyl), C₁-C₆cyanoalkyl, C₁-C₆alkoxy(C₁-C₆)alkyl, C₁-C₆alkylcarbonyl(C₁-C₆)alkyl, C₁-C₆alkoxycarbonyl(C₁-C₆)alkyl, C₁-C₄alkylaminocarbonyl(C₁-C₆)alkyl, di(C₁-C₄alkyl)aminocarbonyl(C₁-C₆)alkyl, C₁-C₄haloalkylaminocarbonyl(C₁-C₆)alkyl, C₂-C₆alkenyloxycarbonyl(C₁-C₆)alkyl, C₃-C₆alkynyloxycarbonyl(C₁-C₃)alkyl, C₃-C₆cycloalkyl (optionally substituted by one or two substituents independently selected from C₁-C₄alkyl, and C₁-C₄haloalkyl, and, additionally, one of the ring member units can optionally represent C═O), C₃-C₆halocycloalkyl, C₄-C₇cycloalkenyl (optionally substituted by one or two substituents independently selected from C₁-C₄alkyl, and C₁-C₄haloalkyl, and, additionally, one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, phenyl(C₃-C₆)alkenyl, C₃-C₆alkynyl, C₃-C₆haloalkynyl, C₁-C₆alkoxy, heterocyclyl (wherein heterocyclyl is oxetanyl, 3-tetrahydrofuranyl-2-one, 1,1-dioxo-thietanyl, 1-oxo-thietanyl or thietanyl, and is optionally substituted by one or two substituents independently selected from halogen, cyano, C₁-C₄alkyl, C₁-C₄haloalkyl, and C₁-C₄alkoxy, and, additionally, a ring member unit can optionally represent C═O or C═NR⁵ where R⁵ is C₁-C₄ alkyl, or C₁-C₄ alkoxy); or R² represents the group “—C(R⁷)(R⁹)(R⁹)” wherein R⁷ is C₁-C₄alkyl, C₁-C₄haloalkyl, or cyclopropyl; R⁹ is hydrogen, C₁-C₄alkyl, C₁-C₄haloalkyl, or cyclopropyl, preferably hydrogen; and R⁹ is cyano, C₁-C₄alkyl, C₂-C₆alkenyl, C₂-C₆haloalkenyl, C₁-C₄alkoxy, C₂-C₅alkynyl, C₂-C₄alkoxycarbonyl, C₁-C₄alkylaminocarbonyl, di(C₁-C₃alkyl)aminocarbonyl, C₁-C₂haloalkylaminocarbonyl, C₃-C₆alkenyloxycarbonyl, C₃-C₄alkynyloxycarbonyl, or C₁-C₃alkylcarbonyl.

More preferably R² is hydrogen, formyl, C₁-C₆alkyl (optionally substituted by heteroaryl (wherein heteroaryl is pyridyl, oxazolyl or oxadiazolyl) or heterocyclyl (wherein heterocyclyl is [1,3]dioxolanyl, thietanyl, 1-oxo-thietanyl, 1,1-dioxo-thietanyl or tetrahydrofuranyl), which themselves can be optionally substituted by one or two substituents independently selected from halogen, and C₁-C₄alkyl), C₁-C₆haloalkyl, C₁-C₃cyanoalkyl, C₁-C₄alkoxy(C₁-C₂)alkyl, C₁-C₃alkylcarbonyl(C₁-C₂)alkyl, C₁-C₄alkoxycarbonyl(C₁-C₂)alkyl, C₁-C₄alkylaminocarbonyl(C₁-C₂)alkyl, di(C₁-C₃alkyl)aminocarbonyl(C₁-C₂)alkyl, C₁-C₂haloalkylaminocarbonyl(C₁-C₂)alkyl, C₃-C₆alkenyloxycarbonyl(C₁-C₂)alkyl, C₃-C₄alkynyloxycarbonyl(C₁-C₂)alkyl, C₃-C₆cycloalkyl (optionally substituted by one or two C₁-C₄alkyl substituents and, additionally, one of the ring member units can optionally represent C═O), C₃-C₆halocycloalkyl, C₅-C₆cycloalkenyl (optionally substituted by one or two C₁-C₂alkyl substituents, and, additionally, one of the ring member units can optionally represent C═O), C₁-C₂alkyl-S(═O)n¹(C₁-C₄)alkyl where n¹ is 0, 1 or 2, C₃-C₅alkenyl, C₃-C₅haloalkenyl, C₃-C₆alkynyl; heterocyclyl (wherein heterocyclyl is 1,1-dioxo-thietanyl, 1-oxo-thietanyl or thietanyl) or R² represents the group “—C(R⁷)(R⁹)(R⁹)” wherein R⁷ is C₁-C₃alkyl; R⁹ is hydrogen, or C₁-C₃alkyl, preferably hydrogen; and R⁹ is cyano, C₂-C₄alkenyl, C₂-C₄haloalkenyl, C₂-C₄alkynyl, C₂-C₄alkoxycarbonyl, or C₃-C₆alkenyloxycarbonyl.

Even more preferably R² is hydrogen, formyl, C₁-C₄alkyl (optionally substituted by heterocyclyl, wherein heterocyclyl is [1,3]dioxolanyl, or tetrahydrofuranyl), C₁-C₆haloalkyl, C₁-C₃cyanoalkyl, C₁-C₂alkoxy(C₁-C₂)alkyl, C₁-C₃alkylcarbonyl(C₁-C₂)alkyl, C₁-C₄alkoxycarbonyl(C₁-C₂)alkyl, C₃-C₅alkenyloxycarbonyl(C₁-C₂)alkyl, C₃-C₄alkynyloxycarbonyl(C₁-C₂)alkyl, C₃-C₆cycloalkyl, C₁-C₂alkyl-S(═O)n¹(C₁-C₄)alkyl where n¹ is 0, C₃-C₅alkenyl, C₃-C₅haloalkenyl, C₃-C₆alkynyl; or R² represents the group “—C(R⁷)(R⁹)(R⁹)” wherein R⁷ is C₁-C₂alkyl; R⁹ is hydrogen or C₁-C₂alkyl, preferably hydrogen; and R⁹ is cyano, C₂alkenyl, C₂alkynyl, C₂-C₄alkoxycarbonyl, or C₃-C₅alkenyloxycarbonyl.

Yet more preferably R² is hydrogen, formyl, C₁-C₄alkyl, C₁-C₆haloalkyl, C₁-C₂alkoxy(C₁-C₂)alkyl, C₁-C₄alkoxycarbonyl(C₁-C₂)alkyl, C₃cycloalkyl, C₁-C₂alkyl-S(═O)n¹(C₁-C₄)alkyl where n¹ is 0, C₃-C₅alkenyl, C₃-C₄haloalkenyl, C₃-C₆alkynyl; or R² represents the group “—C(R⁷)(R⁹)(R⁹)” wherein R⁷ is C₁-C₂alkyl; R⁹ is hydrogen or C₁-C₂alkyl, preferably hydrogen; and R⁹ is cyano, C₂alkenyl, C₂alkynyl, C₂-C₄alkoxycarbonyl, or C₃-C₅alkenyloxycarbonyl.

More preferably still R² is C₂-C₄alkyl, C₂-C₄haloalkyl, C₁-C₄alkoxycarbonyl(C₁-C₂)alkyl, C₁-C₂alkoxy(C₁-C₂)alkyl, C₁-C₂alkyl-S(═O)n¹(C₁-C₂)alkyl where n¹ is 0, C₃haloalkenyl, or C₃-C₆alkynyl.

Most preferably R² is C₃-C₄alkynyl.

Embodiments according to the invention are provided as set out below.

Embodiment 1 provides a method of controlling insects from the order Hemiptera which are resistant to one or more of the neonicotinoid insecticides, which method comprises applying to said neonicotinoid resistant insects a compound of formula (I), or an agrochemically acceptable salt, N-oxide or isomer thereof, as defined above.

Embodiment 2 provides a method according to embodiment 1 wherein R² is hydrogen, formyl, C₁-C₆alkyl (optionally substituted by phenyl, heteroaryl (wherein heteroaryl is pyridyl, thiophenyl, oxazolyl, isoxazolyl, oxadiazolyl, thiazolyl, isothiazolyl or thiadiazolyl) or heterocyclyl (wherein heterocyclyl is tetrahydrofuranyl, [1,3]dioxolanyl, oxetanyl, thietanyl, 1-oxo-thietanyl or 1,1-dioxo-thietanyl), which themselves can be optionally substituted by one or two substituents independently selected from halogen, cyano, C₁-C₄alkyl, C₁-C₄haloalkyl, and C₁-C₄alkoxy), C₁-C₆haloalkyl (optionally substituted by one or two substituents independently selected from C₁-C₄-alkoxy, tri(C₁-C₄alkyl)silyloxy), and C₃-C₅alkenyl), C₁-C₆cyanoalkyl, C₁-C₆alkoxy(C₁-C₆)alkyl, C₁-C₆alkylcarbonyl(C₁-C₆)alkyl, C₁-C₆alkoxycarbonyl(C₁-C₆)alkyl, C₁-C₄alkylaminocarbonyl(C₁-C₆)alkyl, di(C₁-C₄alkyl)aminocarbonyl(C₁-C₆)alkyl, C₁-C₄haloalkylaminocarbonyl(C₁-C₆)alkyl, C₂-C₆alkenyloxycarbonyl(C₁-C₆)alkyl, C₃-C₆alkynyloxycarbonyl(C₁-C₃)alkyl, C₃-C₆cycloalkyl (optionally substituted by one or two substituents independently selected from C₁-C₄alkyl, and C₁-C₄haloalkyl, and, additionally, one of the ring member units can optionally represent C═O), C₃-C₆halocycloalkyl, C₄-C₇cycloalkenyl (optionally substituted by one or two substituents independently selected from C₁-C₄alkyl, and C₁-C₄haloalkyl, and, additionally, one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, phenyl(C₃-C₆)alkenyl, C₃-C₆alkynyl, C₃-C₆haloalkynyl, C₁-C₆alkoxy, heterocyclyl (wherein heterocyclyl is oxetanyl, 3-tetrahydrofuranyl-2-one, 1,1-dioxo-thietanyl, 1-oxo-thietanyl or thietanyl, and is optionally substituted by one or two substituents independently selected from halogen, cyano, C₁-C₄alkyl, C₁-C₄haloalkyl, and C₁-C₄alkoxy, and, additionally, a ring member unit can optionally represent C═O or C═NR⁵ where R⁵ is C₁-C₄ alkyl, or C₁-C₄ alkoxy); or R² represents the group “—C(R⁷)(R⁸)(R⁹)” wherein R⁷ is C₁-C₄alkyl, C₁-C₄haloalkyl, or cyclopropyl; R⁸ is hydrogen, C₁-C₄alkyl, C₁-C₄haloalkyl, or cyclopropyl, preferably hydrogen; and R⁹ is cyano, C₁-C₄alkyl, C₂-C₆alkenyl, C₂-C₆haloalkenyl, C₁-C₄alkoxy, C₂-C₅alkynyl, C₂-C₄alkoxycarbonyl, C₁-C₄alkylaminocarbonyl, di(C₁-C₃alkyl)aminocarbonyl, C₁-C₂haloalkylaminocarbonyl, C₃-C₆alkenyloxycarbonyl, C₃-C₄alkynyloxycarbonyl, or C₁-C₃alkylcarbonyl.

Embodiment 3 provides a method according to embodiment 1 or 2 wherein R¹ is F, Cl or Br.

Embodiment 4 provides a method according to any one of embodiments 1, 2 or 3 wherein R² is hydrogen, formyl, C₁-C₆alkyl (optionally substituted by heteroaryl (wherein heteroaryl is pyridyl, oxazolyl or oxadiazolyl) or heterocyclyl (wherein heterocyclyl is [1,3]dioxolanyl, thietanyl, 1-oxo-thietanyl, 1,1-dioxo-thietanyl or tetrahydrofuranyl), which themselves can be optionally substituted by one or two substituents independently selected from halogen, and C₁-C₄alkyl), C₁-C₆haloalkyl, C₁-C₃cyanoalkyl, C₁-C₄alkoxy(C₁-C₂)alkyl, C₁-C₃alkylcarbonyl(C₁-C₂)alkyl, C₁-C₄alkoxycarbonyl(C₁-C₂)alkyl, C₁-C₄alkylaminocarbonyl(C₁-C₂)alkyl, di(C₁-C₃alkyl)aminocarbonyl(C₁-C₂)alkyl, C₁-C₂haloalkylaminocarbonyl(C₁-C₂)alkyl, C₃-C₆alkenyloxycarbonyl(C₁-C₂)alkyl, C₃-C₄alkynyloxycarbonyl(C₁-C₂)alkyl, C₃-C₆cycloalkyl (optionally substituted by one or two C₁-C₄alkyl substituents and, additionally, one of the ring member units can optionally represent C═O), C₃-C₆halocycloalkyl, C₅-C₆cycloalkenyl (optionally substituted by one or two C₁-C₂alkyl substituents, and, additionally, one of the ring member units can optionally represent C═O), C₁-C₂alkyl-S(═O)n¹(C₁-C₄)alkyl where n¹ is 0, 1 or 2, C₃-C₅alkenyl, C₃-C₅haloalkenyl, C₃-C₆alkynyl; heterocyclyl (wherein heterocyclyl is 1,1-dioxo-thietanyl, 1-oxo-thietanyl or thietanyl) or R² represents the group “—C(R⁷)(R⁸)(R⁹)” wherein R⁷ is C₁-C₃alkyl; R⁸ is hydrogen, or C₁-C₃alkyl, preferably hydrogen; and R⁹ is cyano, C₂-C₄alkenyl, C₂-C₄haloalkenyl, C₂-C₄alkynyl, C₂-C₄alkoxycarbonyl, or C₃-C₆alkenyloxycarbonyl.

Embodiment 5 provides a method according to any one of embodiments 1, 2, 3 or 4 wherein R¹ is F or Br.

Embodiment 6 provides a method according to any one of embodiments 1, 2, 3, 4, or 5 wherein R² is hydrogen, formyl, C₁-C₄alkyl (optionally substituted by heterocyclyl, wherein heterocyclyl is [1,3]dioxolanyl, or tetrahydrofuranyl), C₁-C₆haloalkyl, C₁-C₃cyanoalkyl, C₁-C₂alkoxy(C₁-C₂)alkyl, C₁-C₃alkylcarbonyl(C₁-C₂)alkyl, C₁-C₄alkoxycarbonyl(C₁-C₂)alkyl, C₃-C₅alkenyloxycarbonyl(C₁-C₂)alkyl, C₃-C₄alkynyloxycarbonyl(C₁-C₂)alkyl, C₃-C₆cycloalkyl, C₁-C₂alkyl-S(═O)n¹(C₁-C₄)alkyl where n¹ is 0, C₃-C₅alkenyl, C₃-C₅haloalkenyl, C₃-C₆alkynyl; or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is cyano, C₂alkenyl, C₂alkynyl, C₂-C₄alkoxycarbonyl, or C₃-C₅alkenyloxycarbonyl.

Embodiment 7 provides a method according to any one of embodiments 1, 2, 3, 4, 5, or 6 wherein R² is hydrogen, formyl, C₁-C₄alkyl, C₁-C₆haloalkyl, C₁-C₂alkoxy(C₁-C₂)alkyl, C₁-C₄alkoxycarbonyl(C₁-C₂)alkyl, C₃cycloalkyl, C₁-C₂alkyl-S(═O)n¹(C₁-C₄)alkyl where n¹ is 0, C₃-C₅alkenyl, C₃-C₄haloalkenyl, C₃-C₆alkynyl; or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is cyano, C₂alkenyl, C₂alkynyl, C₂-C₄alkoxycarbonyl, or C₃-C₅alkenyloxycarbonyl.

Embodiment 8 provides a method according to any one of embodiments 1, 2, 3, 4, 5, 6, or 7 wherein R² is C₂-C₄alkyl, C₂-C₄haloalkyl, C₁-C₄alkoxycarbonyl(C₁-C₂)alkyl, C₁-C₂alkoxy(C₁-C₂)alkyl, C₁-C₂alkyl-S(═O)n¹(C₁-C₂)alkyl where n¹ is 0, C₃haloalkenyl, or C₃-C₆alkynyl.

Embodiment 9 provides a method according to any one of embodiments 1, 2, 3, 4, 5, 6, 7, or 8 wherein R¹ is F.

A preferred group of compounds are those of formula (I′) which are compounds of formula (I) wherein A is —CH₂—CH₂— or —CH═CH—; R¹ is halogen; and R² is hydrogen, formyl, cyano, hydroxy, NH₂, C₁-C₆alkyl (optionally substituted by aryl, aryloxy, heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl, C₁-C₄alkoxy(C₁-C₄)alkoxy(C₁-C₄)alkyl, C₁-C₆alkylcarbonyl(C₁-C₆)alkyl, C₁-C₄alkoxyimino(C₁-C₄)alkyl, C₁-C₄haloalkoxy(C₁-C₄)alkyl, C₁-C₆alkoxycarbonyl(C₁-C₆)alkyl, C₁-C₄alkoxy(C₁-C₄)alkoxycarbonyl(C₁-C₆)alkyl, hydroxycarbonyl(C₁-C₆)alkyl, aryloxycarbonyl(C₁-C₆)alkyl (wherein the aryl group can be optionally substituted by one or two substituents independently selected from halogen, cyano, nitro, C₁-C₄alkyl, C₁-C₄ haloalkyl, and C₁-C₄alkoxy), C₁-C₄alkylaminocarbonyl(C₁-C₆)alkyl, di(C₁-C₄alkyl)aminocarbonyl(C₁-C₆)alkyl, C₁-C₄haloalkylaminocarbonyl(C₁-C₆)alkyl, di(C₁-C₄haloalkyl)aminocarbonyl-C₁-C₆alkyl, C₁-C₂alkoxy(C₂-C₄)alkylaminocarbonyl(C₁-C₄)alkyl, C₂-C₆alkenyloxycarbonyl(C₁-C₆)alkyl, C₃-C₆alkynyloxycarbonyl(C₁-C₆)alkyl, (R³O)₂(O═)P(C₁-C₆)alkyl where R³ is hydrogen, C₁-C₄alkyl or benzyl, C₃-C₇cycloalkyl (optionally substituted by one to three substituents independently selected from C₁-C₄alkyl, C₁-C₄haloalkyl, and C₁-C₄alkoxy and, additionally, one of the ring member units can optionally represent C═O or C═NR⁴ where R⁴ is hydrogen, C₁-C₄alkyl, C₁-C₄haloalkyl, C₁-C₄cyanoalkyl, C₁-C₄alkoxy, or C₃-C₆cycloalkyl), C₃-C₇halocycloalkyl, C₃-C₇cycloalkenyl (optionally substituted by one or two substituents independently selected from C₁-C₄alkyl, and C₁-C₄haloalkyl, and, additionally, one of the ring member units can optionally represent C═O), C₃-C₇halocycloalkenyl, C₁-C₆alkyl-S(═O)n¹(C₁-C₆)alkyl where n¹ is 0, 1 or 2, C₁-C₆alkenyl, C₃-C₆haloalkenyl, aryl(C₃-C₆)alkenyl, C₃-C₆alkynyl, C₃-C₆haloalkynyl, aryl(C₃-C₆)alkynyl, C₃-C₆hydroxyalkynyl, C₁-C₆alkoxycarbonyl (optionally substituted by one to three substituents independently selected from halogen, hydroxy, cyano, C₁-C₄alkoxy, C₁-C₄haloalkyl, and aryl), aryloxycarbonyl (optionally substituted by one to three substituents independently selected from halogen, cyano, nitro, C₁-C₄alkyl, C₁-C₄haloalkyl, C₁-C₄alkoxy), C₃-C₆alkenyloxycarbonyl, C₃-C₆alkynyloxycarbonyl, C₁-C₆alkylcarbonyl, C₁-C₆haloalkylcarbonyl, aminocarbonyl, C₁-C₆alkylaminocarbonyl, di(C₁-C₆)alkylaminocarbonyl, aminothiocarbonyl, C₁-C₆alkylaminothiocarbonyl, di(C₁-C₆)alkylaminothiocarbonyl, C₁-C₆alkoxy, C₃-C₆alkenyloxy, C₃-C₈alkynyloxy, aryloxy (optionally substituted by one to three substituents independently selected from halogen, cyano, nitro, C₁-C₄alkyl, C₁-C₄haloalkyl, and C₁-C₄alkoxy), C₁-C₆alkylamino, di(C₁-C₆)alkylamino, C₃-C₆cycloalkylamino, C₁-C₄alkylthio, C₁-C₄alkylsulfinyl, C₁-C₄alkylsulfonyl, C₁-C₄haloalkylsulfonyl, aryl-S(═O)n² (optionally substituted by one or two substituents independently selected from halogen, nitro, and C₁-C₄alkyl) where n² is 0, 1 or 2, aryl (optionally substituted by one to three substituents independently selected from halogen, cyano, nitro, C₁-C₄alkyl, C₁-C₄haloalkyl, C₁-C₄alkoxy, and C₁-C₄haloalkoxy), heteroaryl (optionally substituted by one to three substituents independently selected from halogen, cyano, nitro, C₁-C₄alkyl, C₁-C₄haloalkyl, C₁-C₄alkoxy, and C₁-C₄haloalkoxy), heterocyclyl (optionally substituted by one to three substituents independently selected from halogen, cyano, nitro, C₁-C₄alkyl, C₁-C₄haloalkyl, C₁-C₄alkoxy, and C₁-C₄haloalkoxy, and, additionally, a ring member unit can optionally represent C═O or C═NR⁵ where R⁵ is hydrogen, C₁-C₄ alkyl, C₁-C₄ haloalkyl, C₁-C₄ cyanoalkyl, C₁-C₄ alkoxy, or C₃-C₆ cycloalkyl), (C₁-C₆alkylthio)carbonyl, (C₁-C₆alkylthio)thiocarbonyl, C₁-C₆alkyl-S(═O)n³(═NR⁶)—C₁-C₄alkyl wherein R⁶ is hydrogen, cyano, nitro, C₁-C₄alkyl and n³ is 0 or 1, or R² represents a group “—C(R⁷)(R⁸)(R⁹)” wherein R⁷ is C₁-C₄alkyl, C₁-C₄haloalkyl, or cyclopropyl, R⁸ is hydrogen, C₁-C₄alkyl, C₁-C₄haloalkyl, or cyclopropyl, preferably hydrogen, R⁹ is cyano, C₁-C₄alkyl, C₂-C₆alkenyl, C₂-C₆haloalkenyl, C₁-C₄alkoxy, C₂-C₅alkynyl, C₂-C₄alkoxycarbonyl, C₁-C₄alkylaminocarbonyl, di(C₁-C₃alkyl)aminocarbonyl, C₁-C₂haloalkylaminocarbonyl, C₃-C₆alkenyloxycarbonyl, C₃-C₄alkynyloxycarbonyl, or C₁-C₃alkylcarbonyl; or an agrochemically acceptable salt, N-oxide or isomer thereof.

Another preferred group of compounds are those of formula (IA) which are compounds of formula (I) wherein R¹ is F, Cl or Br; and R² is hydrogen, formyl, C₁-C₆alkyl (optionally substituted by phenyl, heteroaryl (wherein heteroaryl is pyridyl, thiophenyl, oxazolyl, isoxazolyl, oxadiazolyl, thiazolyl, isothiazolyl or thiadiazolyl) or heterocyclyl (wherein heterocyclyl is tetrahydrofuranyl, [1,3]dioxolanyl, oxetanyl, thietanyl, 1-oxo-thietanyl or 1,1-dioxo-thietanyl), which themselves can be optionally substituted by one or two substituents independently selected from halogen, cyano, C₁-C₄alkyl, C₁-C₄haloalkyl, and C₁-C₄alkoxy), C₁-C₆haloalkyl (optionally substituted by one or two substituents independently selected from C₁-C₄-alkoxy, tri(C₁-C₄alkyl)silyloxy), and C₃-C₅alkenyl), C₁-C₆cyanoalkyl, C₁-C₆alkoxy(C₁-C₆)alkyl, C₁-C₆alkylcarbonyl(C₁-C₆)alkyl, C₁-C₆alkoxycarbonyl(C₁-C₆)alkyl, C₁-C₄alkylaminocarbonyl(C₁-C₆)alkyl, di(C₁-C₄alkyl)aminocarbonyl(C₁-C₆)alkyl, C₁-C₄haloalkylaminocarbonyl(C₁-C₆)alkyl, C₂-C₆alkenyloxycarbonyl(C₁-C₆)alkyl, C₃-C₆alkynyloxycarbonyl(C₁-C₃)alkyl, C₃-C₆cycloalkyl (optionally substituted by one or two substituents independently selected from C₁-C₄alkyl, and C₁-C₄haloalkyl, and, additionally, one of the ring member units can optionally represent C═O), C₃-C₆halocycloalkyl, C₄-C₇cycloalkenyl (optionally substituted by one or two substituents independently selected from C₁-C₄alkyl, and C₁-C₄haloalkyl, and, additionally, one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, phenyl(C₃-C₆)alkenyl, C₃-C₆alkynyl, C₃-C₆haloalkynyl, C₁-C₆alkoxy, heterocyclyl (wherein heterocyclyl is oxetanyl, 3-tetrahydrofuranyl-2-one, 1,1-dioxo-thietanyl, 1-oxo-thietanyl or thietanyl, and is optionally substituted by one or two substituents independently selected from halogen, cyano, C₁-C₄alkyl, C₁-C₄haloalkyl, and C₁-C₄alkoxy, and, additionally, a ring member unit can optionally represent C═O or C═NR⁵ where R⁵ is C₁-C₄ alkyl, or C₁-C₄ alkoxy); or R² represents the group “—C(R⁷)(R⁸)(R⁹)” wherein R⁷ is C₁-C₄alkyl, C₁-C₄haloalkyl, or cyclopropyl; R⁸ is hydrogen, C₁-C₄alkyl, C₁-C₄haloalkyl, or cyclopropyl, preferably hydrogen; and R⁹ is cyano, C₁-C₄alkyl, C₂-C₆alkenyl, C₂-C₆haloalkenyl, C₁-C₄alkoxy, C₂-C₅alkynyl, C₂-C₄alkoxycarbonyl, C₁-C₄alkylaminocarbonyl, di(C₁-C₃alkyl)aminocarbonyl, C₁-C₂haloalkylaminocarbonyl, C₃-C₆alkenyloxycarbonyl, C₃-C₄alkynyloxycarbonyl, or C₁-C₃alkylcarbonyl; or an agrochemically acceptable salt, N-oxide or isomer thereof.

One group of compounds according to this embodiment are compounds of formula (IAA) which are compounds of formula (IA) wherein A is —CH₂—CH₂—.

Another group of compounds according to this embodiment are compounds of formula (IAB) which are compounds of formula (IA) wherein A is —CH═CH—.

A more preferred group of compounds are those of formula (IB) which are compounds of formula (I) wherein R¹ is Cl or Br; and R² is hydrogen, formyl, C₁-C₆alkyl (optionally substituted by heteroaryl (wherein heteroaryl is pyridyl, oxazolyl or oxadiazolyl) or heterocyclyl (wherein heterocyclyl is [1,3]dioxolanyl, thietanyl, 1-oxo-thietanyl, 1,1-dioxo-thietanyl or tetrahydrofuranyl), which themselves can be optionally substituted by one or two substituents independently selected from halogen, and C₁-C₄alkyl), C₁-C₆haloalkyl, C₁-C₃cyanoalkyl, C₁-C₄alkoxy(C₁-C₂)alkyl, C₁-C₃alkylcarbonyl(C₁-C₂)alkyl, C₁-C₄alkoxycarbonyl(C₁-C₂)alkyl, C₁-C₄alkylaminocarbonyl(C₁-C₂)alkyl, di(C₁-C₃alkyl)aminocarbonyl(C₁-C₂)alkyl, C₁-C₂haloalkylaminocarbonyl(C₁-C₂)alkyl, C₃-C₆alkenyloxycarbonyl(C₁-C₂)alkyl, C₃-C₄alkynyloxycarbonyl(C₁-C₂)alkyl, C₃-C₆cycloalkyl (optionally substituted by one or two C₁-C₄alkyl substituents and, additionally, one of the ring member units can optionally represent C═O), C₃-C₆halocycloalkyl, C₅-C₆cycloalkenyl (optionally substituted by one or two C₁-C₂alkyl substituents, and, additionally, one of the ring member units can optionally represent C═O), C₁-C₂alkyl-S(═O)n¹(C₁-C₄)alkyl where n¹ is 0, 1 or 2, C₃-C₅alkenyl, C₃-C₅haloalkenyl, C₃-C₆alkynyl; heterocyclyl (wherein heterocyclyl is 1,1-dioxo-thietanyl, 1-oxo-thietanyl or thietanyl) or R² represents the group “—C(R⁷)(R⁸)(R⁹)” wherein R⁷ is C₁-C₃alkyl; R⁸ is hydrogen, or C₁-C₃alkyl, preferably hydrogen; and R⁹ is cyano, C₂-C₄alkenyl, C₂-C₄haloalkenyl, C₂-C₄alkynyl, C₂-C₄alkoxycarbonyl, or C₃-C₆alkenyloxycarbonyl; or an agrochemically acceptable salt, N-oxide or isomer thereof.

One group of compounds according to this embodiment are compounds of formula (IBA) which are compounds of formula (IB) wherein A is —CH₂—CH₂—.

Another group of compounds according to this embodiment are compounds of formula (IBB) which are compounds of formula (IB) wherein A is —CH═CH—.

An even more preferred group of compounds are those of formula (IC) which are compounds of formula (I) wherein R¹ is Cl or Br; and R² is hydrogen, formyl, C₁-C₄alkyl (optionally substituted by heterocyclyl, wherein heterocyclyl is [1,3]dioxolanyl, or tetrahydrofuranyl), C₁-C₆haloalkyl, C₁-C₃cyanoalkyl, C₁-C₂alkoxy(C₁-C₂)alkyl, C₁-C₃alkylcarbonyl(C₁-C₂)alkyl, C₁-C₄alkoxycarbonyl(C₁-C₂)alkyl, C₃-C₅alkenyloxycarbonyl(C₁-C₂)alkyl, C₃-C₄alkynyloxycarbonyl(C₁-C₂)alkyl, C₃-C₆cycloalkyl, C₁-C₂alkyl-S(═O)n¹(C₁-C₄)alkyl where n¹ is 0, C₃-C₅alkenyl, C₃-C₅haloalkenyl, C₃-C₆alkynyl; or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is cyano, C₂alkenyl, C₂alkynyl, C₂-C₄alkoxycarbonyl, or C₃-C₅alkenyloxycarbonyl; or an agrochemically acceptable salt, N-oxide or isomer thereof.

One group of compounds according to this embodiment are compounds of formula (ICA) which are compounds of formula (IC) wherein A is —CH₂—CH₂—.

Another group of compounds according to this embodiment are compounds of formula (ICB) which are compounds of formula (IC) wherein A is —CH═CH—.

A yet more preferred group of compounds are those of formula (ID) which are compounds of formula (I) wherein R¹ is Cl or Br; and R² is hydrogen, formyl, C₁-C₄alkyl, C₁-C₆haloalkyl, C₁-C₂alkoxy(C₁-C₂)alkyl, C₁-C₄alkoxycarbonyl(C₁-C₂)alkyl, C₃cycloalkyl, C₁-C₂alkyl-S(═O)n¹(C₁-C₄)alkyl where n¹ is 0, C₃-C₅alkenyl, C₃-C₄haloalkenyl, C₃-C₆alkynyl; or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is cyano, C₂alkenyl, C₂alkynyl, C₂-C₄alkoxycarbonyl, or C₃-C₅alkenyloxycarbonyl; or an agrochemically acceptable salt, N-oxide or isomer thereof.

One group of compounds according to this embodiment are compounds of formula (IDA) which are compounds of formula (ID) wherein A is —CH₂—CH₂—.

Another group of compounds according to this embodiment are compounds of formula (IDB) which are compounds of formula (ID) wherein A is —CH═CH—.

An even more preferred group of compounds are those of formula (IE) which are compounds of formula (I) wherein R¹ is Cl or Br; and R² is C₂-C₄alkyl, C₂-C₄haloalkyl, C₁-C₄alkoxycarbonyl(C₁-C₂)alkyl, C₁-C₂alkoxy(C₁-C₂)alkyl, C₁-C₂alkyl-S(═O)n¹(C₁-C₂)alkyl where n¹ is 0, C₃haloalkenyl, or C₃-C₆alkynyl; or an agrochemically acceptable salt, N-oxide or isomer thereof.

One group of compounds according to this embodiment are compounds of formula (IEA) which are compounds of formula (IE) wherein A is —CH₂—CH₂—.

Another group of compounds according to this embodiment are compounds of formula (IEB) which are compounds of formula (IE) wherein A is —CH═CH—.

A most preferred group of compounds are those of formula (IF) which are compounds of formula (I) wherein R¹ is Cl or Br; and R² is C₃-C₄alkynyl; or an agrochemically acceptable salt, N-oxide or isomer thereof.

One group of compounds according to this embodiment are compounds of formula (IFA) which are compounds of formula (IF) wherein A is —CH₂—CH₂—.

Another group of compounds according to this embodiment are compounds of formula (IFB) which are compounds of formula (IF) wherein A is —CH═CH—.

Certain compounds of formula (I) are novel and as such form a further aspect of the invention.

For example, there are provided novel compounds of formula (IG) which are compounds of formula (I) wherein A is —CH₂—CH₂— or —CH═CH—; R¹ is F or I; and R² is C₁-C₅haloalkyl, C₃-C₅haloalkenyl, C₃-C₅haloalkynyl, C₃-C₅alkenyl, C₃-C₅alkynyl, C₃-C₇cycloalkyl, or C₅-C₇cycloalkenyl; or an agrochemically acceptable salt, N-oxide or isomer thereof.

Further, there are provided novel compounds of formula (IH) which are compounds of formula (I) wherein A is —CH₂—CH₂— or —CH═CH—; R¹ is F, Br or I; and R² is ethyl, propyl, isopropyl, 2-methylprop-2-enyl, C₁-C₂alkoxy(C₁-C₂)alkyl, C₁-C₂alkyl-S(═O)n⁵(C₁-C₂)alkyl where n⁵ is 0, 1 or 2 (preferably n⁵ is 0), C₃haloalkenyl, C₂-C₄alkoxycarbonyl(C₁-C₂)alkyl, or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is cyano, C₂alkenyl, C₂haloalkenyl, C₂alkynyl, C₂-C₄alkoxycarbonyl, or C₃-C₅alkenyloxycarbonyl; or, in addition, R² represents cyclobutyl, cyclopentenyl, cyclohexenyl, cycloheptenyl,

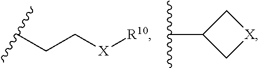

—CH₂C(R¹¹)═CH₂ or —CH₂CH═CH(R¹¹), where X is O, S, S(O) or S(O)₂, R¹⁰ is C₁-C₄alkyl, and R¹¹ is halogen or methyl; or an agrochemically acceptable salt, N-oxide or isomer thereof.

There are also provided novel compounds of formula (IH′) which are compounds of formula (I) wherein A is —CH₂—CH₂— or —CH═CH—; R¹ is Br; and R² is ethyl, propyl, isopropyl, 2-methylprop-2-enyl, C₁-C₂alkoxy(C₁-C₂)alkyl, C₁-C₂alkyl-S(═O)n⁵(C₁-C₂)alkyl where n⁵ is 0, 1 or 2 (preferably n⁵ is 0), C₃haloalkenyl, C₂-C₄alkoxycarbonyl(C₁-C₂)alkyl, or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is cyano, C₂alkenyl, C₂haloalkenyl, C₂alkynyl, C₂-C₄alkoxycarbonyl, or C₃-C₅alkenyloxycarbonyl; or an agrochemically acceptable salt, N-oxide or isomer thereof.

A preferred group of novel compounds are those of formula (IH′A) which are compounds of formula (IH′) wherein R² is 2-methylprop-2-enyl, C₁-C₂alkoxy(C₁-C₂)alkyl, C₁-C₂alkyl-S(═O)n⁵(C₁-C₂)alkyl where n⁵ is 0, C₃haloalkenyl, or R² represents the group “—C(R⁷)(R⁸)(R⁹)” wherein R7 is C₁-C₂alkyl; R8 is hydrogen or C₁-C₂alkyl, preferably hydrogen; and R9 is cyano, C₂alkenyl, C₂haloalkenyl, C₂alkynyl, C₂-C₄alkoxycarbonyl, or C₃-C₅alkenyloxycarbonyl; or an agrochemically acceptable salt, N-oxide or isomer thereof.

A more preferred group of novel compounds are those of formula (IH′B) which are compounds of formula (IH′) wherein R² is 2-methylprop-2-enyl, C₁-C₂alkoxy(C₁-C₂)alkyl, C₁-C₂alkyl-S(═O)n⁵(C₁-C₂)alkyl where n⁵ is 0, or R² represents the group “—C(R⁷)(R⁸)(R⁹)” wherein R7 is methyl; R8 is hydrogen or methyl, preferably hydrogen; and R9 is C₂haloalkenyl, C₂alkynyl, or C₂-C₄alkoxycarbonyl; or an agrochemically acceptable salt, N-oxide or isomer thereof.

An even more preferred group of novel compounds are those of formula (IH′C) which are compounds of formula (IH′) wherein R² is C₁-C₂alkoxy(C₁-C₂)alkyl, C₁-C₂alkyl-S(═O)n⁵(C₁-C₂)alkyl where n⁵ is 0, or R² represents the group “—C(R⁷)(R⁸)(R⁹)” wherein R7 is methyl; R8 is hydrogen or methyl, preferably hydrogen; and R9 is C₂haloalkenyl, or C₂alkynyl; or an agrochemically acceptable salt, N-oxide or isomer thereof.

A most preferred group of novel compounds are those of formula (IH′D) which are compounds of formula (IH′) wherein R² is C₁-C₂alkyl-S(═O)n⁵(C₁-C₂)alkyl where n⁵ is 0, or R² represents the group “—C(R⁷)(R⁸)(R⁹)” wherein R7 is methyl; R8 is hydrogen or methyl, preferably hydrogen; and R9 is or C₂alkynyl; or an agrochemically acceptable salt, N-oxide or isomer thereof.

Another group of novel compounds are those of formula (IJ) which are compounds of formula (I) wherein A is —CH₂—CH₂—; R¹ is Cl; and R² is propyl, 2-methylprop-2-enyl, C₁-C₂alkyl-S(═O)n⁵(C₁-C₂)alkyl where n⁵ is 0, 1 or 2 (preferably n⁵ is 0), or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is C₂alkenyl, C₂haloalkenyl, C₂alkynyl, or C₃-C₅alkenyloxycarbonyl; or, in addition, R² represents cyclobutyl, cyclopentenyl, cyclohexenyl, cycloheptenyl,

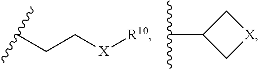

—CH₂C(R¹¹)═CH₂ or —CH₂CH═CH(R¹¹), where X is O, S, S(O) or S(O)₂, R¹⁰ is C₁-C₄alkyl, and R¹¹ is halogen or methyl; or an agrochemically acceptable salt, N-oxide or isomer thereof, provided that R² is not 2-methylbut-3-yn-2-yl.

Another group of novel compounds are those of formula (IJ′) which are compounds of formula (I) wherein A is —CH₂—CH₂—; R¹ is Cl; and R2 is propyl, 2-methylprop-2-enyl, C₁-C₂alkyl-S(═O)n⁵(C₁-C₂)alkyl where n⁵ is 0, 1 or 2 (preferably n⁵ is 0), or R² represents the group “—C(R⁷)(R⁹)(R⁹)” wherein R⁷ is C₁-C₂alkyl; R⁹ is hydrogen or C₁-C₂alkyl, preferably hydrogen; and R⁹ is C₂alkenyl, C₂haloalkenyl, C₂alkynyl, or C₃-C₅alkenyloxycarbonyl; or an agrochemically acceptable salt, N-oxide or isomer thereof, provided that R² is not 2-methylbut-3-yn-2-yl.

A preferred group compounds of formula (IJ′) are compounds of formula (ISA) wherein R² is 2-methylprop-2-enyl, C₁-C₂alkyl-S(═O)n⁵(C₁-C₂)alkyl where n⁵ is 0, or R² represents the group “—C(R⁷)(R⁹)(R⁹)” wherein R7 is C₁-C₂alkyl; R8 is hydrogen or C₁-C₂alkyl, preferably hydrogen; and R9 is C₂alkenyl, C₂haloalkenyl, or C₂alkynyl; or an agrochemically acceptable salt, N-oxide or isomer thereof, provided that R2 is not 2-methylbut-3-yn-2-yl.

A more preferred group compounds of formula (IJ′) are compounds of formula (IJ′B) wherein R² is C₁-C₂alkyl-S(═O)n⁵(C₁-C₂)alkyl where n⁵ is 0, or R² represents the group “—C(R⁷)(R⁹)(R⁹)” wherein R7 is methyl; R8 is hydrogen or methyl, preferably hydrogen; and R9 is C₂haloalkenyl, or C₂alkynyl; or an agrochemically acceptable salt, N-oxide or isomer thereof, provided that R2 is not 2-methylbut-3-yn-2-yl.

An even more preferred group compounds of formula (IJ′) are compounds of formula (IJ′C) wherein R² is C₁-C₂alkyl-S(═O)n⁵(C₁-C₂)alkyl where n⁵ is 0, or R² represents the group “—C(R⁷)(R⁹)(R⁹)” wherein R7 is methyl; R8 is hydrogen; and R9 is C₂haloalkenyl, or C₂alkynyl; or an agrochemically acceptable salt, N-oxide or isomer thereof.

A most preferred group compounds of formula (IJ′) are compounds of formula (IJ′D) wherein R² is C₁-C₂alkyl-S(═O)n⁵(C₁-C₂)alkyl where n⁵ is 0, or R² represents the group “—C(R⁷)(R⁹)(R⁹)” wherein R7 is methyl; R8 is hydrogen; and R9 is C₂alkynyl; or an agrochemically acceptable salt, N-oxide or isomer thereof.

Another group of novel compounds are those of formula (IK) which are compounds of formula (I) wherein A is —CH═CH—; R¹ is Cl; and R2 is ethyl, propyl, isopropyl, 2-methylprop-2-enyl, C₁-C₂alkoxy(C₁-C₂)alkyl, C₁-C₂alkyl-S(═O)n⁵(C₁-C₂)alkyl where n⁵ is 0, 1 or 2 (preferably n⁵ is 0), C₃haloalkenyl, C₂-C₄alkoxycarbonyl(C₁-C₂)alkyl, or R² represents the group “—C(R⁷)(R⁸)(R⁹)” wherein R⁷ is C₁-C₂alkyl; R⁸ is hydrogen or C₁-C₂alkyl, preferably hydrogen; and R9 is C₂alkenyl, C₂haloalkenyl, C₂alkynyl, C₂-C₄alkoxycarbonyl, or C₃-C₅alkenyloxycarbonyl; or, in addition, R² represents cyclobutyl, cyclopentenyl, cyclohexenyl, cycloheptenyl,

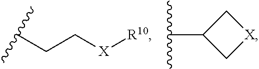

—CH₂C(R¹¹)═CH₂ or —CH₂CH═CH(R¹¹), where X is O, S, S(O) or S(O)₂, R¹⁰ is C₁-C₄alkyl, and R¹¹ is halogen or methyl; or an agrochemically acceptable salt, N-oxide or isomer thereof.

Another group of novel compounds are those of formula (IK′) which are compounds of formula (I) wherein A is —CH═CH—; R¹ is Cl; and R2 is ethyl, propyl, isopropyl, 2-methylprop-2-enyl, C₁-C₂alkoxy(C₁-C₂)alkyl, C₁-C₂alkyl-S(═O)n⁵(C₁-C₂)alkyl where n⁵ is 0, 1 or 2 (preferably n⁵ is 0), C₃haloalkenyl, C₂-C₄alkoxycarbonyl(C₁-C₂)alkyl, or R² represents the group “—C(R⁷)(R⁸)(R⁹)” wherein R7 is C₁-C₂alkyl; R8 is hydrogen or C₁-C₂alkyl, preferably hydrogen; and R9 is C₂alkenyl, C₂haloalkenyl, C₂alkynyl, C₂-C₄alkoxycarbonyl, or C₃-C₅alkenyloxycarbonyl; or an agrochemically acceptable salt, N-oxide or isomer thereof.

A preferred group compounds of formula (IK′) are compounds of formula (IK′A) wherein R² is 2-methylprop-2-enyl, C₁-C₂alkoxy(C₁-C₂)alkyl, C₁-C₂alkyl-S(═O)n⁵(C₁-C₂)alkyl where n⁵ is 0, C₃haloalkenyl, or R² represents the group “—C(R⁷)(R⁸)(R⁹)” wherein R7 is C₁-C₂alkyl; R8 is hydrogen or C₁-C₂alkyl, preferably hydrogen; and R9 is C₂alkenyl, C₂haloalkenyl, C₂alkynyl, or C₂-C₄alkoxycarbonyl; or an agrochemically acceptable salt, N-oxide or isomer thereof.

A more preferred group compounds of formula (IK′) are compounds of formula (IK′B) wherein R² is 2-methylprop-2-enyl, C₁-C₂alkyl-S(═O)n⁵(C₁-C₂)alkyl where n⁵ is 0, C₃haloalkenyl, or R² represents the group “—C(R⁷)(R⁸)(R⁹)” wherein R7 is methyl; R8 is hydrogen or methyl, preferably hydrogen; and R9 is C₂alkenyl, C₂haloalkenyl, or C₂alkynyl; or an agrochemically acceptable salt, N-oxide or isomer thereof.

An even more preferred group compounds of formula (IK′) are compounds of formula (IK′C) wherein R² is C₁-C₂alkyl-S(═O)n⁵(C₁-C₂)alkyl where n⁵ is 0, C₃haloalkenyl, or R² represents the group “—C(R⁷)(R⁸)(R⁹)” wherein R7 is methyl; R8 is hydrogen or methyl, preferably hydrogen; and R9 is C₂haloalkenyl, or C₂alkynyl; or an agrochemically acceptable salt, N-oxide or isomer thereof.

A most preferred group compounds of formula (IK′) are compounds of formula (IK′D) wherein R² is C₁-C₂alkyl-S(═O)n⁵(C₁-C₂)alkyl where n⁵ is 0, C₃haloalkenyl, or R² represents the group “—C(R⁷)(R⁸)(R⁹)” wherein R7 is methyl; R8 is hydrogen or methyl, preferably hydrogen; and R9 is C₂alkynyl; or an agrochemically acceptable salt, N-oxide or isomer thereof.

Certain novel compounds of formula (I) possess enhanced insecticidal properties and as such form a yet further aspect of the invention.

The tables below illustrate specific compounds of the invention.

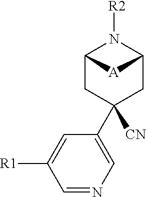

Table 1 provides 322 compounds of formula (I) wherein R1 is Cl, A=—CH₂—CH₂— and where the value of R2 is given in Table 1 (below).

TABLE 1

| Entry | R2 |
|---|---|
| 1.001 | CH₂C(O)OH |
| 1.002 | N′-nitrocarbamimidoyl |
| 1.003 | 2,2,2-trifluoroethyl |
| 1.004 | oxetan-3-yl |
| 1.005 | 1-oxothietan-3-yl |
| 1.006 | 1,1-dioxothietan-3-yl |
| 1.007 | 2-hydroxyethyl |
| 1.008 | 2-hydroxy-2-phenyl-ethyl |
| 1.009 | 2-(2-ethoxyethoxy)-1-methyl-2-oxo-ethyl |
| 1.010 | 2-(2-ethoxyethoxy)-1-acetoxy-2-oxo-ethyl |
| 1.011 | 1-methoxycarbonylbutyl |
| 1.012 | 1-ethoxycarbonylbutyl |
| 1.013 | 1-methoxycarbonylpentyl |
| 1.014 | 2-(2-methoxyethoxy)-2-oxo-ethyl |
| 1.015 | 2-isopropoxy-2-oxo-ethyl |
| 1.016 | 2-oxobutyl |
| 1.017 | 1-methylprop-2-ynyl |
| 1.018 | 2-methoxyethyl |
| 1.019 | but-3-ynyl |
| 1.020 | but-2-ynyl |
| 1.021 | isobutyl |
| 1.022 | N-cyano-C-methylsulfanyl-carbonimidoyl |
| 1.023 | 2-ethoxy-1-methyl-2-oxo-ethyl |
| 1.024 | 1,1-dimethylprop-2-ynyl |
| 1.025 | 1,2,4-oxadiazol-3-ylmethyl |
| 1.026 | 2-ethoxy-2-oxo-ethyl |
| 1.027 | tetrahydrofuran-3-ylmethyl |
| 1.028 | cyclobutylmethyl |
| 1.029 | oxetan-2-ylmethyl |
| 1.030 | 2-bromoallyl |
| 1.031 | thietan-3-yl |
| 1.032 | benzylsulfanyl |
| 1.033 | N′-cyano-N-methyl-carbamimidoyl |
| 1.034 | 2-oxotetrahydrofuran-3-yl |
| 1.035 | [2-(1-methoxy-4-piperidyl)-2-oxo-ethyl |
| 1.036 | 2-cyanoacetyl |
| 1.037 | 2-oxotetrahydrofuran-3-yl)oxycarbonyl |
| 1.038 | 4-toluenesulfonyl |
| 1.039 | 3-phenylprop-2-ynyl |
| 1.040 | 4-chlorobut-2-ynyl |
| 1.041 | pent-2-ynyl |
| 1.042 | propargyl |
| 1.043 | 2,2-difluoroethyl |
| 1.044 | 2-methylsulfanylethyl |
| 1.045 | C(O)OCH₂Ph |
| 1.046 | 2-allyloxy-1-methyl-2-oxo-ethyl |
| 1.047 | 2-methoxy-1-(methoxymethyl)-2-oxo-ethyl |
| 1.048 | 1-methyl-2-oxo-2-prop-2-ynoxy-ethyl) |
| 1.049 | 2-methoxy-1-methyl-2-oxo-ethyl |
| 1.050 | 4,4,4-trifluorobutyl |
| 1.051 | (E/Z)-3-chloroallyl |
| 1.052 | 2-methylallyl |
| 1.053 | 1,3-dioxolan-2-ylmethyl |
| 1.054 | 1-methyl-2-oxo-2-phenoxy-ethyl) |
| 1.055 | 2-methylsulfinylethyl |
| 1.056 | 3-fluoropropyl |
| 1.057 | isoxazole-4-carbonyl |
| 1.058 | isoxazole-5-carbonyl |
| 1.059 | 3-cyano-2-oxo-propanoyl |
| 1.060 | isoxazol-4-ylmethyl |
| 1.061 | (E)-2-cyano-3-oxo-prop-1-enyl |
| 1.062 | 2-cyano-3-oxo-propanoyl |
| 1.063 | 2-fluoroallyl |
| 1.064 | 2-(dimethylamino)-2-oxo-ethyl |
| 1.065 | 1-methyl-2-oxo-propyl |
| 1.066 | 2-(methylsulfonimidoyl)ethyl |
| 1.067 | 5-methyl-2-oxo-1,3-dioxol-4-yl)methyl |
| 1.068 | 2,2,2-trifluoroacetyl |
| 1.069 | ethoxycarbonyl |
| 1.070 | C(O)Ot-Bu |
| 1.071 | cyclopropyl |
| 1.072 | ethyl |
| 1.073 | formyl |
| 1.074 | hydrogen |
| 1.075 | isopropyl |
| 1.076 | n-hexyl |
| 1.077 | methylsulfanyl |
| 1.078 | phenylsulfanyl |
| 1.079 | 3-fluorophenyl |
| 1.080 | 4-fluorophenyl |
| 1.081 | acetyl |
| 1.082 | allyl |
| 1.083 | benzyl |
| 1.084 | pentafluorophenyl |
| 1.085 | CH₂C(O)Me |
| 1.086 | CH₂CH₂C(O)OH |
| 1.087 | CH₂CN |
| 1.088 | 3-chlorobenzyl |
| 1.089 | 2-bromobenzyl |
| 1.090 | 3-tolyl |
| 1.091 | 2-(1,3-dimethylbutoxy)-1-methyl-2-oxo-ethyl |
| 1.092 | 1-cyano-1-methyl-ethyl |
| 1.093 | 5-chloro-3-pyridyl |
| 1.094 | 3,5-dimethylisoxazol-4-yl)methyl |
| 1.095 | (2-amino-2-oxo-1-phenyl-ethyl |
| 1.096 | 2-cyano-2-hydroxy-1-methyl-1-phenyl-ethyl |
| 1.097 | 1,1-dimethylallyl |
| 1.098 | 1-methoxycarbonylpropyl |
| 1.099 | 1,3-benzodioxol-5-yl |
| 1.100 | 4-tBu benzyl |
| 1.101 | 2-anilino-1-methyl-2-oxo-ethyl |
| 1.102 | 2-isopropoxy-1-methyl-2-oxo-ethyl |
| 1.103 | 2-isobutoxy-1-methyl-2-oxo-ethyl |
| 1.104 | 2-fluoroethyl |
| 1.105 | (3-chloro-4-fluoro-phenyl)methyl |
| 1.106 | [3,5-bis(trifluoromethyl)phenyl]methyl |
| 1.107 | 4-(trifluoromethyl)pyridine-3-carbonyl]amino |
| 1.108 | 2,2,3,3,3-pentafluoropropyl |
| 1.109 | 2,2,3,3-tetrafluoropropyl |
| 1.110 | diisopropylcarbamoyl |
| 1.111 | (3-chlorophenyl)-cyano-methyl |
| 1.112 | 5-ethoxycarbonyl-2-furyl)methyl |
| 1.113 | 3-methoxy-2-methyl-3-oxo-propanoyl) |
| 1.114 | (4-fluoro-3-methyl-phenyl)methyl |
| 1.115 | (4-allyl-2,3,5,6-tetrafluoro-phenyl)methyl |
| 1.116 | 3,5,5-trimethylhexyl |
| 1.117 | CH₂CH₂OEt |
| 1.118 | 1-methyl-2-oxo-2-propoxy-ethyl |
| 1.119 | 9H-fluoren-9-ylmethoxycarbonyl |
| 1.120 | (R)-1-phenylethyl |
| 1.121 | (S)-1-phenylethyl |
| 1.122 | 6-chloropyrimidin-4-yl |
| 1.123 | 3-ethoxy-3-oxo-propyl |
| 1.124 | 3-fluoro-2-pyridyl |
| 1.125 | 2,2,3,3,4,4,5,5-octafluoropentyl |
| 1.126 | fluorocarbonyl |
| 1.127 | (3-chloro-4-fluorophenyl)carbamoyl |
| 1.128 | morpholine-4-carbonyl |
| 1.129 | 4-methoxyphenyl |
| 1.130 | 4,4-difluorobut-3-enylsulfanylcarbothioyl |
| 1.131 | [5-(2-oxo-3H-1,3,4-oxadiazol-5-yl)-2-furyl]methyl |
| 1.132 | CH₂C(O)OMe |
| 1.133 | 4-phenoxyphenyl |
| 1.134 | 2-naphthyl |
| 1.135 | (3-fluoro-4-methylphenyl)methyl |
| 1.136 | 2,2-difluorobutyl |
| 1.137 | (3-bromo-5-fluoro-phenyl)methyl |
| 1.138 | (2-fluoro-6-nitro-phenyl)methyl |
| 1.139 | 4-(2,6-dichlorobenzoyl) benzyl |
| 1.140 | 4-(2,2-dimethylpropanoyl)phenyl |
| 1.141 | 2,4,6-trifluorophenyl |
| 1.142 | (5-chloro-2-thienyl)methyl |
| 1.143 | (5-chlorothiadiazol-4-yl)methyl |
| 1.144 | 3-(chloromethyl)-1,2,4-thiadiazol-5-yl |
| 1.145 | 5-chlorothiazol-2-yl |
| 1.146 | 4-chlorophenyl |
| 1.147 | allyloxy |
| 1.148 | 4-(methylthio)phenyl |
| 1.149 | 2,2-difluoropropyl |
| 1.150 | 4,4,4-trifluoro-3-oxo-but-1-enyl |
| 1.151 | (4-isopropylphenyl)methyl |
| 1.152 | (2-fluoro-3-methyl-phenyl)methyl |
| 1.153 | 3-(4-fluorophenyloxy)benzyl |
| 1.154 | 2-t-butoxy-1-methyl-2-oxo-ethyl |
| 1.155 | 2,2,2-trichloroethoxycarbonyl |
| 1.156 | 2-chloroethoxycarbonyl |
| 1.157 | 4-fluorobenzoyl |
| 1.158 | cyano(phenyl)methyl |
| 1.159 | pentafluorobenzyl |
| 1.160 | 3-pyridylmethyl |
| 1.161 | 4-carboxyphenyl |
| 1.162 | 1-methyl-1-phenyl-allyl |
| 1.163 | 1-acetoxy-1-phenyl-ethyl |
| 1.164 | 1-(3-chlorophenyl)ethyl |
| 1.165 | 3-methoxyphenyl |
| 1.166 | 3-chlorophenyl |
| 1.167 | 1-methyl-2-morpholino-2-oxo-ethyl |
| 1.168 | (3,4-difluorophenyl)methyl |
| 1.169 | (3-phenyloxyphenyl)methyl |
| 1.170 | (4-bromophenyl)methyl |
| 1.171 | 2-phenoxyethyl |
| 1.172 | 1-methyl-2-oxo-2-sec-butoxy-ethyl |
| 1.173 | allyloxycarbonyl |
| 1.174 | (2,3,6-trifluorophenyl)methyl |
| 1.175 | (2,3-difluorophenyl)methyl |
| 1.176 | 3,3-difluoroallyl |
| 1.177 | 2-pyridylmethyl |
| 1.178 | methyl(phenyl)carbamoyl |
| 1.179 | (2-acetamidothiazol-4-yl)methyl |
| 1.180 | 4-trifluoromethylphenyl |
| 1.181 | 3,3,3-trifluoropropyl |
| 1.182 | (S)-CHMeC(O)OMe |
| 1.183 | pyrimidin-2-yl |
| 1.184 | [5-(hydrazinecarbonyl)-2-furyl]methyl |
| 1.185 | 3-(1,3-dioxolan-2-yl)phenyl |
| 1.186 | (E)-4,4,4-trifluoro-3-methoxy-but-2-enoyl |
| 1.187 | (4-fluoro-2-iodo-phenyl)methyl |
| 1.188 | (3-phenylphenyl)methyl |
| 1.189 | (2,3,4-triflurophenyl)methyl |
| 1.190 | 2-butoxy-1-methyl-2-oxo-ethyl |
| 1.191 | 4-cyanophenyl |
| 1.192 | (2-methylthiazol-4-yl)methyl |
| 1.193 | dithiocarboxy |
| 1.194 | 2,2,3,3,4,4,4-heptafluorobutyl |
| 1.195 | t-butylcarbamoyl |
| 1.196 | phosphonomethyl |
| 1.197 | t-butoxycarbonylamino |
| 1.198 | 2-carboxy-3,3,3-trifluoro-propyl |
| 1.199 | 3-t-butoxy-3-oxo-propyl |
| 1.200 | 3-trimethylsilylpropanoyl |
| 1.201 | (2-methylphenyl)methyl |
| 1.202 | (3-bromophenyl)methyl |
| 1.203 | 2-cyanoethyl |
| 1.204 | vinyloxycarbonyl |
| 1.205 | (3,5-dibromophenyl)methyl |
| 1.206 | isopropyloxycarbonyl |
| 1.207 | 4-pyridinylmethyl |
| 1.208 | 1-methyl-2-oxo-1-phenyl-ethyl |
| 1.209 | 2-(isopropylamino)-2-phenyl-propyl |
| 1.210 | 2-hydroxy-1-methyl-1-phenyl-ethyl |
| 1.211 | diethoxyphosphorylmethyl |
| 1.212 | 1-cyano-2-methyl-propyl |
| 1.213 | 2,2-difluoroethoxy |
| 1.214 | [ethoxycarbonyl- |
| | (ethoxycarbonylamino)amino]methyl |
| 1.215 | 3-vinylphenyl |
| 1.216 | (3,4-difluorophenyl)methyl |
| 1.217 | 3-methylphenyl |
| 1.218 | 3,3,3-trifluoro-2-methoxycarbonyl-propyl |
| 1.219 | (2-chlorophenyl)methyl |
| 1.220 | CN |
| 1.221 | C(O)OMe |
| 1.222 | C(O)OPh |
| 1.223 | methyl |
| 1.224 | nBu |
| 1.225 | NH₂ |
| 1.226 | OAc |
| 1.227 | OBn |
| 1.228 | OC(O)Ph |
| 1.229 | OMe |
| 1.230 | Ph |
| 1.231 | SO₂Ph |
| 1.232 | tBu |
| 1.233 | oxiran-2-ylmethyl |
| 1.234 | 2,2-dimethylbut-3-ynyl |
| 1.235 | CHMeCH₂CF₃ |
| 1.236 | oxetan-3-ylmethyl |
| 1.237 | 4-methoxybut-2-ynyl |
| 1.238 | pent-4-ynyl |
| 1.239 | 2-cyanoallyl |
| 1.240 | 2-methoxycarbonyloxyethyl |
| 1.241 | CH₂OC(O)Me |
| 1.242 | CH₂OC(O)iPr |
| 1.243 | CH₂OC(O)tBu |
| 1.244 | (2-oxotetrahydrofuran-3-yl)methyl |
| 1.245 | (5-oxotetrahydrofuran-2-yl)methyl |
| 1.246 | 2-[2-methoxyethyl(methyl)amino]-2-oxo-ethyl |
| 1.247 | 2-(2-methoxyethoxy)-1-methyl-2-oxo-ethyl |
| 1.248 | 2-(2-methoxyethoxy)-2-oxo-ethyl |
| 1.249 | (E)-cinnamyl |
| 1.250 | 2-methylsulfonylethyl |
| 1.251 | 1-methyl-2-methylsulfinyl-ethyl |
| 1.252 | CH₂S(O)₂Me |
| 1.253 | (2-nitrophenyl)sulfonyl |
| 1.254 | (CH₂)₂S(O)₂NHMe |
| 1.255 | CH₂S(O)₂NHMe |
| 1.256 | CH₂C(O)NHMe |
| 1.257 | thietan-3-ylmethyl |
| 1.258 | (1,1-dioxothietan-3-yl)methyl |
| 1.259 | (Z)-1-methylbut-2-enyl |
| 1.260 | 1-oxothietan-3-yl |
| 1.261 | 1,1-dioxothietan-3-yl |
| 1.262 | 1,1-dioxothiolan-3-yl |
| 1.263 | tetrahydrothiopyran-2-yl |
| 1.264 | tetrahydrothiophen-3-ylmethyl |
| 1.265 | tetrahydrothiophen-2-ylmethyl |
| 1.266 | 1,3-dithian-5-yl |
| 1.267 | tetrahydrofuran-2-yl |
| 1.268 | tetrahydropyran-2-yl |
| 1.269 | acetoxymethyl |
| 1.270 | 2-methylpropanoyloxymethyl |
| 1.271 | propanoyloxymethyl |
| 1.272 | 2-(2-hydroxy-5-oxo-2H-pyrrol-1-yl)ethyl |
| 1.273 | 2-(2-hydroxy-3,4-dimethyl-5-oxo-2H-pyrrol-1- |
| | yl)ethyl |
| 1.274 | 2-(2-hydroxy-5-oxo-pyrrolidin-1-yl)ethyl |
| 1.275 | 2-(2,2-difluoroethylamino)-2-oxo-ethyl |
| 1.276 | 2-(2-oxotetrahydrofuran-3-yl)ethyl |
| 1.277 | 2-(5-oxotetrahydrofuran-2-yl)ethyl |
| 1.278 | 3-methylbut-2-enyl |
| 1.279 | (Z)-2,3-dichloroallyl |
| 1.280 | (Z)-3-chlorobut-2-enyl |
| 1.281 | 3-oxocyclopenten-1-yl |
| 1.282 | 3-oxocyclohexen-1-yl |
| 1.283 | nPr |
| 1.284 | CH(CO₂Et)₂ |
| 1.285 | 1-methylallyl |
| 1.286 | CH(S) |
| 1.287 | 2,2,2-trifluoro-1-methyl-ethyl |
| 1.288 | (2-oxo-1,3-dioxolan-4-yl)methyl |
| 1.289 | 2-acetoxyethyl |
| 1.290 | [3-[(Z)-2-chloro-3,3,3-trifluoro-prop-1-enyl]-2,2- |
| | dimethyl-cyclopropyl]methyl |
| 1.291 | 2-methoxy-1-methyl-ethyl |
| 1.292 | 1-methyl-2-methylsulfanyl-ethyl |
| 1.293 | (E)-3-chloro-1-methyl-allyl |
| 1.294 | (Z)-3-chloro-1-methyl-allyl |
| 1.295 | (E)-1-methylbut-2-enyl |
| 1.296 | 2-(2-methoxyethoxy)ethyl |
| 1.297 | 2-chloroallyl |
| 1.298 | (2-methyl-5-oxo-tetrahydrofuran-3-yl) |
| 1.299 | tetrahydrofuran-2-ylmethyl |
| 1.300 | (Z)-3-chloroallyl |
| 1.301 | (S)-1-methylprop-2-ynyl |
| 1.302 | (R)-1-methylprop-2-ynyl |
| 1.303 | 2-ethoxycarbonyloxyethyl |
| 1.304 | 2-ethoxy-2-oxoethyl |
| 1.305 | 2-(2,5-dioxopyrrolidin-1-yl)ethyl |
| 1.306 | 2-benzyloxyethyl |
| 1.307 | 2-(cyclopropanecarbonyloxy)ethyl |
| 1.308 | 2-benzylsulfonylethyl |
| 1.309 | 2-oxo-2-phenylsulfanyl-ethyl |
| 1.310 | 2-benzylsulfanyl-2-oxo-ethyl |
| 1.311 | 2-benzylsulfanyl-1-methyl-2-oxo-ethyl |
| 1.312 | (2-methylpropanoylamino)methyl |
| 1.313 | 2-(ethylamino)-1-methyl-2-oxo-ethyl |
| 1.314 | 2-(methylamino)-1-methyl-2-oxo-ethyl |
| 1.315 | 2-(dimethylcarbamoyloxy)ethyl |
| 1.316 | 2-(2,5-dioxopyrrol-1-yl)ethyl |
| 1.317 | 2-(2-hydroxy-5-oxo-pyrrolidin-1-yl)ethyl |
| 1.318 | 2-(2-hydroxy-5-oxo-2H-pyrrol-1-yl)ethyl |
| 1.319 | 1-methoxycyclopropyl |
| 1.320 | cyclobutyl |
| 1.321 | tetrahydropyran-4-yl |
| 1.322 | 2-isopropoxy-2-oxo-methyl |

Table 2 provides 322 compounds of formula (I) wherein R1=Br, A=—CH₂—CH₂— and where the value of R2 is given in Table 1 (above).
Table 3 provides 322 compounds of formula (I) wherein R1=F, A=—CH₂—CH₂— and where the value of R2 is given in Table 1 (above).
Table 4 provides 322 compounds of formula (I) wherein R1=CI, A=—CH═CH— and where the value of R2 is given in Table 1 (above).
Table 5 provides 322 compounds of formula (I) wherein R1=Br, A=—CH═CH— and where the value of R2 is given in Table 1 (above).
Table 6 provides 322 compounds of formula (I) wherein R1=F, A=—CH═CH— and where the value of R2 is given in Table 1 (above).
Table 7 provides 322 compounds of formula (I) wherein R1=I, A=—CH₂—CH₂— and where the value of R2 is given in Table 1 (above).
Table 8 provides 322 compounds of formula (I) wherein R1=I, A=—CH═CH— and where the value of R2 is given in Table 1 (above).

Certain compounds disclosed in Tables 1 to 8 above are novel and as such form a further aspect of the invention.

The compounds of the invention may be made according to the procedures described in WO9637494 and WO9825924 or by a variety of methods as shown in the following schemes.

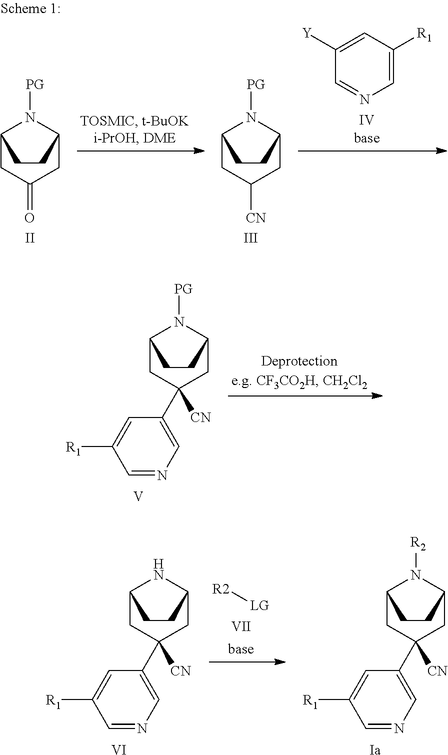

A compound of formula Ia, wherein A is —CH₂—CH₂— and the meanings of R₁ and R₂ are as defined above for compound of formula I, may be prepared according to Scheme 1.

A compound of formula II where PG is a protecting group, preferably a tert-butoxycarbonyl, ethoxycarbonyl or benzyloxycarbonyl group, may be prepared according to known procedures as reported in Tetrahedron, 2002, 58, 5669 or US2002198178. Compound II may be converted to the compound of formula III according to the known procedures described in W0199637494, WO2005058317 and J. Org. Chem. 1977, 42, 3114. The compound of formula III may then react with the compound of formula IV in the presence of a base such as NaN H₂, LDA or LiHMDS to give the compound of the formula V, wherein Y is independently selected from the group consisting of F, Cl, Br or I, preferably Y is F, Cl or Br and wherein R₁ is as defined for compound of formula I.

A compound of formula V may be transformed into the compound of formula VI by a deprotection reaction (for example, treatment with an acid, preferably 2,2,2-trifluoroacetic acid when PG is a tert-butoxycarbonyl group, see e.g. T. W. Greene et al. “Protective Groups in Organic Synthesis”, 3^{rd} edition 1999 by J. Wiley). A compound of formula VI may then react with the compound of formula VII, wherein LG is a leaving group such as CI, Br, I, OMs, OTs, OTf, in the presence of a base to give a compound of formula Ia, wherein A is —CH₂—CH₂— and the meanings of R₁ and R₂ are as defined as above for compound of formula I. Alternatively, compounds of formula Ia may be prepared using compounds of formula VI by reductive amination with the corresponding ketones in the presence of reducing agents such as NaB(CN)H₃ or NaBH(OAc)₃. In yet another alternative, compounds of formula Ia may be prepared via conjugate addition of compounds of formula VII to Michael acceptors such as vinylsulfones or enoates. Both methods are exemplified below.

The compound of the formula Ib, wherein A is —CH═CH— and the meanings of R₁ and R₂ are as defined as above, may be prepared according to the Scheme 2 using similar procedures as described for the preparation of the compound of formula Ia.

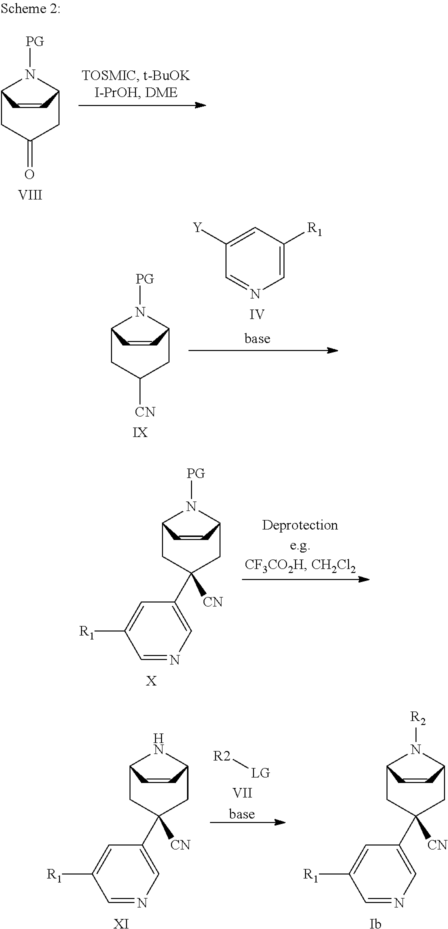

The compound of formula VIII, where PG is preferably a tert-butoxycarbonyl group or a carbonylbenzyloxy group, may be prepared according to the known procedures as shown in Scheme 3 (Tetrahedron Letters, 2002, 43, 1779, J. Org. Chem. 2003, 68, 8867 or Org. Lett. 2007, 9, 2871) or in Scheme 4 (Synlett, 14, 2003, 2175, J. Chem. Soc. Perkin Trans. I, 1992, 787-790). Details of the olefin metathesis reaction have been reported recently in Chem. Eur. J. 2012, 18, 8868 and Angew. Chem. 2000, 112, 3140.

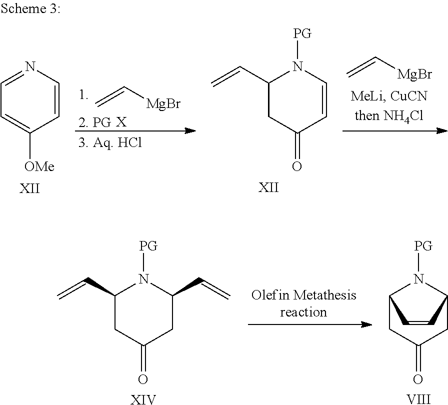

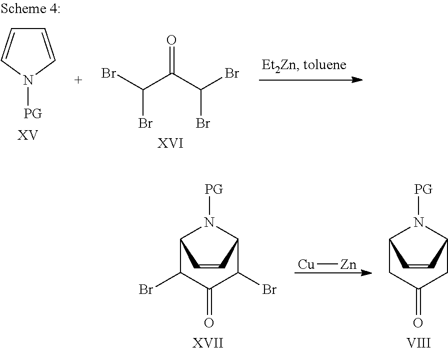

Certain intermediates of formula V, VI, IX, X and XI are novel and as such form a further aspect of the invention. For example, certain novel intermediates include compounds of formula V, VI, IX, X and XI wherein R1 and R2 (when present) are as defined in Tables 1 to 8 above.

Agrochemically acceptable salts of the compounds of formula I are, for example, acid addition salts. Those salts are formed, for example, with strong inorganic acids, such as mineral acids, for example perchloric acid, sulfuric acid, nitric acid, nitrous acid, a phosphoric acid or a hydrohalic acid, with strong organic carboxylic acids, such as unsubstituted or substituted, for example halogen-substituted, C₁-C₄ alkanecarboxylic acids, for example formic acid, acetic acid or trifluoroacetic acid, unsaturated or saturated dicarboxylic acids, for example oxalic, malonic, succinic, maleic, fumaric or phthalic acid, hydroxycarboxylic acids, for example ascorbic, lactic, malic, tartaric or citric acid, or benzoic acid, or with organic sulfonic acids, such as unsubstituted or substituted, for example halogen-substituted, C₁-C₄ alkane- or aryl-sulfonic acids, for example methane- or p-toluene-sulfonic acid.

In order to apply an active ingredient (i.e. a compound of formula (I)) to insects (in particular neonicotinoid resistant insects) and/or crops of useful plants as required by the methods of the invention said active ingredient may be used in pure form or, more typically, formulated into a composition which includes, in addition to said active ingredient, a suitable inert diluent or carrier and optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). SFAs include non-ionic, cationic and/or anionic surfactants, as well as surfactant mixtures. Examples are suitable phosphates, such as salts of the phosphoric ester of a p-nonylphenol/(4-14)ethylene oxide adduct, or phospholipids. Further suitable phosphates are tris-esters of phosphoric acid with aliphatic or aromatic alcohols and/or bis-esters of alkyl phosphonic acids with aliphatic or aromatic alcohols, which are a high performance oil-type adjuvant. These tris-esters have been described, for example, in WO0147356, WO0056146, EP-A-0579052 or EP-A-1018299 or are commercially available under their chemical name. Preferred tris-esters of phosphoric acid for use in the new compositions are tris-(2-ethylhexyl)phosphate, tris-n-octyl phosphate and tris-butoxyethyl phosphate, where tris-(2-ethylhexyl)phosphate is most preferred. Suitable bis-ester of alkyl phosphonic acids are bis-(2-ethylhexyl)-(2-ethylhexyl)-phosphonate, bis-(2-ethylhexyl)-(n-octyl)-phosphonate, dibutyl-butyl phosphonate and bis-(2-ethylhexyl)-tripropylene-phosphonate, where bis-(2-ethylhexyl)-(n-octyl)-phosphonate is particularly preferred.

The compositions according to the invention can preferably additionally include an additive comprising an oil of vegetable or animal origin, a mineral oil, alkyl esters of such oils or mixtures of such oils and oil derivatives. The amount of oil additive used in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil such as ADIGOR® and MERO®, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being important. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Also, alkoxylated fatty acids can be used as additives in the inventive compositions as well as polymethylsiloxane based additives, which have been described in WO08/037373.

Thus, in further embodiments according to any aspect of the invention mentioned hereinbefore, the compound of formula (I) will be in the form of a composition additionally comprising an agriculturally acceptable carrier or diluent.

It is preferred that all compositions (both solid and liquid formulations) comprise, by weight, 0.0001 to 95%, more preferably 1 to 85%, for example 5 to 60%, of a compound of formula (I). The composition is generally used for the control of pests such that a compound of formula (I) is applied at a rate of from 0.1 g to 10 kg per hectare, generally from 1 g to 6 kg per hectare, preferably 1 g to 2 kg per hectare, more preferably from 10 g to 1 kg per hectare, most preferably 10 g to 600 g per hectare.

When used in a seed dressing, a compound of formula (I) is generally used at a rate of 0.0001 g to 10 g (for example 0.001 g or 0.05 g), preferably 0.005 g to 10 g, more preferably 0.005 g to 4 g, per kilogram of seed.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallization in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurized, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerization stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I)).

Preferred compositions for use in methods of the invention are composed in particular of the following constituents (throughout, percentages are by weight):

Emulsifiable concentrates (EC):
active ingredient: 1 to 90%, preferably 5 to 20%
SFA: 1 to 30%, preferably 10 to 20%
solvent: 5 to 98%, preferably 70 to 85%

### Dusts (DP):

active ingredient: 0.1 to 10%, preferably 0.1 to 1%
solid carrier/diluent: 99.9 to 90%, preferably 99.9 to 99%
Suspension concentrates (SC):
active ingredient: 5 to 75%, preferably 10 to 50%
water: 94 to 24%, preferably 88 to 30%
SFA: 1 to 40%, preferably 2 to 30%
Wettable powders (WP):
active ingredient: 0.5 to 90%, preferably 1 to 80%, more preferably 20 to 30%
SFA: 0.5 to 20%, preferably 1 to 15%
solid carrier: 5 to 99%, preferably 15 to 98%

### Granules (GR, SG, WG):

active ingredient: 0.5 to 60%, preferably 5 to 60%, more preferably 50 to 60%
solid carrier/diluent: 99.5 to 40%, preferably 95 to 40%, more preferably 50 to 40%

A compound of formula I may be applied to a neonicotinoid resistant insect or crop of useful plants using any standard application method with which the skilled man is familiar, such as foliar spay or treatment of the plant propagation materials of the crop. Similarly, for methods of controlling insect resistance, neonicotinoid insecticides may be applied to an insect/crop/plant propagation material of useful plants using any known method of application. Further guidance may be found in the art, which includes for example, advice on application given on the labels of commercially available products.

In another aspect of the invention, the neonicotinoid insecticide is applied to the plant propagation material (such as seeds, young plants, transplants etc.) of the respective crops followed by the foliar application of a compound of the formula (I) starting in the 3- to 5-leaf up to the fruit setting crop stage. It has been found, that beginning with the 3- to 5-leaf crop stage, when the level of insect control by the neonicotinoid insecticide starts to decrease, another boost in insect control can be achieved by the foliar application of a compound of the formula (I), which, surprisingly, is accompanied by pronounced crop enhancement effects such as an increase in the formation of fine roots, synchronisation of flowering, drought resistance and, in particular, an increase in yield.

Examples of typical formulations are provided below (throughout, percentages are by weight)

| Example F1: Solutions | a) | b) | c) | d) |
|---|---|---|---|---|
| active ingredient | 80% | 10% | 5% | 95% |
| ethylene glycol monomethyl ether | 20% | — | — | — |
| polyethylene glycol (mol. wt 400) | — | 70% | — | — |
| N-methyl-2-pyrrolidone | — | 20% | — | — |
| epoxidised coconut oil | — | — | 1% | 5% |
| petroleum fraction (boiling range | — | — | 94% | — |
| 160-190.degree.) | | | | |

These solutions are suitable for application in the form of micro-drops.

| Example F2: Granules | a) | b) | c) | d) |
|---|---|---|---|---|
| active ingredient | 5% | 10% | 8% | 21% |
| Kaolin | 94% | — | 79% | 54% |
| Highly dispersed silicic acid | 1% | — | 13% | 7% |
| Attapulgite | — | 90% | — | 18% |

The active ingredient is dissolved in dichloromethane, the solution is sprayed onto the carrier, and the solvent is subsequently evaporated off in vacuo.

| Example F3: Dusts | a) | b) |
|---|---|---|
| active ingredient | 2% | 5% |
| Highly dispersed silicic acid | 1% | 5% |
| Talcum | 97% | — |
| Kaolin | — | 90% |

Ready-for-use dusts are obtained by intimately mixing the carriers with the active ingredient.

Example F4: Wettable powders

| | |
|---|---|
| active ingredient | 25% |
| Sodium sulphate | 5% |
| castor oil polyethylene glycol ether (36-37 mol of ethylene oxide) | 10% |
| silicone oil | 1% |
| Agridex | 2% |
| highly dispersed silicic acid | 10% |
| kaolin powder | 37% |
| sulfite spent lye powder | 5% |
| Ultravon W-300% (disodium salt of 1-benzyl-2 | 5% |
| heptadecylbenzimidazole-X,X′-disulfonic acid) | |

The active ingredient is mixed with the other formulation components and the mixture is ground in a suitable mill, affording wettable powders which can be diluted with water to give suspensions of the desired concentration.

| Example F5: Dusts | a) | b) |
|---|---|---|
| active ingredient | 5% | 8% |
| Talcum | 95% | — |
| Kaolin | — | 92% |

Ready-for-use dusts are obtained by mixing the active ingredient with the carrier and grinding the mixture in a suitable mill.

Example F6: Extruder granules

| | |
|---|---|
| active ingredient | 10% |
| Sodium lignosulfonate | 2% |
| Carboxymethylcellulose | 1% |
| Kaolin | 87% |

The active ingredient is mixed and ground with the other formulation components, and the mixture is subsequently moistened with water. The moist mixture is extruded and granulated and then the granules are dried in a stream of air.

Example F7: Coated granules

| | |
|---|---|
| active ingredient | 3% |
| Polyethylene glycol (mol. wt. 200) | 3% |
| Kaolin | 94% |

The finely ground active ingredient is uniformly applied, in a mixer, to the kaolin moistened with polyethylene glycol. Non-dusty coated granules are obtained in this manner.

Example F8: Suspension concentrate

| | |
|---|---|
| active ingredient | 40% |
| Ethylene glycol | 10% |
| Nonylphenol polyethylene glycol | 6% |
| Ether (15 mol of ethylene oxide) | |
| Sodium lignosulfonate | 10% |
| Carboxymethylcellulose | 1% |
| Aqueous formaldehyde solution (37%) | 0.2% |
| Aqueous silicone oil emulsion (75%) | 0.8% |
| Water | 32% |

The finely ground active ingredient is intimately mixed with the other formulation components giving a suspension concentrate from which suspensions of any desired concentration can be obtained by dilution with water.

| Example F9: Emulsifiable concentrates | a) | b) | c) |
|---|---|---|---|
| active ingredient | 25% | 40% | 50% |
| Calcium dodecylbenzenesulfonate | 5% | 8% | 6% |
| Castor oil polyethylene glycol ether (36 mol of | 5% | — | — |
| ethylene oxide) | | | |
| Tristyrylphenol polyethylene glycol ether (30 mol of | — | 12% | 4% |
| ethylene oxide | | | |
| Cyclohexanone | — | 15% | 20% |
| Xylene mixture | 65% | 25% | 20% |

Emulsions of any desired concentration can be produced from such concentrates by dilution with water.

| Example F10: Wettable powders | a) | b) | c) |
|---|---|---|---|
| active ingredient | 25% | 50% | 75% |
| Sodium lignosulfonate | 5% | 5% | — |
| Sodium laurylsulfate | 3% | — | 5% |
| Sodium diisobutylnapthalene-sulfonate | — | 6% | 10% |
| Octylphenol polyethylene glycol ether (7-8 mol of | — | 2% | — |
| ethylene oxide) | | | |
| Highly dispersed silicic acid | 5% | 10% | 10% |
| Kaolin | 62% | 27% | — |

The active ingredient is mixed with the other formulation components and the mixture is ground in a suitable mill, affording wettable powders which can be diluted with water to give suspensions of the desired concentration.

Example F11: Emulsifiable concentrate

| | |
|---|---|
| active ingredient | 10% |
| Octylphenol polyethylene glycol ether (4-5 mol of ethylene oxide) | 3% |
| Calcium dodecylbenzenesulfonate | 3% |
| Castor oil polyglycol ether (36 mol of ethylene oxide) | 4% |
| Cyclohexanone | 30% |
| Xylene mixture | 50% |

Emulsions of any required concentration can be obtained from this concentrate by dilution with water.

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example acetyltrimethyl ammonium bromide), imidazolines and amine salts.

Suitable anionic SFAs include alkali metals salts of fatty acids, salts of aliphatic monoesters of sulfuric acid (for example sodium lauryl sulfate), salts of sulfonated aromatic compounds (for example sodium dodecylbenzenesulfonate, calcium dodecylbenzenesulfonate, butylnaphthalene sulfonate and mixtures of sodium di-isopropyl- and tri-isopropyl-naphthalene sulfonates), ether sulfates, alcohol ether sulfates (for example sodium laureth-3-sulfate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulfosuccinamates, paraffin or olefine sulfonates, taurates and lignosulfonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapor or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilizers (for example nitrogen-, potassium- or phosphorus-containing fertilizers). Suitable formulation types include granules of fertilizer. The mixtures preferably contain up to 25% by weight of the compound of formula (I).

The invention therefore also provides a fertilizer composition comprising a fertilizer and a compound of formula (I).

The compositions of this invention may contain other compounds having biological activity, for example micronutrients or compounds having fungicidal activity or which possess plant growth regulating, herbicidal, insecticidal, nematicidal or acaricidal activity.

The compound of formula (I) may be the sole active ingredient of the composition or it may be admixed with one or more additional active ingredients such as a pesticide, e.g. a insecticide, fungicide or herbicide, or a synergist or plant growth regulator where appropriate. An additional active ingredient may provide a composition having a broader spectrum of activity or increased persistence at a locus; synergize the activity or complement the activity (for example by increasing the speed of effect or overcoming repellency) of the compound of formula (I); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin and gamma cyhalothrin), bifenthrin, fenpropathrin, cyfluthrin, tefluthrin, fish safe pyrethroids (for example ethofenprox), natural pyrethrin, tetramethrin, S-bioallethrin, fenfluthrin, prallethrin, acrinathirin, etofenprox or 5-benzyl-3-furylmethyl-(E)-(1R,3S)-2,2-dimethyl-3-(2-oxothiolan-3-ylidenemethyl)cyclopropane carboxylate;

b) Organophosphates, such as profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;

c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;

d) Benzoyl ureas, such as diflubenzuron, triflumuron, hexaflumuron, flufenoxuron, diafenthiuron, lufeneron, novaluron, noviflumuron or chlorfluazuron;

e) Organic tin compounds, such as cyhexatin, fenbutatin oxide or azocyclotin;

f) Pyrazoles, such as tebufenpyrad, tolfenpyrad, ethiprole, pyriprole, fipronil, and fenpyroximate;

g) Macrolides, such as avermectins or milbemycins, for example abamectin, emamectin benzoate, ivermectin, milbemycin, spinosad, azadirachtin, milbemectin, lepimectin or spinetoram;

h) Hormones or pheromones;

i) Organochlorine compounds, such as endosulfan (in particular alpha-endosulfan), benzene hexachloride, DDT, chlordane or dieldrin;

j) Amidines, such as chlordimeform or amitraz;

k) Fumigant agents, such as chloropicrin, dichloropropane, methyl bromide or metam;

l) Neonicotinoid compounds, such as imidacloprid, thiacloprid, acetamiprid, nitenpyram, dinotefuran, thiamethoxam, clothianidin, or nithiazine;

m) Diacylhydrazines, such as tebufenozide, chromafenozide or methoxyfenozide;

n) Diphenyl ethers, such as diofenolan or pyriproxifen;

o) Pyrazolines such as lndoxacarb or metaflumizone;

p) Ketoenols, such as Spirotetramat, spirodiclofen or spiromesifen;

q) Diamides, such as flubendiamide, chlorantraniliprole (Rynaxypyr®) or cyantraniliprole;

r) Essential oils such as Bugoil®—(PlantImpact); or

s) a compound selected from buprofezine, flonicamid, acequinocyl, bifenazate, cyenopyrafen, cyflumetofen, etoxazole, flometoquin, fluacrypyrim, fluensulfone, flufenerim, flupyradifuone, harpin, iodomethane, dodecadienol, pyridaben, pyridalyl, pyrimidifen, flupyradifurone, 4-[(6-Chloro-pyridin-3-ylmethyl)-(2,2-difluoro-ethyl)-amino]-5H-furan-2-one (DE 102006015467), CAS: 915972-17-7 (WO 2006129714; WO2011/147953; WO2011/147952), CAS: 26914-55-8 (WO 2007020986), chlorfenapyr, pymetrozine, sulfoxaflor and pyrifluqinazon.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulfonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704) (e.g. acibenzolar-S-methyl), alanycarb, aldimorph, anilazine, azaconazole, azoxystrobin, benalaxyl, benomyl, benthiavalicarb, biloxazol, bitertanol, bixafen, blasticidin S, boscalid, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulfate, copper tallate and Bordeaux mixture, cyclufenamid, cymoxanil, cyproconazole, cyprodinil, debacarb, di-2-pyridyl disulfide 1,1′-dioxide, dichlofluanid, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl-(Z)—N-benzyl-N-([methyl(methyl-thioethylideneaminooxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluopyram, fluoxastrobin, fluoroimide, fluquinconazole, flusilazole, flutolanil, flutriafol, fluxapyroxad, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, isopyrazam, kasugamycin, kresoxim-methyl, LY186054, LY211795, LY248908, mancozeb, mandipropamid, maneb, mefenoxam, metalaxyl, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, penflufen, penthiopyrad, phenazin oxide, phosetyl-AI, phosphorus acids, phthalide, picoxystrobin (ZA1963), polyoxinD, polyram, probenazole, prochloraz, procymidone, propamocarb, propiconazole, propineb, propionic acid, prothioconazole, pyrazophos, pyrifenox, pyrimethanil, pyraclostrobin, pyroquilon, pyroxyfur, pyrrolnitrin, quaternary ammonium compounds, quinomethionate, quinoxyfen, quintozene, sedaxane, sipconazole (F-155), sodium pentachlorophenate, spiroxamine, streptomycin, sulfur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram, N-[9-(dichloromethylene)-1,2,3,4-tetrahydro-1,4-methanonaphthalen-5-yl]-3-(difluoromethyl)-1-methyl-1H-pyrazole-4-carboxamide [1072957-71-1], 1-methyl-3-difluoromethyl-1H-pyrazole-4-carboxylic acid (2-dichloromethylene-3-ethyl-1-methyl-indan-4-yl)-amide, and 1-methyl-3-difluoromethyl-4H-pyrazole-4-carboxylic acid [2-(2,4-dichloro-phenyl)-2-methoxy-1-methyl-ethyl]-amide.

In addition, biological agents may be included in the composition of the invention e.g. Bacillus species such as Bacillus firmus, Bacillus cereus, Bacillus subtilis, and Pasteuria species such as Pasteuria penetrans and Pasteuria nishizawae. A suitable Bacillus firmus strain is strain CNCM 1-1582 which is commercially available as BioNem™. A suitable Bacillus cereus strain is strain CNCM 1-1562. Of both Bacillus strains more details can be found in U.S. Pat. No. 6,406,690. Other biological organisms that may be included in the compositions of the invention are bacteria such as Streptomyces spp. such as S. avermitilis, and fungi such as Pochonia spp. such as P. chlamydosporia. Also of interest are Metarhizium spp. such as M. anisopliae; Pochonia spp. such as P. chlamydosporia.

The compounds of formula (I) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™

The following mixtures of the compounds of formula I with active ingredients are preferred (the abbreviation “TX” means “one compound selected from the group consisting of the compounds described in Tables 1 to 8 (above) of the present invention”):

an adjuvant selected from the group of substances consisting of petroleum oils (alternative name) (628)+TX,
an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos (alternative name) [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate (alternative name)+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben (alternative name)+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin II (696)+TX, cinerins (696)+TX, clofentezine (158)+TX, closantel (alternative name) [CCN]+TX, coumaphos (174)+TX, crotamiton (alternative name) [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos (alternative name)+TX, dicofol (242)+TX, dicrotophos (243)+TX, dienochlor (1071)+TX, dimefox (1081)+TX, dimethoate (262)+TX, dinactin (alternative name) (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram (alternative name) [CCN]+TX, disulfoton (278)+TX, DNOC (282)+TX, dofenapyn (1113)+TX, doramectin (alternative name) [CCN]+TX, endosulfan (294)+TX, endothion (1121)+TX, EPN (297)+TX, eprinomectin (alternative name) [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad (alternative name)+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (alternative name) (473)+TX, isopropyl O-(methoxyaminothiophosphoryl)salicylate (IUPAC name) (473)+TX, ivermectin (alternative name) [CCN]+TX, jasmolin I (696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen (alternative name) [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime (alternative name) [CCN]+TX, mipafox (1293)+TX, monocrotophos (561)+TX, morphothion (1300)+TX, moxidectin (alternative name) [CCN]+TX, naled (567)+TX, NC-184 (compound code)+TX, NC-512 (compound code)+TX, nifluridide (1309)+TX, nikkomycins (alternative name) [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (alternative name) (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (alternative name) (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos (alternative name)+TX, selamectin (alternative name) [CCN]+TX, SI-0009 (compound code)+TX, sophamide (1402)+TX, spirodiclofen (738)+TX, spiromesifen (739)+TX, SSI-121 (development code) (1404)+TX, sulfiram (alternative name) [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam (alternative name)+TX, tetrachlorvinphos (777)+TX, tetradifon (786)+TX, tetranactin (alternative name) (653)+TX, tetrasul (1425)+TX, thiafenox (alternative name)+TX, thiocarboxime (1431)+TX, thiofanox (800)+TX, thiometon (801)+TX, thioquinox (1436)+TX, thuringiensin (alternative name) [CCN]+TX, triamiphos (1441)+TX, triarathene (1443)+TX, triazophos (820)+TX, triazuron (alternative name)+TX, trichlorfon (824)+TX, trifenofos (1455)+TX, trinactin (alternative name) (653)+TX, vamidothion (847)+TX, vaniliprole [CCN] and YI-5302 (compound code)+TX,
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,
an anthelmintic selected from the group of substances consisting of abamectin (1)+TX, crufomate (1011)+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX,
an avicide selected from the group of substances consisting of chloralose (127)+TX, endrin (1122)+TX, fenthion (346)+TX, pyridin-4-amine (IUPAC name) (23) and strychnine (745)+TX,
a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX,
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX,
a soil sterilant selected from the group of substances consisting of iodomethane (IUPAC name) (542) and methyl bromide (537)+TX,
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX,
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-yl (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B₂ (alternative name) (839)+TX, trimedlure C (alternative name) (839) and trunc-call (alternative name) [CCN]+TX,
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX,
an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion (alternative name) [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin (alternative name) [CCN]+TX, allyxycarb (866)+TX, alpha-cypermethrin (202)+TX, alpha-ecdysone (alternative name) [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (alternative name) (41)+TX, azamethiphos (42)+TX, azinphos-ethyl (44)+TX, azinphos-methyl (45)+TX, azothoate (889)+TX, Bacillus thuringiensis delta endotoxins (alternative name) (52)+TX, barium hexafluorosilicate (alternative name) [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (alternative name) (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate (alternative name)+TX, bromfenvinfos (914)+TX, bromocyclen (918)+TX, bromo-DDT (alternative name) [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben (alternative name)+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (alternative name) (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin 1 (696)+TX, cinerin 11 (696)+TX, cinerins (696)+TX, cis-resmethrin (alternative name)+TX, cismethrin (80)+TX, clocythrin (alternative name)+TX, cloethocarb (999)+TX, closantel (alternative name) [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton (alternative name) [CCN]+TX, crotoxyphos (1010)+TX, crufomate (1011)+TX, cryolite (alternative name) (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate (alternative name) [CCN]+TX, d-limonene (alternative name) [CCN]+TX, d-tetramethrin (alternative name) (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos (alternative name)+TX, dicresyl (alternative name) [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor (alternative name) [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin (alternative name) [CCN]+TX, DSP (1115)+TX, ecdysterone (alternative name) [CCN]+TX, EI 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin (alternative name) [CCN]+TX, esfenvalerate (302)+TX, etaphos (alternative name) [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (alternative name) (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad (alternative name)+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (alternative name) (473)+TX, isodrin (1235)+TX, isofenphos (1236)+TX, isolane (1237)+TX, isoprocarb (472)+TX, isopropyl O-(methoxy-aminothiophosphoryl)salicylate (IUPAC name) (473)+TX, isoprothiolane (474)+TX, isothioate (1244)+TX, isoxathion (480)+TX, ivermectin (alternative name) [CCN]+TX, jasmolin 1 (696)+TX, jasmolin 11 (696)+TX, jodfenphos (1248)+TX, juvenile hormone I (alternative name) [CCN]+TX, juvenile hormone II (alternative name) [CCN]+TX, juvenile hormone III (alternative name) [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (alternative name) (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (alternative name) (533)+TX, methoxychlor (534)+TX, methoxyfenozide (535)+TX, methyl bromide (537)+TX, methyl isothiocyanate (543)+TX, methylchloroform (alternative name) [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime (alternative name) [CCN]+TX, mipafox (1293)+TX, mirex (1294)+TX, monocrotophos (561)+TX, morphothion (1300)+TX, moxidectin (alternative name) [CCN]+TX, naftalofos (alternative name) [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron (alternative name) [CCN]+TX, pentachlorophenol (623)+TX, pentachlorophenyl laurate (IUPAC name) (623)+TX, permethrin (626)+TX, petroleum oils (alternative name) (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I (alternative name) [CCN]+TX, precocene II (alternative name) [CCN]+TX, precocene Ill (alternative name) [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia (alternative name) [CCN]+TX, quinalphos (711)+TX, quinalphos-methyl (1376)+TX, quinothion (1380)+TX, quintiofos (1381)+TX, R-1492 (development code) (1382)+TX, rafoxanide (alternative name) [CCN]+TX, resmethrin (719)+TX, rotenone (722)+TX, RU 15525 (development code) (723)+TX, RU 25475 (development code) (1386)+TX, ryania (alternative name) (1387)+TX, ryanodine (traditional name) (1387)+TX, sabadilla (alternative name) (725)+TX, schradan (1389)+TX, sebufos (alternative name)+TX, selamectin (alternative name) [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (alternative name) (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam (alternative name)+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox (alternative name)+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin (alternative name) [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron (alternative name)+TX, trichlorfon (824)+TX, trichlormetaphos-3 (alternative name) [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (alternative name) (725)+TX, veratrine (alternative name) (725)+TX, XMC (853)+TX, xylylcarb (854)+TX, Y1-5302 (compound code)+TX, zeta-cypermethrin (205)+TX, zetamethrin (alternative name)+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen[560121-52-0]+TX, cyflumetofen[400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole[704886-18-0]+TX, meperfluthrin [915288-13-0]+TX, tetramethylfluthrin [84937-88-2]+TX, triflumezopyrim (disclosed in WO 2012/092115)+TX,
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole[394730-71-3]+TX,
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone[318290-98-1]+TX,
a nitrification inhibitor selected from the group of substances consisting of potassium ethylxanthate [CCN] and nitrapyrin (580)+TX, a plant activator selected from the group of substances consisting of acibenzolar (6)+TX, acibenzolar-S-methyl (6)+TX, probenazole (658) and Reynoutria sachalinensis extract (alternative name) (720)+TX,
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,
a virucide selected from the group of substances consisting of imanin (alternative name) [CCN] and ribavirin (alternative name) [CCN]+TX,
a wound protectant selected from the group of substances consisting of mercuric oxide (512)+TX, octhilinone (590) and thiophanate-methyl (802)+TX,
and biologically active compounds selected from the group consisting of azaconazole (60207-31-0]+TX, bitertanol[70585-36-3]+TX, bromuconazole[116255-48-2]+TX, cyproconazole[94361-06-5]+TX, difenoconazole[119446-68-3]+TX, diniconazole [83657-24-3]+TX, epoxiconazole[106325-08-0]+TX, fenbuconazole[114369-43-6]+TX, fluquinconazole[136426-54-5]+TX, flusilazole[85509-19-9]+TX, flutriafol[76674-21-0]+TX, hexaconazole[79983-71-4]+TX, imazalil [35554-44-0]+TX, imibenconazole [86598-92-7]+TX, ipconazole[125225-28-7]+TX, metconazole[125116-23-6]+TX, myclobutanil [88671-89-0]+TX, pefurazoate[101903-30-4]+TX, penconazole[66246-88-6]+TX, prothioconazole[178928-70-6]+TX, pyrifenox[88283-41-4]+TX, prochloraz [67747-09-5]+TX, propiconazole[60207-90-1]+TX, simeconazole[149508-90-7]+TX, tebuconazole[107534-96-3]+TX, tetraconazole[112281-77-3]+TX, triadimefon[43121-43-3]+TX, triadimenol[55219-65-3]+TX, triflumizole[99387-89-0]+TX, triticonazole [131983-72-7]+TX, ancymidol[12771-68-5]+TX, fenarimol[60168-88-9]+TX, nuarimol [63284-71-9]+TX, bupirimate[41483-43-6]+TX, dimethirimol[5221-53-4]+TX, ethirimol [23947-60-6]+TX, dodemorph [1593-77-7]+TX, fenpropidine[67306-00-7]+TX, fenpropimorph [67564-91-4]+TX, spiroxamine[118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl[57646-30-7]+TX, metalaxyl[57837-19-1]+TX, R-metalaxyl [70630-17-0]+TX, ofurace [58810-48-3]+TX, oxadixyl[77732-09-3]+TX, benomyl [17804-35-2]+TX, carbendazim [10605-21-7]+TX, debacarb[62732-91-6]+TX, fuberidazole[3878-19-1]+TX, thiabendazole[148-79-8]+TX, chlozolinate[84332-86-5]+TX, dichlozoline[24201-58-9]+TX, iprodione[36734-19-7]+TX, myclozoline[54864-61-8]+TX, procymidone[32809-16-8]+TX, vinclozoline[50471-44-8]+TX, boscalid [188425-85-6]+TX, carboxin[5234-68-4]+TX, fenfuram [24691-80-3]+TX, flutolanil [66332-96-5]+TX, mepronil [55814-41-0]+TX, oxycarboxin[5259-88-1]+TX, penthiopyrad [183675-82-3]+TX, thifluzamide[130000-40-7]+TX, guazatine[108173-90-6]+TX, dodine[2439-10-3] [112-65-2] (free base)+TX, iminoctadine[13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl[143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol[2425-06-1]+TX, captan[133-06-2]+TX, dichlofluanid [1085-98-9]+TX, fluoroimide[41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper[53988-93-5]+TX, oxine-copper [10380-28-6]+TX, dinocap [131-72-6]+TX, nitrothal-isopropyl[10552-74-6]+TX, edifenphos [17109-49-8]+TX, iprobenphos [26087-47-8]+TX, isoprothiolane[50512-35-1]+TX, phosdiphen[36519-00-3]+TX, pyrazophos [13457-18-6]+TX, tolclofos-methyl [57018-04-9]+TX, acibenzolar-S-methyl[135158-54-2]+TX, anilazine[101-05-3]+TX, benthiavalicarb[413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone[117-80-6]+TX, diclocymet [139920-32-4]+TX, diclomezine[62865-36-5]+TX, dicloran[99-30-9]+TX, diethofencarb [87130-20-9]+TX, dimethomorph [110488-70-5]+TX, SYP-L190 (Flumorph) [211867-47-9]+TX, dithianon[3347-22-6]+TX, ethaboxam [162650-77-3]+TX, etridiazole[2593-15-9]+TX, famoxadone[131807-57-3]+TX, fenamidone[161326-34-7]+TX, fenoxanil [115852-48-7]+TX, fentin [668-34-8]+TX, ferimzone[89269-64-7]+TX, fluazinam [79622-59-6]+TX, fluopicolide[239110-15-7]+TX, flusulfamide[106917-52-6]+TX, fenhexamid[126833-17-8]+TX, fosetyl-aluminium[39148-24-8]+TX, hymexazol[10004-44-1]+TX, iprovalicarb[140923-17-7]+TX, IKF-916 (Cyazofamid) [120116-88-3]+TX, kasugamycin [6980-18-3]+TX, methasulfocarb[66952-49-6]+TX, metrafenone[220899-03-6]+TX, pencycuron[66063-05-6]+TX, phthalide[27355-22-2]+TX, polyoxins [11113-80-7]+TX, probenazole[27605-76-1]+TX, propamocarb[25606-41-1]+TX, proquinazid [189278-12-4]+TX, pyroquilon[57369-32-1]+TX, quinoxyfen[124495-18-7]+TX, quintozene[82-68-8]+TX, sulfur[7704-34-9]+TX, tiadinil [223580-51-6]+TX, triazoxide [72459-58-6]+TX, tricyclazole[41814-78-2]+TX, triforine[26644-46-2]+TX, validamycin [37248-47-8]+TX, zoxamide (RH7281) [156052-68-5]+TX, mandipropamid[374726-62-2]+TX, isopyrazam [881685-58-1]+TX, sedaxane[874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (dislosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate[915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide[926914-55-8]+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula I selected from Tables 1 to 8 (above) with active ingredients described above comprises a compound selected from Tables 1 to 8 (above) and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables 1 to 8 (above) and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables 1 to 8 (above) and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is preferably 1 to 2000 g of active ingredient per hectare, more preferably 10 to 1000 g/ha, most preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

### EXAMPLES

The following Examples illustrate, but do not limit, the invention.

The following abbreviations were used in this section: DMF: dimethylformamide; THF: tetrahydrofuran; EtOAc: ethyl acetate; s=singlet; bs=broad singlet; d=doublet; dd=double doublet; dt=double triplet; t=triplet, tt=triple triplet, q=quartet, sept=septet; m=multiplet; Me=methyl; Et=ethyl; Pr=propyl; Bu=butyl; M.p.=melting point; RT=retention time, [M+H]⁺=molecular mass of the molecular cation, [M−H]⁻=molecular mass of the molecular anion.

The following LC-MS methods were used to characterize the compounds:

Method A:

Spectra were recorded on a Triple Quad mass spectrometer from Agilent (series 6410) equipped with an electrospray source (Polarity: positive or negative ions, Capillary: 4.00 kV, Cone range: 30-60 V, Extractor: 2.00 V, Source Temperature: 350° C., Desolvation Temperature: 250° C., Cone Gas Flow: 11 L/Hr, Desolvation Gas Flow: 400 L/Hr, Mass range: 110 to 1000 Da) and an Agilent 1200 LC (Solvent degasser, quaternary pump, autosampler, Agilent 1260: heated column compartment and diode-array detector. Column: Xterra C18, 3.5 μm, 30×4.6 mm, Temp: 30° C., DAD Wavelength range (nm): 190 to 400, Solvent Gradient: A=water+0.1% HCOOH, B=Acetonitrile+0.1% HCOOH: gradient: 0 min 10% B; 2-3 min 100% B; 3.2 min 10% B; 4 min: 10% B. Flow (ml/min) 1.8.

Method B:

Spectra were recorded on a ZQ Mass Spectrometer from Waters (Single quadrupole mass spectrometer) equipped with an electrospray source (Polarity: positive or negative ions, Capillary: 3.00 kV, Cone range: 30-60 V, Extractor: 2.00 V, Source Temperature: 100° C., Desolvation Temperature: 250° C., Cone Gas Flow: 50 L/Hr, Desolvation Gas Flow: 400 L/Hr, Mass range: 100 to 900 Da) and an Agilent 1100 LC (Solvent degasser, binary pump, heated column compartment and diode-array detector. Column: Phenomenex Gemini C18, 3 μm, 30×3 mm, Temp: 60° C., DAD Wavelength range (nm): 210 to 500, Solvent Gradient: A=water+5% MeOH+0.05% HCOOH, B=Acetonitrile+0.05% HCOOH: gradient: 0 min 0% B; 2-2.8 min 100% B; 2.9-3 min 0%. Flow (ml/min) 1.7

Method C

ACQUITY SQD Mass Spectrometer from Waters (Single quadrupole mass spectrometer)

Ionisation method: Electrospray
Polarity: positive ions

### Capillary (kV) 3.00, Cone (V) 20.00, Extractor (V) 3.00, Source Temperature (° C.) 150,

### Desolvation Temperature (° C.) 400, Cone Gas Flow (L/Hr) 60, Desolvation Gas Flow (L/Hr) 700

Mass range: 100 to 800 Da
DAD Wavelength range (nm): 210 to 400
Method Waters ACQUITY UPLC with the following HPLC gradient conditions
(Solvent A: Water/Methanol 9:1, 0.1% formic acid and Solvent B: Acetonitrile, 0.1% formic acid)

| Time (minutes) | A (%) | B (%) | Flow rate (ml/min) |
|---|---|---|---|
| 0 | 100 | 0 | 0.75 |
| 2.5 | 0 | 100 | 0.75 |
| 2.8 | 0 | 100 | 0.75 |
| 3.0 | 100 | 0 | 0.75 |

Type of column: Waters ACQUITY UPLC HSS T3; Column length: 30 mm; Internal diameter of column: 2.1 mm; Particle Size: 1.8 micron; Temperature: 60° C.

Method D

MS ZMD Mass Spectrometer from Waters (Single quadrupole mass spectrometer)
Instrument Parameter: Ionisation method: Electrospray, polarity: positive (negative) ions; capillary (kV) 3.80, Cone (V) 30.00, Extractor (V) 3.00, Source Temperature (° C.) 150, Desolvation Temperature (° C.) 350, Cone Gas Flow (L/h) OFF, Desolvation Gas Flow (L/h) 600, mass range: 100 to 900 Da
   LC HP 1100 HPLC from Agilent: solvent degasser, binary pump, heated column compartment and diode-array detector.
Column: Phenomenex Gemini C18, 3 μm, 30×3 mm,
Temp: 60° C.
DAD Wavelength range (nm): 200 to 500
Solvent Gradient:
A=water+0.05% HCOOH
B=Acetonitrile/Methanol (4:1, v:v)+0.04% HCOOH

| Time (min) | A % | B % | Flow (mL/min) |
|---|---|---|---|
| 0.00 | 95.0 | 5.0 | 1.700 |
| 2.00 | 0.00 | 100.0 | 1.700 |
| 2.80 | 0.00 | 100.0 | 1.700 |
| 2.90 | 95.0 | 5.0 | 1.700 |
| 3.00 | 95.0 | 5.0 | 1.700 |

Method E:

Spectra were recorded on a SQD Mass Spectrometer from Waters (Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 250° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Phenomenex Gemini C18, 3 μm, 30×2 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 1.2-1.5 min 100% B; Flow (ml/min) 0.85

Method F:

Spectra were recorded on a SQD Mass Spectrometer from Waters (Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 250° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Phenomenex Gemini C18, 3 μm, 30×2 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 3.4-4.1 min 100% B; Flow (ml/min) 0.85

Method G:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 1.2-1.5 min 100% B; Flow (ml/min) 0.85

Method H:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 2.7-3.0 min 100% B; Flow (ml/min) 0.85

Method I:

Spectra were recorded on a Mass Spectrometer from Thermo equipped with an APCI source (Polarity: positive or negative ions, Capillary: - kV, Cone range: 50 V, Extractor: - V, Source Temperature: 400° C., Mass range: 100 to 1000 Da) and an LC from Thermo: Binary pump, heated column compartment and diode-array detector. Solvent degasser, binary pump, heated column compartment and diode-array detector. Column: Waters Xterra MS C18, 3.5 μm, 30×4.6 mm, Temp: 30° C., DAD Wavelength range (nm): 200 to 350, Solvent Gradient: A=Water with 0.05% HCOOH, B=MeOH. Gradient: 0 min 90% A, 4 min 100% B, 7.5 min 100% B, 7.8 min 90% A, 9 min 90% A.

### PREPARATION EXAMPLES

### Example C1

### Preparation of (1S,5R)-3-(5-bromo-pyridin-3-yl)-3-cyano-8-aza-bicyclo[3.2.1]octane-8-carboxylic acid tert-butyl ester (Compound 2.070)

### Step 1: Preparation of (1S,5R)-3-cyano-8-aza-bicyclo[3.2.1]octane-8-carboxylic acid tert-butyl ester

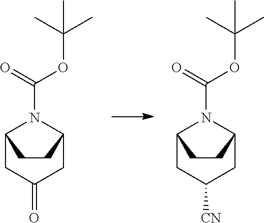 [SMILES: CC(C)(C)OC(=O)N1[C@@H]2CC[C@H]1CC(=O)C2 ; CC(C)(C)OC(=O)N1[C@@H]2CC[C@H]1C[C@H](C#N)C2]

6.23 g (55.5 mmol) potassium t-butoxide were suspended at 0° C. in 15 mL 1,2-dimethoxyethane (DME) under argon. Then, within 30 min, 6.50 g (33.3 mmol) tosylmethyl isocyanide dissolved in 20 mL DME were added dropwise keeping the temperature below 5° C. The reaction mixture became immediately brown and was stirred for additional 1 h at 0° C. Then 3.4 mL (44.6 mmol) iso-propanol were added dropwise at 0° C. The reaction mixture was stirred for additional 30 min, then 5.00 g (22.2 mmol) (1S,5R)-3-oxo-8-azabicyclo[3.2.1]octane-8-carboxylic acid tert-butyl ester (prepared according to Berdini et al., Tetrahedron 2002, 58, 5669) were added dropwise within 30 minutes maintaining the reaction temperature below 5° C. The reaction mixture is stirred 1 h at 0° C. after addition was complete and then allowed to warm to room temperature overnight. The reaction mixture is filtered over Celite (to remove potassium p-toluenesulfinate) and the residue intensively washed with solvent. The organic layers were combined and evaporated to give the crude product. The crude material was purified by flash chromatography (ethyl acetate/cyclohexane) to afford (1S,5R)-3-cyano-8-aza-bicyclo[3.2.1]octane-8-carboxylic acid tert-butyl ester as a white solid (m.p. 97-98° C.).

¹H NMR (CDCl₃, TMS) δ/ppm: 1.48 (s, 9H), 1.62 (m, 2H), 1.85 (m, 2H), 1.95-2.10 (br m, 4H), 2.90-3.05 (m, 1H), 4.15-4.35 (br s, 2H).

### Step 2: Preparation of (1S,5R)-3-(5-bromo-pyridin-3-yl)-3-cyano-8-aza-bicyclo[3.2.1]octane-8-carboxylic acid tert-butyl ester (Compound 2.070)

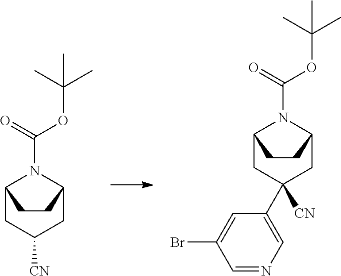 [SMILES: CC(C)(C)OC(=O)N1[C@@H]2CC[C@H]1C[C@H](C#N)C2 ; CC(C)(C)OC(=O)N1[C@@H]2CC[C@H]1C[C@](C#N)(c1cncc(Br)c1)C2]

46.75 mL (1 M solution in tetrahydrofuran) Lithium bis(trimethylsilyl)amide was added dropwise to a stirred solution of 10.04 g (42.5 mmol) (1S,5R)-3-cyano-8-aza-bicyclo[3.2.1]octane-8-carboxylic acid tert-butyl ester and 7.85 g (44.62 mmol) 3-bromo-5-fluoro-pyridine dissolved in 100 mL tetrahydrofuran (THF) at room temperature over 60 min under argon. The reaction mixture turned immediately brown. The mixture was then stirred at room temperature for 20 h. The reaction mixture was poured into cold water and extracted with ethyl acetate (×3). The combined extracts were washed with brine, dried (MgSO₄) and evaporated under reduced pressure to give a brown oil. Purification by flash chromatography (SiO₂, 10 to 70% ethyl acetate/cyclohexane) furnished (1S,5R)-3-(5-bromo-pyridin-3-yl)-3-cyano-8-aza-bicyclo[3.2.1]octane-8-carboxylic acid tert-butyl ester as a white solid.

¹H NMR (CDCl₃, TMS) δ/ppm: 1.50 (s, 9H), 2.10-2.21 (m, 2H), 2.22-2.35 (br m, 3H), 2.35-2.45 (br m, 3H), 4.30-4.52 (br m, 2H), 7.90 (t, 1H), 8.65 (2 d, 2H).

### Example C2

### Preparation of (1S,5R)-3-(5-bromo-3-pyridyl)-8-azabicyclo[3.2.1]octane-3-carbonitrile (Compound 2.074)

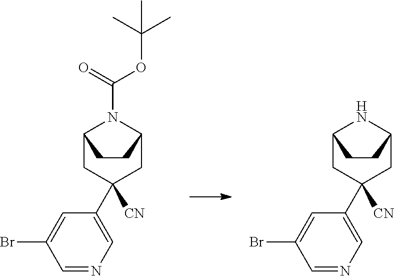 [SMILES: CC(C)(C)OC(=O)N1[C@@H]2CC[C@H]1C[C@](C#N)(c1cncc(Br)c1)C2 ; N#C[C@@]1(c2cncc(Br)c2)C[C@H]2CC[C@@H](C1)N2]

8.81 g (76.5 mmol, 5.92 mL) CF₃CO₂H (TFA) was slowly added to a solution of 3.0 g (7.65 mmol) (1S,3S,5R)-3-(5-bromo-pyridin-3-yl)-3-cyano-8-aza-bicyclo[3.2.1]octane-8-carboxylic acid tert-butyl ester in 38 mL methylene chloride at 20° C. The resultant solution was then stirred for 8 h after which the reaction was complete. The reaction mixture was concentrated and the residue treated with 20 mL diethyl ether. The precipitated TFA-salt was filtered and dried under vacuum at 40° C. for several hours to give (1S,5R)-3-(5-bromo-3-pyridyl)-8-azabicyclo[3.2.1]octane-3-carbonitrile bis-TFA salt as an off-white powder (m.p. 176-178° C.).

¹H NMR (CDCl₃, TMS) δ/ppm: 1.81-2.06 (m, 4H) 2.16-2.26 (m, 2H) 2.28-2.37 (m, 2H) 2.42-2.53 (m, 2H) 3.77 (dd, 2H) 8.00 (t, 1H) 8.63 (d, 1H) 8.73 (d, 1H).

Dissolution in water and basification to pH 9 with solid K₂CO₃ followed by thorough (×5) extraction with 200 mL dichloromethane gave a solution of the free base in dichloromethane. After combining all organic layers they were washed with saturated NaHCO₃ and brine, dried (Na₂SO₄), filtered and concentrated to give (1S,5R)-3-(5-bromo-3-pyridyl)-8-azabicyclo[3.2.1]octane-3-carbonitrile as an off-white solid (m.p. 128-130° C.).

### Example C3

### Preparation of (1S,5R)-3-(5-bromo-3-pyridyl)-8-(1,1-dimethylprop-2-ynyl)-8-azabicyclo[3.2.1]octane-3-carbonitrile (Compound 2.024)

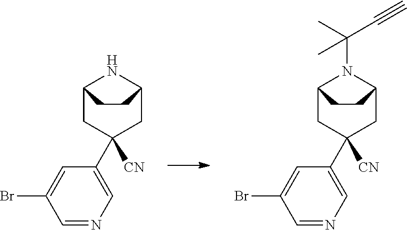 [SMILES: N#C[C@@]1(c2cncc(Br)c2)C[C@H]2CC[C@@H](C1)N2 ; C#CC(C)(C)N1[C@@H]2CC[C@H]1C[C@](C#N)(c1cncc(Br)c1)C2]

0.45 g (0.87 mmol) (1S,5R)-3-(5-bromo-3-pyridyl)-8-azabicyclo[3.2.1]octane-3-carbonitrile (bis TFA-salt) was dissolved in 5 mL DMF at 20° C. followed by addition of 0.29 g (2.08 mmol) K₂CO₃ and 13 mg (0.09) mmol sodium iodide. The resultant suspension was stirred for 20 min. Then 0.10 mL (0.95 mmol) 3-chloro-3-methyl-but-1-yne were added dropwise under stirring. The reaction mixture was heated up to 90° C. and stirred overnight. After completion the reaction mixture was filtered through Celite®, poured into water and extracted with EtOAc (3×). The organic layer was separated, washed with brine, dried over MgSO4, and concentrated. Finally, the crude material was purified by flash column chromatography to give (1S,5R)-3-(5-bromo-3-pyridyl)-8-(1,1-dimethylprop-2-ynyl)-8-azabicyclo[3.2.1]octane-3-carbonitrile as a powder (m.p. 176-178° C.).

¹H NMR (CDCl₃, TMS) δ/ppm: 1.42 (br s; 6H), 2.17 (s, 1H), 2.17-2.50 (br m, 8H), 3.90 (br s, 2H), 7.95 (br s, 1H), 8.60 (br s, 1H), 8.69 (br s, 1H).

### Example C4

### Preparation of (1S,5R)-3-(5-bromo-3-pyridyl)-8-(2-methylallyl)-8-azabicyclo[3.2.1]oct-6-ene-3-carbonitrile (Compound 5.070)

### Step 1: Preparation of (1S,5R)-3-cyano-8-aza-bicyclo[3.2.1]oct-6-ene-8-carboxylic acid tert-butyl ester

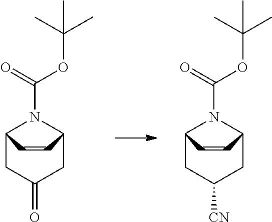 [SMILES: CC(C)(C)OC(=O)N1[C@@H]2C=C[C@H]1CC(=O)C2 ; CC(C)(C)OC(=O)N1[C@@H]2C=C[C@H]1C[C@H](C#N)C2]

1.12 g (9.52 mmol) potassium t-butoxide were suspended at 0° C. in 3 mL 1,2-dimethoxyethane (DME) under an argon atmosphere. Subsequently, a solution of 1.11 g (5.71 mmol) tosylmethyl isocyanide in 3 mL DME was added dropwise within 30 min while keeping the temperature below 5° C. The reaction mixture turned immediately brown and was stirred for an additional 1 h at 0° C. After dropwise addition of 0.58 mL (7.61 mmol) isopropanol at 0° C., the reaction mixture was stirred for an additional 30 min, before 0.85 g (3.81 mmol) (1S,5R)-3-oxo-8-azabicyclo[3.2.1]oct-6-ene-8-carboxylic acid tert-butyl ester in 2 mL DME (prepared according to Hodgson et al., Org. Lett. 2010, 12, 2834) was added dropwise within 30 min while keeping the reaction temperature below 5° C. After completion of the addition, the reaction mixture was stirred an additional 1 h at 0° C. and then allowed to warm to room temperature overnight. The reaction mixture was filtered through HyFlow (in order to remove potassium p-toluenesulfinate) and the residue was repeatedly washed with ethyl acetate. The organic layers were combined and concentrated under reduced pressure to give the crude product. The crude material was dissolved in ethyl acetate and the resultant organic solution washed with water and brine, dried (MgSO₄), filtered and concentrated. The residue was purified by flash chromatography (silica gel, 1-28% ethyl acetate/cyclohexane) to yield (1S,5R)-3-cyano-8-aza-bicyclo[3.2.1]oct-6-ene-8-carboxylic acid tert-butyl ester as light orange oil.

¹H NMR (CDCl₃, TMS) δ/ppm: 1.48 (s, 9H), 1.70-1.80 (br m, 2H), 1.80-1.97 (br m, 1H), 1.97-2.10 (br m, 1H), 2.90-3.05 (m, 1H), 4.50-4.67 (br s, 2H), 6.05-6.15 (br m, 2H).

Another ¹H NMR-signal could be detected for a second rotamer: 6.28-6.35 (br m, 2H).

### Step 2: Preparation of (1S,5R)-3-(5-bromo-pyridin-3-yl)-3-cyano-8-aza-bicyclo[3.2.1]oct-6-ene-8-carboxylic acid tert-butyl ester (Compound 5.070)

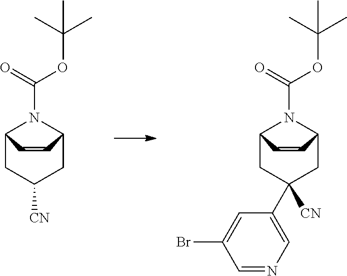 [SMILES: CC(C)(C)OC(=O)N1[C@@H]2C=C[C@H]1C[C@H](C#N)C2 ; CC(C)(C)OC(=O)N1[C@@H]2C=C[C@H]1C[C@](C#N)(c1cncc(Br)c1)C2]

Within 20 min, 35.2 mL lithium bis(trimethylsilyl)amide (1 M solution in THF) was added dropwise to a stirred solution of 7.50 g (32.0 mmol) (1S,5R)-3-cyano-8-aza-bicyclo[3.2.1]oct-6-ene-8-carboxylic acid tert-butyl ester and 5.91 g (33.6 mmol) 3-bromo-5-fluoro-pyridine in 80 mL tetrahydrofuran (THF) at −30° C. under argon atmosphere. The reaction mixture turned immediately brown. The mixture was then stirred at −30° C. for 30 min. The cooling bath was removed and the reaction mixture was allowed to warm to room temperature. The reaction mixture was stirred for an additional 2 h and then poured into cold water and extracted with ethyl acetate. The combined extracts were washed with brine, dried (MgSO₄) and evaporated under reduced pressure to give a light brown oil. Flash chromatography (silica gel, ethyl acetate/cyclohexane) of the crude product gave (1S,5R)-3-(5-bromo-pyridin-3-yl)-3-cyano-8-aza-bicyclo[3.2.1]oct-6-ene-8-carboxylic acid tert-butyl ester as light yellow oil.

¹H NMR (CDCl₃, TMS) δ/ppm: 1.55 (s, 9H), 2.12-2.25 (br m, 3H), 2.35-2.47 (br m, 1H), 4.67 (br s, 1H), 4.80 (br s, 1H), 4.80 (br s, 1H), 6.35-6.48 (br m, 2H), 7.90 (t, 1H), 8.65 (dd, 2H).

### Example C5

### Preparation of (1S,5R)-3-(5-bromo-3-pyridyl)-8-azabicyclo[3.2.1]oct-6-ene-3-carbonitrile (Compound 5.074)

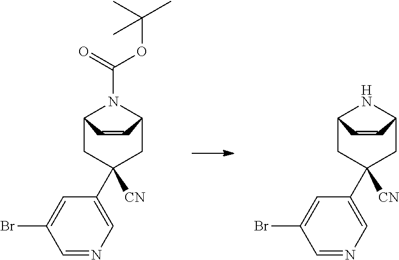 [SMILES: CC(C)(C)OC(=O)N1[C@@H]2C=C[C@H]1C[C@](C#N)(c1cncc(Br)c1)C2 ; N#C[C@@]1(c2cncc(Br)c2)C[C@H]2C=C[C@@H](C1)N2]

8.50 g (17.0 mmol) (1S,5R)-3-(5-bromo-pyridin-3-yl)-3-cyano-8-aza-bicyclo[3.2.1]oct-6-ene-8-carboxylic acid tert-butyl ester was dissolved in 87 mL dichlormethane at 20° C. Then 20.0 g (170 mmol, 13.0 mL) TFA was added slowly. The reaction mixture was stirred overnight. After completion of the reaction ethyl acetate was added. The mixture was washed with aqueous NaHCO₃ (2×) and Na₂CO₃ (2×) solution. The organic layer was separated, dried over Na₂SO₄, filtered and concentrated. The crude material was triturated with diethyl ether to give (1S,5R)-3-(5-bromo-3-pyridyl)-8-azabicyclo[3.2.1]oct-6-ene-3-carbonitrile (TFA-salt).

¹H NMR (400 MHz, DMSO-d₆) δ/ppm: 2.05-2.14 (m, 2H) 2.15-2.23 (m, 2H) 2.81 (br s, 1H) 3.32 (s, 1H) 3.93 (br s, 2H) 6.28-6.39 (m, 2H) 8.20 (t, 1H) 8.70 (d, 1H) 8.76 (d, 1H).

Dissolution in water and basification to pH 9 with solid K₂CO₃ followed by thorough (×5) extraction with 200 mL dichloromethane gave a solution of the free base in dichloromethane. After combining all organic layers they were washed with saturated NaHCO₃ and brine, dried (Na₂SO₄), filtered and concentrated to give (1S,5R)-3-(5-bromo-3-pyridyl)-8-azabicyclo[3.2.1]oct-6-ene-3-carbonitrile as an off-white solid (m.p. 152-153° C.).

### Example C6

### Preparation of (1S,5R)-3-(5-bromo-3-pyridyl)-8-(2-methylallyl)-8-azabicyclo[3.2.1]oct-6-ene-3-carbonitrile (Compound 5.052)

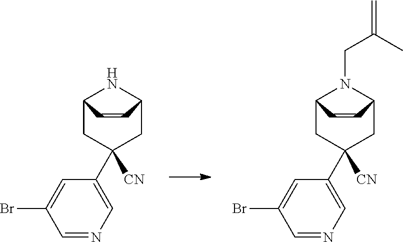 [SMILES: N#C[C@@]1(c2cncc(Br)c2)C[C@H]2C=C[C@@H](C1)N2 ; C=C(C)CN1[C@@H]2C=C[C@H]1C[C@](C#N)(c1cncc(Br)c1)C2]

0.36 g (0.90 mmol) (1S,5R)-3-(5-bromo-3-pyridyl)-8-azabicyclo[3.2.1]oct-6-ene-3-carbonitrile (TFA-salt) was suspended in 2.25 mL DMF at 20° C. followed by addition of 0.42 g (3.14 mmol) i-Pr₂NEt. The resulting solution was treated drop wise with 0.097 mL (0.09 mmol) 3-chloro-2-methyl-prop-1-ene under stirring. The reaction mixture was stirred overnight. After completion the reaction mixture was diluted with EtOAc and washed with saturated NaHCO₃ solution. The organic layer was separated. The aqueous layer was extracted thoroughly with EtOAc (2×). All organic layers were combined, dried over Na₂SO₄, filtered and concentrated. The crude material thus obtained was purified by flash chromatography to give (1S,5R)-3-(5-bromo-3-pyridyl)-8-(2-methylallyl)-8-azabicyclo[3.2.1]oct-6-ene-3-carbonitrile as a colorless oil.

¹H NMR (CDCl₃, TMS) δ/ppm: 1.79 (s, 3H) 2.17-2.35 (m, 4H) 2.86-3.00 (m, 2H) 3.73 (t, 2H) 4.87 (s, 2H) 6.18-6.35 (m, 2H) 8.00 (t, 1H) 8.62 (d, 1H) 8.74 (d, 1H).

### Example C7

### (1S,5R)-3-(5-bromo-3-pyridyl)-8-(1-methylprop-2-ynyl)-8-azabicyclo[3.2.1]oct-6-ene-3-carbonitrile (Compound 5.017)

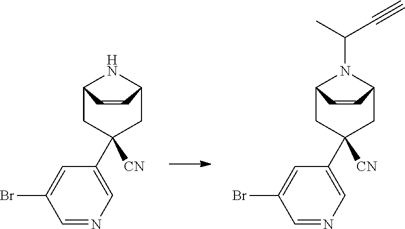 [SMILES: N#C[C@@]1(c2cncc(Br)c2)C[C@H]2C=C[C@@H](C1)N2 ; C#CC(C)N1[C@@H]2C=C[C@H]1C[C@](C#N)(c1cncc(Br)c1)C2]

0.30 g (0.58 mmol) (1S,5R)-3-(5-bromo-3-pyridyl)-8-azabicyclo[3.2.1]oct-6-ene-3-carbonitrile (TFA-salt) was suspended in 4.1 mL DMF at 20° C. under argon followed by addition of 0.32 g (2.32 mmol) K₂CO₃ and 8.7 mg (0.06 mmol) sodium iodide. The resulting suspension was stirred for 30 min. Then the suspension was treated dropwise with 0.09 g (0.64 mmol) 3-bromobut-1-yne. The reaction mixture was stirred for 20 h at 50° C. An additional amount of 3-bromobut-1-yne was added (0.09 g, 0.64 mmol) and the reaction mixture stirred for further 20 h. The reaction mixture was diluted with EtOAc and filtered with Celite®. The filtrate was concentrated and the crude material purified by flash column chromatography to give (1S,5R)-3-(5-bromo-3-pyridyl)-8-(1-methylprop-2-ynyl)-8-azabicyclo[3.2.1]oct-6-ene-3-carbonitrile as off-white powder (m.p. 114-115° C.).

¹H NMR (CDCl₃, TMS) δ/ppm: 1.38 (d, 3H) 2.25-2.37 (m, 5H) 3.29 (qd, 1H) 3.95 (q, 1H) 4.41 (q, 1H) 6.26 (dd, 1H) 6.33 (dd, 1H) 8.02 (t, 1H) 8.65 (d, 1H) 8.77 (d, 1H).

### Example C8

### (1S,5R)-3-(5-chloro-3-pyridyl)-8-(2-methylsulfanylethyl)-8-azabicyclo[3.2.1]octane-3-carbonitrile (Compound 1.044)

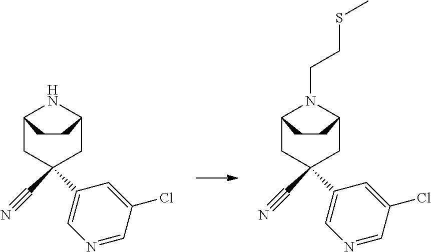 [SMILES: N#C[C@@]1(c2cncc(Cl)c2)C[C@H]2CC[C@@H](C1)N2 ; CSCCN1[C@@H]2CC[C@H]1C[C@](C#N)(c1cncc(Cl)c1)C2]

A round bottom flask was charged with 30 ml DMF at 20° C. Then 5.0 g (20.0 mmol) (1S,5R)-3-(5-chloro-3-pyridyl)-8-azabicyclo[3.2.1]octane-3-carbonitrile(prepared according to WO96/37494) and 3.96 ml (40.0 mmol) 2-chloroethyl methylsulfide was added followed by addition of 3.06 g (22.0 mmol) K₂CO₃. The resulting suspension was stirred for 30 min at 20° C. and, then the reaction mixture was heated to 80° C. and stirred for additional 5 h. After completion of the reaction, the reaction mixture was poured into water and thoroughly extracted with EtOAc (×3). The organic layer was separated, dried over MgSO₄ and concentrated. The crude product was purified by flash column chromatography to give (1S,5R)-3-(5-chloro-3-pyridyl)-8-(2-methylsulfanylethyl)-8-azabicyclo[3.2.1]octane-3-carbonitrile as orange oil.

¹H NMR (CDCl₃, TMS) δ/ppm: 2.05 (m, 2H) 2.15 (s, 3H) 2.22-2.30 (m, 4H) 2.30-2.40 (m, 2H) 2.55-2.67 (m, 4H) 3.40-3.48 (br s, 2H) 7.82 (t, 1H) 8.55 (d, 1H) 8.68 (d, 1H).

### Example C9

### Preparation of (1S,5R)-3-(5-iodo-3-pyridyl)-8-(oxetan-3-yl)-8-azabicyclo[3.2.1]oct-6-ene-3-carbonitrile (Compound 8.004)

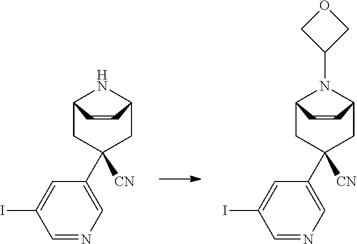 [SMILES: N#C[C@@]1(c2cncc(I)c2)C[C@H]2C=C[C@@H](C1)N2 ; N#C[C@@]1(c2cncc(I)c2)C[C@H]2C=C[C@@H](C1)N2C1COC1]

To a stirred suspension of (1S,5R)-3-(5-iodo-pyridin-3-yl)-8-aza-bicyclo[3.2.1]oct-6-ene-3-carbonitrile TFA-salt (1.00 g, 2.22 mmol) [prepared analogously to compound 5.074 starting from 3-fluoro-5-iodopyridine] in 1,2-dichloroethane (10.6 mL) at 0° C. was sequentially added 3-oxetanone (0.60 mL, 670 mg, 8.9 mmol), sodium triacetoxyborohydride (1.49 g, 6.66 mmol) and acetic acid (12 μL). After stirring of this mixture at room temperature for 20 h, the solvent was removed under reduced pressure. The residual was taken up in ethyl acetate, washed sequentially with sat. NaHCO₃ and brine, dried (Na₂SO₄) and concentrated in vacuo. Purification by flash chromatography (SiO₂, ethyl acetate/heptane) furnished the title compound as a pale-yellow solid (m.p. 170-171° C.).

### Example C10

### Preparation of (1S,5R)-8-cyclobutyl-3-(5-fluoro-3-pyridyl)-8-azabicyclo[3.2.1]oct-6-ene-3-carbonitrile (Compound 6.320)

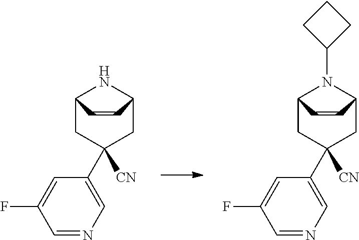 [SMILES: N#C[C@@]1(c2cncc(F)c2)C[C@H]2C=C[C@@H](C1)N2 ; N#C[C@@]1(c2cncc(F)c2)C[C@H]2C=C[C@@H](C1)N2C1CCC1]

To a stirred solution of (1S,5R)-3-(5-fluoro-pyridin-3-yl)-8-aza-bicyclo[3.2.1]oct-6-ene-3-carbonitrile TFA-salt (550 mg, 1.20 mmol) [prepared analogously to compound 5.074 starting from 3,5-difluoropyridine] in MeOH (8 mL) at 0° C. was sequentially added cyclobutanone (169 mg, 2.40 mmol), and sodium cyanoborohydride (263 mg, 3.97 mmol). After stirring of this mixture at room temperature for 20 h, the solvent was removed under reduced pressure. The residual was taken up in ethyl acetate, washed sequentially with sat. NaHCO₃ and brine, dried (Na₂SO₄) and concentrated in vacuo. Purification by flash chromatography (SiO₂, MeOH/dichloromethane) furnished the title compound as a solid (m.p. 91-93° C.).

The compounds in the following tables can be prepared analogously. The examples which follow are intended to illustrate the invention and show preferred compounds of formula (I).

TABLE A
Physical data of compounds of formula (I)

| | | LCMS | | | ¹H NMR |
|---|---|---|---|---|---|
| Compound | m.p. | ret. time | LCMS | LCMS | [CDCl₃] |
| No. | (° C.) | (min) | [M + H]⁺ | method | (δ/ppm) |
| 1.001 | 249-250 | 0.98 | 306 | B | |
| 1.002 | 220-221 | | | | |
| 1.003 | 112-113 | | | | |
| 1.004 | 140-155 | | | | |
| 1.005 | 120-160 | | | | |
| 1.006 | 205-215 | | | | |
| 1.007 | 120-125 | | | | |
| 1.008 | | 1.05 | 368 | B | |
| 1.009 | | 0.8 | 394 | H | |
| 1.010 | | 1.58 | 436 | H | |
| 1.011 | | 0.91 | 362 | H | |
| 1.012 | | 0.88 | 362 | H | |
| 1.013 | | 1.1 | 376 | H | |
| 1.014 | | 0.62 | 364 | H | |
| 1.017 | 116-117 | | | | |
| 1.019 | 92-93 | | | | |
| 1.020 | 126-129 | | | | |
| 1.022 | | 1.52 | 346 | B | |
| 1.023 | | | | | |
| 1.024 | 107-110 | | | | |
| 1.031 | | 0.81 | 320 | B | |
| 1.032 | 125-131 | 1.81 | 370 | B | |
| 1.033 | 231-232 | | | | |
| 1.034 | 127-131 | 1.21 | 332 | B | |
| 1.035 | 63-64 | 0.21 | 403 | B | |
| 1.036 | 185-189 | 1.36 | 315 | B | |
| 1.037 | 169-172 | 1.49 | 376 | B | |
| 1.038 | 217-220 | 1.78 | 402 | B | |
| 1.039 | | 1.24 | 362 | B | |
| 1.040 | 66-68 | 1.06 | 334 | B | |
| 1.041 | 72-76 | 0.99 | 314 | B | |
| 1.042 | 110-112 | | | | |
| 1.043 | 109-111 | | | | |
| 1.044 | | 0.2 | 322 | B | |
| 1.045 | ^{[1]} | | | | |
| 1.046 | 54-55 | | | | |
| 1.049 | 105-106 | | | | |
| 1.052 | 82-83 | | | | |
| 1.055 | | 0.24 | 338 | B | |
| 1.056 | ^{[1]} | | | | |
| 1.057 | | 1.38 | 343 | B | |
| 1.058 | | 1.43 | 343 | B | |
| 1.059 | | 1.82 | 341 | B | |
| 1.060 | | 0.57 | 329 | B | |
| 1.061 | | 1.29 | 327 | B | |
| 1.062 | 46-48 | | | | |
| 1.066 | | 0.39 | 353 | E | |
| 1.067 | | 0.5 | 361 | E | |
| 1.068 | 120-122 | | | | |
| 1.069 | ^{[1]} | | | | |
| 1.070 | 132-136 | | | | |
| 1.071 | 92-116 | | | | |
| 1.072 | 116-118 | | | | |
| 1.073 | 141-142 | | | | |
| 1.074 | 85-87 | | | | |
| 1.075 | ^{[1]} | | | | |
| 1.076 | | 1.03 | 332 | B | |
| 1.077 | 138-142 | 1.64 | 294 | B | |
| 1.078 | 150-153 | 1.85 | 356 | B | |
| 1.081 | 162-165 | | | | |
| 1.082 | 78-80 | | | | |
| 1.086 | 180-181 | | | | |
| 1.094 | 95-99 | | | | |
| 1.095 | 193-195 | | | | |
| 1.096 | 172-175 | | | | |
| 1.097 | 92-94 | | | | |
| 1.098 | 89-91 | | | | |
| 1.104 | 127-129 | | | | |
| 1.105 | 95-97 | | | | |
| 1.106 | 112-114 | | | | |
| 1.107 | 149-150 | | | | |
| 1.109 | 110-113 | | | | |
| 1.110 | 118-121 | | | | |
| 1.119 | 68-70 | | | | |
| 1.120 | 116-117 | | | | |
| 1.121 | 116-117 | | | | |
| 1.122 | 174-176 | | | | |
| 1.124 | 132-133 | | | | |
| 1.126 | 165-167 | | | | |
| 1.127 | 216-218 | | | | |
| 1.128 | 143-145 | | | | |
| 1.129 | 122-123 | | | | |
| 1.132 | 94-95 | | | | |
| 1.141 | 106-107 | | | | |
| 1.142 | 119-121 | | | | |
| 1.145 | 111-112 | | | | |
| 1.146 | 137-138 | | | | |
| 1.147 | 51-53 | | | | |
| 1.149 | 57-58 | | | | |
| 1.150 | 144-145 | | | | |
| 1.155 | ^{[1]} | | | | |
| 1.156 | ^{[1]} | | | | |
| 1.157 | 200-204 | | | | |
| 1.158 | 141-142 | | | | |
| 1.159 | 143-144 | | | | |
| 1.160 | 105-107 | | | | |
| 1.173 | ^{[1]} | | | | |
| 1.174 | 125-127 | | | | |
| 1.175 | 102-103 | | | | |
| 1.177 | 123-125 | | | | |
| 1.178 | 108-110 | | | | |
| 1.180 | 131-132 | | | | |
| 1.183 | 149-150 | | | | |
| 1.191 | 201-204 | | | | |
| 1.192 | 81-83 | | | | |
| 1.193 | 224 | | | | |
| 1.194 | 83.5-86.5 | | | | |
| 1.195 | 62-65 | | | | |
| 1.196 | 242-245 | | | | |
| 1.197 | 192-194 | | | | |
| 1.204 | ^{[1]} | | | | |
| 1.205 | 194-196 | | | | |
| 1.207 | 111-114 | | | | |
| 1.208 | 124-126 | | | | |
| 1.210 | 124-126 | | | | |
| 1.211 | 69-70 | | | | |
| 1.213 | 112-114 | | | | |
| 1.214 | 126-132 | | | | |
| 1.220 | 168-170 | | | | |
| 1.221 | 102-104 | | | | |
| 1.222 | 117-123 | | | | |
| 1.223 | 167-168 | | | | |
| 1.224 | 48-50 | | | | |
| 1.225 | 122-125 | | | | |
| 1.226 | 141-142 | | | | |
| 1.227 | 66-68 | | | | |
| 1.228 | 153-155 | | | | |
| 1.229 | ^{[2]} | | | | |
| 1.230 | 123 | | | | |
| 1.231 | 210-211 | | | | |
| 1.232 | 127-129 | | | | |
| 1.253 | 201-203 | | | | |
| 2.003 | ^{[3]} | | | | |
| 2.004 | 155-157 | | | | |
| 2.007 | 138-140 | | | | |
| 2.014 | | 0.71 | 408 | H | |
| 2.015 | 82-84 | | | | |
| 2.016 | 92-94 | | | | |
| 2.017 | 100-102 | | | | |
| 2.018 | 74-76 | | | | |
| 2.019 | 94-96 | | | | |
| 2.021 | ^{[3]} | | | | |
| 2.023 | | 0.77 | 392 | H | |
| 2.024 | 117-119 | | | | |
| 2.027 | | 1.05 | 376 | A | |
| 2.028 | 48-50 | | | | |
| 2.030 | 104-106 | | | | |
| 2.041 | 89-91 | | | | |
| 2.042 | 116-118 | | | | |
| 2.043 | 117-119 | | | | |
| 2.044 | 82-84 | | | | |
| 2.024 | 176-178 | | | | |
| 2.046 | | 0.69 | 405 | G | |
| 2.047 | 118-119 | 0.66 | 410 | G | |
| 2.048 | | 0.68 | 403 | G | |
| 2.049 | 104-105 | 0.57 | 380 | G | |
| 2.050 | | 0.71 | 402 | H | |
| 2.051 | 68-69 | 0.65 | 368 | H | |
| 2.052 | 52-53 | 0.62 | 347 | H | |
| 2.053 | 44-45 | 0.48 | 380 | G | |
| 2.054 | 120-121 | 1.22 | 440 | H | |
| 2.063 | | | | | 2.08-2.18 (m, 2H), |
| | | | | | 2.30-2.35 (m, 4H), 2.36-2.46 (m, |
| | | | | | 2H), 3.03-3.11 (m, 2H), |
| | | | | | 3.44-3.55 (m, 2H), |
| | | | | | 4.42-4.62 (m, 1H), 4.66-4.77 (m, |
| | | | | | 1H), 7.88-8.04 (m, 1H), |
| | | | | | 8.58-8.68 (m, 1H), |
| | | | | | 8.70-8.75 (m, 1H) |
| 2.068 | | 1.41 | 388 | H | |
| 2.069 | 92-94 | | | | |
| 2.070 | 125-127 | | | | |
| 2.071 | 166-168 | | | | |
| 2.072 | 122-124 | | | | |
| 2.073 | 163-165 | | | | |
| 2.074 | 128-130 | | | | |
| 2.075 | 103-105 | | | | |
| 2.087 | 164-166 | | | | |
| 2.088 | 109-111 | | | | |
| 2.136 | 73-75 | | | | |
| 2.194 | 75-77 | | | | |
| 2.200 | 150-151 | | | | |
| 2.204 | 63-66 | | | | |
| 2.230 | 134-136 | | | | |
| 2.240 | 85-87 | | | | |
| 2.278 | 87-89 | | | | |
| 2.282 | | 1.44 | 386 | A | |
| 2.285 | | 1.15 | 346 | A | |
| 2.288 | 117-119 | | | | |
| 2.291 | | 1.12 | 364 | A | |
| 2.292 | 73-75 | | | | |
| 2.296 | | 3.37 | 394 | I | |
| 2.297 | 99-101 | | | | |
| 2.299 | | 0.64 | 376 | H | |
| 2.303 | 61-63 | | | | |
| 2.304 | | 1.11 | 378 | A | |
| 2.305 | 156-158 | | | | |
| 2.307 | 88-90 | | | | |
| 2.312 | | 1.17 | 391 | A | |
| 2.313 | 159-160 | | | | |
| 2.314 | 168-174 | | | | |
| 2.315 | | 1.13 | 407 | A | |
| 2.316 | 115-117 | | | | |
| 2.317 | 121-123 | | | | |
| 2.318 | 134-136 | | | | |
| 2.320 | 104-105 | | | | |
| 2.321 | 180-182 | | | | |
| 2.322 | | 1.26 | 392 | A | |
| 3.003 | 60-75 | | | | |
| 3.017 | 85-86 | | | | |
| 3.042 | 130-132 | | | | |
| 3.043 | 64-66 | | | | |
| 3.044 | | 0.26 | 306 | H | |
| 3.049 | | 0.36 | 318 | H | |
| 3.052 | | 0.28 | 286 | H | |
| 3.071 | 115-117 | | | | |
| 3.072 | 80-82 | | | | |
| 3.074 | | 0.26 | 232 | H | |
| 3.223 | 135-137 | | | | |
| 4.003 | 117-120 | | | | |
| 4.017 | 101-104 | | | | |
| 4.019 | 100-101 | | | | |
| 4.020 | 129-133 | | | | |
| 4.021 | ^{[3]} | | | | |
| 4.042 | 122-126 | | | | |
| 4.043 | 120-122 | | | | |
| 4.050 | | 0.57 | 356 | G | |
| 4.052 | 72-74 | | | | |
| 4.069 | ^{[3]} | | | | |
| 4.070 | | 0.99 | 346 | G | |
| 4.073 | 151-154 | | | | |
| 4.074 | 94-96 | | | | |
| 4.083 | 130-131 | | | | |
| 4.104 | 111-113 | | | | |
| 4.113 | ^{[3]} | | | | |
| 4.181 | 70-80 | | | | |
| 4.203 | ^{[3]} | | | | |
| 4.223 | 106-108 | | | | |
| 4.228 | 140-142 | | | | |
| 4.229 | ^{[3]} | | | | |
| 5.003 | 131-132 | | | | |
| 5.004 | 144-145 | | | | |
| 5.006 | 220-221 | 1.01 | 394 | H | |
| 5.009 | | 0.99 | 434 | H | |
| 5.015 | | 0.93 | 389.59 | C | |
| 5.016 | | 0.53 | 360 | H | |
| 5.017 | | 0.6 | 344 | H | |
| 5.018 | | 0.56 | 348.14 | C | |
| 5.019 | 95-103 | | | | |
| 5.020 | 135-136 | | | | |
| 5.021 | | 0.54 | 346 | H | |
| 5.023 | | 0.88 | 390.20 | C | |
| 5.024 | | 0.80 | 356.23 | C | |
| 5.025 | | 0.87 | 372.25 | C | |
| 5.026 | | 0.81 | 375.89 | C | |
| 5.027 | | 0.34 | 374 | H | |
| 5.027 | | 0.53 | 374.36 | C | |
| 5.028 | | 0.66 | 357.63 | C | |
| 5.029 | | 0.56 | 360.01 | C | |
| 5.030 | | 1.08 | 408.26 | C | |
| 5.031 | | 0.63 | 363 | H | |
| 5.041 | | 0.74 | 357 | H | |
| 5.042 | | 0.52 | 330 | G | |
| 5.043 | | 0.87 | 356 | G | |
| 5.044 | | 0.53 | 364 | H | |
| 5.045 | | 1.00 | 424 | G | |
| 5.050 | | 0.66 | 402 | H | |
| 5.052 | | 0.51 | 344 | H | |
| 5.055 | 112-113 | | | | |
| 5.060 | | 0.41 | 371 | H | |
| 5.063 | | 0.7 | 348 | H | |
| 5.064 | | 0.37 | 375 | H | |
| 5.065 | | 0.46 | 360 | H | |
| 5.068 | | 1.29 | 386 | H | |
| 5.069 | | 0.87 | 364 | G | |
| 5.070 | | 1.77 | 392 | B | |
| 5.071 | 165-167 | | | | |
| 5.072 | 110-112 | | | | |
| 5.074 | | 0.31 | 292 | G | |
| 5.075 | | 0.34 | 334 | H | |
| 5.082 | 84-86 | | | | |
| 5.104 | 93-95 | | | | |
| 5.223 | 106-108 | | | | |
| 5.238 | | 0.49 | 356 | H | |
| 5.239 | 89-91 | | | | |
| 5.240 | | 0.56 | 392 | H | |
| 5.244 | | 0.45 | 388 | H | |
| 5.245 | | 0.42 | 388 | H | |
| 5.251 | | 0.4 | 394 | G | |
| 5.278 | 98-99 | | | | |
| 5.280 | 84-89 | | | | |
| 5.285 | | 0.52 | 344 | H | |
| 5.288 | 142-143 | | | | |
| 5.292 | | 0.53 | 378 | G | |
| 5.297 | 105-107 | | | | |
| 5.298 | 109-112 | | | | |
| 5.299 | 128-131 | | | | |
| 5.301 | 66-72 | | | | |
| 5.302 | 66-72 | | | | |
| 5.306 | 85-89 | | | | |
| 5.308 | 157-159 | | | | |
| 5.309 | 119-143 | | | | |
| 5.310 | 123-127 | | | | |
| 5.311 | | 1.95 | 468 | H | |
| 5.313 | 171-176 | | | | |
| 5.314 | 188-190 | | | | |
| 5.319 | | | | | 0.67-0.75 (m, 2H), |
| | | | | | 0.82-0.92 (m, 2H), 2.15-2.31 (m, |
| | | | | | 4H), 3.25 (s, 3H), 4.18 (br |
| | | | | | s, 2H), 6.38 (s, 2H), |
| | | | | | 7.95-8.00 (m, 1H), 8.59.8.62 (m, |
| | | | | | 1H), 8.67-8.70 (m, 1H) |
| 5.320 | 94-95 | | | | |
| 5.321 | 147-149 | | | | |
| 6.003 | 106-108 | | | | |
| 6.004 | 160-162 | | | | |
| 6.017 | 92-95 | | | | |
| 6.042 | 135-137 | | | | |
| 6.043 | 96-98 | | | | |
| 6.044 | | 0.32 | 304 | H | |
| 6.049 | | 0.51 | 316 | H | |
| 6.052 | | 0.31 | 284 | H | |
| 6.063 | | 0.43 | 288 | H | |
| 6.071 | 132-133 | | | | |
| 6.072 | | 0.23 | 258 | H | |
| 6.074 | 72-74 | | | | |
| 6.082 | 65-67 | | | | |
| 6.297 | | 0.75 | 304 | H | |
| 6.300 | | 0.29 | 304 | H | |
| 6.320 | 91-93 | | | | |
| 7.043 | 117-119 | | | | |
| 7.070 | 122-124 | | | | |
| 7.223 | 144-145 | | | | |
| 8.003 | 135-136 | | | | |
| 8.004 | 170-171 | | | | |
| 8.018 | 97-98 | | | | |
| 8.043 | 142-143 | | | | |
| 8.044 | | 0.59 | 412 | G | |
| 8.063 | 115-116 | | | | |
| 8.074 | 207-208 | | | | |
| 8.082 | 99-100 | | | | |
| 8.104 | 112-113 | | | | |
| 8.297 | 154-155 | | | | |

^{[1]}Analytical data matching those described in WO96/37494.
^{[2]}Analytical data matching those described in WO97/43286
^{[3]}Analytical data matching those described in WO98/25924.

### BIOLOGICAL EXAMPLES

### Example B1

### Control of Insects Resistant to Neonicotinoids

The level of resistance and therefore the impact on the performance of the insecticide can be measured by the use of a ‘Resistance Factor’. The resistance factor can be calculated by dividing the concentration of an insecticide that provides a set level of mortality (i.e. 80%) for the ‘resistant’ strain with the concentration of the same insecticide that provides the same level of mortality for the ‘susceptible’ insect of the same species and life-stage. Although there are no set rules, a low value (less than or equal to 20) indicates no cross-resistance and only natural levels of variation and a high value (greater than or equal to 64) provides strong evidence of cross-resistance.

In order to obtain neonicotinoid resistant insects, a researcher is to locate a host crop and geographical region where the relevant resistance had been reported in literature (e.g. Myzus persicae—peach orchards of France. Bemisia tabaci—protected vegetables in Spain). Live samples of the insect are then collected from the locations/host crops and transported back to a laboratory, where breeding colonies would be established. Non-resistant individuals with the colonies are eliminated to provide a homologous-resistant population. This is achieved by either establishing a clonal population of insects from a single resistant individual (e.g. Myzus persicae) or by repeatedly exposing the colony to a dose of insecticide which kills susceptible insects, whilst leaving resistant insects unaffected. The resistant phenotype of the insect colony is determined either by conducting a full dose response bioassay (examples of which can be found on the IRAC web-site and below) with a neonicotinoid insecticide and comparing the bioassay results to similar bioassay results for a known susceptible colony of the same species. Alternatively the resistance genotype of the individual insects can be determined by molecular techniques (e.g. PCR) if the resistance mechanism for the relevant species is known.

a) Neonicotinoid Resistant Strain of the Green Peach Aphid (Myzus persicae)
a.1) Myzus persicae Strains Utilised:
Standard screening strain of Myzus persicae (Neonicotinoid susceptible)
FRC-P strain of Myzus persicae (Neonicotinoid resistant) obtained from peach orchards in Southern France
   a2) Bioassay Methods Utilised

### a.2.1) Bioassay, Method A:

Myzus persicae: mixed population, contact activity, curative on pea seedlings

Pea seedlings were infested with an aphid population of mixed ages and treated with the test solutions in a spray chamber. 6 days after treatment samples were checked for mortality.

Application rates: 200 ppm, 50 ppm, 12.5 ppm, 3 ppm and 0.8 ppm.

### a.2.2) Dose-Response Bioassay, Method B:

Test pots (45 mm diameter) were prepared with discs of Chinese cabbage on tap water agar adapted from Herron et al (Aust J Entomol 37:70-73 (1998)). Mixed age aphids (numbering 20-30) were transferred to the dishes and allowed to settle for 24 h at 21 degrees C. with a 16:8 h light/dark regime. Dead individuals were removed prior to application. Serial dilutions of insecticide were applied using a Potter precision laboratory spray tower (Burkard Scientific, Uxbridge, UK), before sealing each pot with a lid. Each treatment replicate was sprayed with 3 mL solution at 0.6 bar with a 3 s settling time (equivalent to approximately 400 L ha-1). A minimum of five insecticide concentrations and three replicates per treatment were utilised in each test. Aphid mortality is assessed at 72 hours after treatment (depending on insecticide mode of action). LC50 values were calculated by LOGIT analysis (using ACSAPwin program).

### a.3) Results

The following compounds, according to the present invention, gave at least 80% control of the FRC-P (Neonicotinoid resistant) strain of Myzus persicae at 200 ppm and exhibited a resistance factor of ≦20: 1.003, 1.005, 1.006, 1.016, 1.020, 1.023, 1.024, 1.031, 1.034, 1.043, 1.044, 1.046, 1.050, 1.055, 1.060, 1.061, 1.065, 1.066, 1.072, 1.073, 1.075, 1.076, 1.082, 1.087, 1.090, 1.097, 1.104, 1.107, 1.108, 1.117, 1.149, 1.175, 1.177, 1.182, 1.194, 1.203, 1.204, 1.223, 1.224, 1.232, 2.003, 2.017, 2.019, 2.021, 2.047, 2.064, 2.072, 2.073, 2.087, 2.194, 3.223, 4.003, 4.020, 4.021, 4.042, 4.073, 5.003, 5.015, 5.017, 5.018, 5.026, 5.031, 5.042, 5.043, 5.050, 5.075, 5.082, 5.301, 5.302, 5.320, 7.204, and 7.223.

Thiacloprid and Imidacloprid failed to give 80% control of the FRC-P (Neonicotinoid resistant) strain of Myzus persicae at 200 ppm and both exhibited a Resistance Factor (RF₈₀) of >64.

b) Neonicotinoid and Pyrethroid Resistant Strain of the Tobacco Whitefly (Bemisia tabaci)
b.1) Bemisia tabaci Strains Utilised:
Standard screening strain of Bemisia tabaci (Neonicotinoid susceptible)
Q-biotype strain of Bemisia tabaci (Neonicotinoid resistant) originally provided by Rothamsted Research, UK.

### b.2) Bioassay Methods Utilised:

### b.2.1) Bioassay, Method A:

Bemisia tabaci: residual activity, preventive egg lay

Cotton seedlings, with all but a single leaf removed are treated with the diluted test solutions in a turn table spray chamber. 24 hours after drying, they are infested with 20 adult whitefly. 3 days after exposure, the total number of adult whitefly and the total number of whitefly eggs laid on the leaf are counted. Percentage control of egg lay is calculated and corrected for control mortality.

Application rates: 200 ppm, 50 ppm, 12.5 ppm, 3 ppm and 0.8 ppm.

### b.2.2) Dose-Response Bioassay, Method B:

Test pots (45 mm diameter) were prepared with discs of cotton leaf on tap water agar adapted from Herron et al (Aust J Entomol 37:70-73 (1998)). Serial dilutions of insecticide were applied using a Potter precision laboratory spray tower (Burkard Scientific, Uxbridge, UK). Each treatment replicate was sprayed with 3 mL solution at 0.6 bar with a 3 s settling time (equivalent to approximately 400 L ha-1). A minimum of five insecticide concentrations and three replicates per treatment were utilised in each test. After the test solutions had dried, adult whitefly (numbering 20-30) were transferred to the pots, before it was sealed with a lid and turned upside down (whitefly on underside of leaf surface) for 72 hours after treatment at 24 degrees C. with a 16:8 h light/dark regime. Whitefly mortality is evaluated and LC50 values were calculated by LOGIT analysis (using ACSAPwin program).

### b.3) Results

The following compounds, according to the present invention, gave at least 80% control of the Q-biotype (Neonicotinoid resistant) strain of Bemisia tabaci at 200 ppm and exhibited a resistance factor of ≦20: 1.003, 1.016, 1.024, 1.042, 1.060, 1.065, 1.072, 1.075, 1.108, 1.117, 1.182, 1.203, 1.204, 1.223, 1.224, 2.014, 2.017, 2.023, 2.024, 2.030, 2.043, 2.049, 2.050, 2.072, 2.073, 2.075, 2.194, 2.285, 2.291, 2.297, 2.320, 3.017, 3.042, 3.043, 3.044, 3.049, 3.223, 4.017, 4.019, 4.020, 4.042, 4.050, 4.052, 4.104, 5.017, 5.018, 5.019, 5.031, 5.044, 5.050, 5.052, 5.063, 5.071, 5.072, 5.082, 5.223, 5.280, 5.285, 5.299, 5.301, 5.302, 5.313, 5.320, 5.321, 6.017, 6.042, 6.043, 6.044, 6.052, 6.071, 6.072, 7.223, 8.003, and 8.082.

Thiacloprid and Imidacloprid failed to give 80% control of the Q-biotype (Neonicotinoid resistant) strain of Bemisia tabaci at 200 ppm and both exhibited a resistance factor of >64.

1. A method of controlling insects from the order hemiptera, which insects are resistant to a neonicotinoid insecticide, which method comprises applying to said neonicotinoid resistant insects a compound of formula (I): 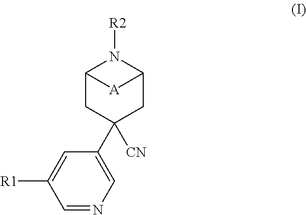
wherein
A is —CH₂—CH₂— or —CH═CH—; R¹ is halogen; and R² is hydrogen, formyl, cyano, hydroxy, NH₂, C₁-C₆alkyl (optionally substituted by aryl, aryloxy, heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl (optionally substituted by aryl or heteroaryl, which themselves can be optionally substituted by one to three substituents independently selected from halogen, cyano, nitro, C₁-C₄alkyl, C₁-C₄haloalkyl, and C₁-C₄alkoxy), C₁-C₄alkoxy(C₁-C₄)alkoxy(C₁-C₄)alkyl, C₁-C₆alkylcarbonyl(C₁-C₆)alkyl, C₁-C₄alkoxyimino(C₁-C₄)alkyl, C₁-C₄haloalkoxy(C₁-C₄)alkyl, C₁-C₆alkoxycarbonyl(C₁-C₆)alkyl, C₁-C₄alkoxy(C₁-C₄)alkoxycarbonyl(C₁-C₆)alkyl, hydroxycarbonyl(C₁-C₆)alkyl, aryloxycarbonyl(C₁-C₆)alkyl (wherein the aryl group can be optionally substituted by one or two substituents independently selected from halogen, cyano, nitro, C₁-C₄alkyl, C₁-C₄ haloalkyl, and C₁-C₄alkoxy), C₁-C₄alkylaminocarbonyl(C₁-C₆)alkyl, di(C₁-C₄alkyl)aminocarbonyl(C₁-C₆)alkyl, C₁-C₄haloalkylaminocarbonyl(C₁-C₆)alkyl, di(C₁-C₄haloalkyl)aminocarbonyl-C₁-C₆alkyl, C₁-C₂alkoxy(C₂-C₄)alkylaminocarbonyl(C₁-C₄)alkyl, C₂-C₆alkenyloxycarbonyl(C₁-C₆)alkyl, C₃-C₆alkynyloxycarbonyl(C₁-C₆)alkyl, (R³O)₂(O═)P(C₁-C₆)alkyl where R³ is hydrogen, C₁-C₄alkyl or benzyl, C₃-C₇cycloalkyl (optionally substituted by one to three substituents independently selected from C₁-C₄alkyl, C₁-C₄haloalkyl, and C₁-C₄alkoxy and, additionally, one of the ring member units can optionally represent C═O or C═NR⁴ where R⁴ is hydrogen, C₁-C₄alkyl, C₁-C₄haloalkyl, C₁-C₄cyanoalkyl, C₁-C₄alkoxy, or C₃-C₆cycloalkyl), C₃-C₇halocycloalkyl, C₃-C₇cycloalkenyl (optionally substituted by one or two substituents independently selected from C₁-C₄alkyl, and C₁-C₄haloalkyl, and, additionally, one of the ring member units can optionally represent C═O), C₃-C₇halocycloalkenyl, C₁-C₆alkyl-S(═O)n¹(C₁-C₆)alkyl where n¹ is 0, 1 or 2 (optionally substituted by aryl or heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₃-C₆alkenyl, C₃-C₆haloalkenyl, aryl(C₃-C₆)alkenyl, C₃-C₆alkynyl, C₃-C₆haloalkynyl, aryl(C₃-C₆)alkynyl, C₃-C₆hydroxyalkynyl, C₁-C₆alkoxycarbonyl (optionally substituted by one to three substituents independently selected from halogen, hydroxy, cyano, C₁-C₄alkoxy, C₁-C₄haloalkyl, and aryl), aryloxycarbonyl (optionally substituted by one to three substituents independently selected from halogen, cyano, nitro, C₁-C₄alkyl, C₁-C₄haloalkyl, C₁-C₄alkoxy), C₃-C₆alkenyloxycarbonyl, C₃-C₆alkynyloxycarbonyl, C₁-C₆alkylcarbonyl, C₁-C₆haloalkylcarbonyl, aminocarbonyl, C₁-C₆alkylaminocarbonyl, di(C₁-C₆)alkylaminocarbonyl, aminothiocarbonyl, C₁-C₆alkylaminothiocarbonyl, di(C₁-C₆)alkylaminothiocarbonyl, C₁-C₆alkoxy, C₃-C₆alkenyloxy, C₃-C₈alkynyloxy, aryloxy (optionally substituted by one to three substituents independently selected from halogen, cyano, nitro, C₁-C₄alkyl, C₁-C₄haloalkyl, and C₁-C₄alkoxy), C₁-C₆alkylamino, di(C₁-C₆)alkylamino, C₃-C₆cycloalkylamino, C₁-C₄alkylthio, C₁-C₄alkylsulfinyl, C₁-C₄alkylsulfonyl, C₁-C₄haloalkylsulfonyl, aryl-S(═O)n² (optionally substituted by one or two substituents independently selected from halogen, nitro, and C₁-C₄alkyl) where n² is 0, 1 or 2, aryl (optionally substituted by one to three substituents independently selected from halogen, cyano, nitro, C₁-C₄alkyl, C₁-C₄haloalkyl, C₁-C₄alkoxy, and C₁-C₄haloalkoxy), heteroaryl (optionally substituted by one to three substituents independently selected from halogen, cyano, nitro, C₁-C₄alkyl, C₁-C₄haloalkyl, C₁-C₄alkoxy, and C₁-C₄haloalkoxy), heterocyclyl (optionally substituted by one to three substituents independently selected from halogen, cyano, nitro, C₁-C₄alkyl, C₁-C₄haloalkyl, C₁-C₄alkoxy, and C₁-C₄haloalkoxy, and, additionally, a ring member unit can optionally represent C═O or C═NR⁵ where R⁵ is hydrogen, C₁-C₄ alkyl, C₁-C₄ haloalkyl, C₁-C₄ cyanoalkyl, C₁-C₄ alkoxy, or C₃-C₆ cycloalkyl), (C₁-C₆alkylthio)carbonyl, (C₁-C₆alkylthio)thiocarbonyl, C₁-C₆alkyl-S(═O)n³(═NR⁶)—C₁-C₄alkyl wherein R⁶ is hydrogen, cyano, nitro, C₁-C₄alkyl and n³ is 0 or 1, or R² represents a group “—C(R⁷)(R⁸)(R⁹)” wherein R⁷ is C₁-C₄alkyl, C₁-C₄haloalkyl, or cyclopropyl, R⁸ is hydrogen, C₁-C₄alkyl, C₁-C₄haloalkyl, or cyclopropyl, preferably hydrogen, R⁹ is cyano, C₁-C₄alkyl, C₂-C₆alkenyl, C₂-C₆haloalkenyl, C₁-C₄alkoxy, C₂-C₆alkynyl, C₂-C₄alkoxycarbonyl, C₁-C₄alkylaminocarbonyl, di(C₁-C₃alkyl)aminocarbonyl, C₁-C₂haloalkylaminocarbonyl, C₃-C₆alkenyloxycarbonyl, C₃-C₄alkynyloxycarbonyl, or C₁-C₃alkylcarbonyl; (arylthio)carbonyl(C₁-C₆)alkyl (wherein aryl can be optionally substituted by one to three substituents independently selected from halogen, cyano, nitro, C₁-C₄alkyl, C₁-C₄haloalkyl, and C₁-C₄alkoxy), (C₁-C₃alkylthio)carbonyl(C₁-C₆)alkyl (optionally substituted by aryl or heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), or an agrochemically acceptable salt, N-oxide or isomer thereof.

2. A method according to claim 1 wherein A is —CH═CH—.

3. A method according to claim 1 wherein R¹ is F, I, or Br.

4. A method according to claim 1 wherein R² is hydrogen, formyl, C₁-C₆alkyl (optionally substituted by phenyl, heteroaryl (wherein heteroaryl is pyridyl, thiophenyl, oxazolyl, isoxazolyl, oxadiazolyl, thiazolyl, isothiazolyl or thiadiazolyl) or heterocyclyl (wherein heterocyclyl is tetrahydrofuranyl, [1,3]dioxolanyl, oxetanyl, thietanyl, 1-oxo-thietanyl or 1,1-dioxo-thietanyl), which themselves can be optionally substituted by one or two substituents independently selected from halogen, cyano, C₁-C₄alkyl, C₁-C₄haloalkyl, and C₁-C₄alkoxy), C₁-C₆haloalkyl (optionally substituted by one or two substituents independently selected from C₁-C₄-alkoxy, tri(C₁-C₄alkyl)silyloxy), and C₃-C₅alkenyl), C₁-C₆cyanoalkyl, C₁-C₆alkoxy(C₁-C₆)alkyl, C₁-C₆alkylcarbonyl(C₁-C₆)alkyl, C₁-C₆alkoxycarbonyl(C₁-C₆)alkyl, C₁-C₄alkylaminocarbonyl(C₁-C₆)alkyl, di(C₁-C₄alkyl)aminocarbonyl(C₁-C₆)alkyl, C₁-C₄haloalkylaminocarbonyl(C₁-C₆)alkyl, C₂-C₆alkenyloxycarbonyl(C₁-C₆)alkyl, C₃-C₆alkynyloxycarbonyl(C₁-C₃)alkyl, C₃-C₆cycloalkyl (optionally substituted by one or two substituents independently selected from C₁-C₄alkyl, and C₁-C₄haloalkyl, and, additionally, one of the ring member units can optionally represent C═O), C₃-C₆halocycloalkyl, C₄-C₇cycloalkenyl (optionally substituted by one or two substituents independently selected from C₁-C₄alkyl, and C₁-C₄haloalkyl, and, additionally, one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, phenyl(C₃-C₆)alkenyl, C₃-C₆alkynyl, C₃-C₆haloalkynyl, C₁-C₆alkoxy, heterocyclyl (wherein heterocyclyl is oxetanyl, 3-tetrahydrofuranyl-2-one, 1,1-dioxo-thietanyl, 1-oxo-thietanyl or thietanyl, and is optionally substituted by one or two substituents independently selected from halogen, cyano, C₁-C₄alkyl, C₁-C₄haloalkyl, and C₁-C₄alkoxy, and, additionally, a ring member unit can optionally represent C═O or C═NR⁵ where R⁵ is C₁-C₄ alkyl, or C₁-C₄ alkoxy); or R² represents the group “—C(R⁷)(R⁸)(R⁹)” wherein R⁷ is C₁-C₄alkyl, C₁-C₄haloalkyl, or cyclopropyl; R⁸ is hydrogen, C₁-C₄alkyl, C₁-C₄haloalkyl, or cyclopropyl, preferably hydrogen; and R⁹ is cyano, C₁-C₄alkyl, C₂-C₆alkenyl, C₂-C₆haloalkenyl, C₁-C₄alkoxy, C₂-C₅alkynyl, C₂-C₄alkoxycarbonyl, C₁-C₄alkylaminocarbonyl, di(C₁-C₃alkyl)aminocarbonyl, C₁-C₂haloalkylaminocarbonyl, C₃-C₆alkenyloxycarbonyl, C₃-C₄alkynyloxycarbonyl, or C₁-C₃alkylcarbonyl.

5. A method according to claim 1 wherein undesired insects from the order Hemiptera which are resistant to one or more of the neonicotinoid insecticides are controlled but beneficial arthropods are not affected.

6. A method according to claim 1 wherein the method comprises applying a compound of formula (I) and one or more beneficial arthropods.

7. A method according to claim 5 wherein the beneficial arthropods are one or more beneficial insects or predatory mites selected from Orius insidiosus, Orius laevigatus, Orius majusculus, Coccinella septempunctata, Adalia bipunctata, Amblydromalus limonicus, Amblyseius andersoni, Amblyseius barkeri, Amblyseius califomicus, Amblyseius cucumeris, Amblyseius montdorensis, Amblyseius swirskii, Phytoseiulus persimilis, Syrphus spp., and Phytoseiulus persimilis.

8. A method according to claim 1 wherein the neonicotinoid resistant insects are from the Aleyrodidae family or the Aphididae family.

9. A compound of formula (IL) 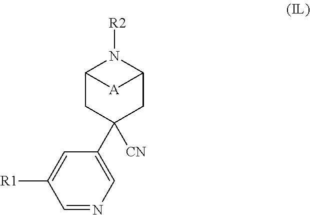
wherein A is —CH₂—CH₂— or —CH═CH—; and
R¹ is F or I, when R² is C₁-C₅haloalkyl, C₃-C₅haloalkenyl, C₃-C₅haloalkynyl, C₃-C₅alkenyl, C₃-C₅alkynyl, C₃-C₇cycloalkyl, or C₅-C₇cycloalkenyl; or
R¹ is F, Br or I, when R² is ethyl, propyl, isopropyl, 2-methylprop-2-enyl, C₁-C₂alkoxy(C₁-C₂)alkyl, C₁-C₂alkyl-S(═O)n⁵(C₁-C₂)alkyl where n⁵ is 0, 1 or 2 (preferably n⁵ is 0), C₃haloalkenyl, C₂-C₄alkoxycarbonyl(C₁-C₂)alkyl, or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is cyano, C₂alkenyl, C₂haloalkenyl, C₂alkynyl, C₂-C₄alkoxycarbonyl, or C₃-C₅alkenyloxycarbonyl; or
wherein A is —CH₂—CH₂—; and R¹ is Cl, when R² is propyl, 2-methylprop-2-enyl, C₁-C₂alkyl-S(═O)n⁵(C₁-C₂)alkyl where n⁵ is 0, 1 or 2 (preferably n⁵ is 0), or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is C₂alkenyl, C₂haloalkenyl, C₂alkynyl, or C₃-C₅alkenyloxycarbonyl, provided that R² is not 2-methylbut-3-yn-2-yl; or
wherein A is —CH═CH—; and R¹ is Cl, when R² is ethyl, propyl, isopropyl, 2-methylprop-2-enyl, C₁-C₂alkoxy(C₁-C₂)alkyl, C₁-C₂alkyl-S(═O)n⁵(C₁-C₂)alkyl where n⁵ is 0, 1 or 2 (preferably n⁵ is 0), C₃haloalkenyl, C₂-C₄alkoxycarbonyl(C₁-C₂)alkyl, or R² represents the group “—C(R⁷)(R⁸)(R⁹)” wherein R⁷ is C₁-C₂alkyl; R⁸ is hydrogen or C₁-C₂alkyl, preferably hydrogen; and R⁹ is C₂alkenyl, C₂haloalkenyl, C₂alkynyl, C₂-C₄alkoxycarbonyl, or C₃-C₅alkenyloxycarbonyl; or
wherein A is —CH₂—CH₂— or —CH═CH—; and R¹ is F, Cl, Br or I; and R² represents cyclobutyl, cyclopentenyl, cyclohexenyl, cycloheptenyl, 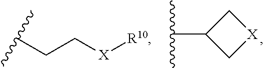
—CH₂C(R¹¹)═CH₂ or —CH₂CH═CH(R¹¹), where X is O, S, S(O) or S(O)₂, R¹⁰ is C₁-C₄alkyl, and R¹¹ is halogen or methyl;
or wherein A=—CH₂—CH₂— and R¹ and R² are as defined below:

| R¹ | R² |
|---|---|
| Cl | isoxazol-4-ylmethyl |
| Cl | 3-oxetanyl |
| Cl | 3-thietanyl |
| Br | 1-methylprop-2-ynyl |
| Br | 1,1-dimethylprop-2-ynyl |
| Br | 2-bromoallyl |
| Br | 2,2-difluoroethyl |
| Br | 4,4,4-trifluorobutyl |
| Br | ethyl |
| Br | isopropyl |
| Br | 1-methylallyl |
| Br | 2-chloroallyl |
| Br | cyclobutyl |
| F | propargyl |
| F | 2,2-difluoroethyl |
| F | 2-methylsulfanylethyl |
| F | 2-methoxy-1-methyl-2-oxo-ethyl |
| I | 2,2-difluoroethyl |

or wherein A=—CH═CH— and R¹ and R² are as defined below:

| R¹ | R² |
|---|---|
| Cl | 1-methylprop-2-ynyl |
| Cl | but-3-ynyl |
| Cl | 4,4,4-trifluorobutyl |
| Cl | 2-methylallyl |
| Cl | 2-fluoroethyl |
| Br | 1-methylprop-2-ynyl |
| Br | 2-methoxyethyl |
| Br | but-3-ynyl |
| Br | 2-methylsulfanylethyl |
| Br | 4,4,4-trifluorobutyl |
| Br | 2-methylallyl |
| Br | 2-fluoroallyl |
| Br | cyclopropyl |
| Br | ethyl |
| Br | (Z)-3-chlorobut-2-enyl |
| Br | 1-methylallyl |
| Br | tetrahydrofuran-2-ylmethyl |
| Br | (S)-1-methylprop-2-ynyl |
| Br | (R)-1-methylprop-2-ynyl |
| Br | cyclobutyl |
| Br | tetrahydropyran-4-yl |
| F | oxetan-3-yl |
| F | 1-methylprop-2-ynyl |
| F | propargyl |
| F | 2,2-difluoroethyl |
| F | 2-methylsulfanylethyl |
| F | 2-methylallyl |
| F | ethyl |
| F | allyl |
| F | cyclobutyl |
| I | 2,2,2-trifluoroethyl |
| I | oxetan-3-yl |
| I | 2-methoxyethyl |
| I | 2,2-difluoroethyl |
| I | 2-methylsulfanylethyl |
| I | 2-fluoroallyl |
| I | allyl |
| I | 2-fluoroethyl |
| I | 2-chloroallyl |

or an agrochemically acceptable salt, N-oxide or isomer thereof.

10. A compound of the formula 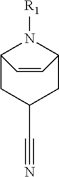
wherein R¹ is as defined claim 9; or a compound of the formula 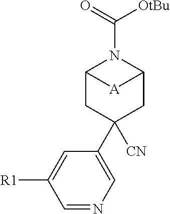
wherein A is —CH₂—CH₂— or —CH═CH—; and R¹ is F, Cl, Br or I; or a compound of the formula 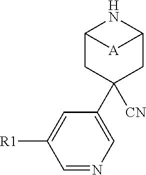
wherein A is —CH₂—CH₂— or —CH═CH—; and R¹ is F or I.

11. An insecticidal composition comprising an insecticidally effective amount of a compound of formula (IL) as defined in claim 9.
